(19) United States
(12) Patent Application Publication
(10) Pub. No.: US 2003/0171279 A1
(43) Pub. Date: Sep. 11, 2003

(54) Methods and composition concerning herpesvirus Us3 and BAD-involved apoptosis

(76) Inventors: Joshua Munger, Chicago, IL (US); Bernard Roizman, Chicago, IL (US)

Correspondence Address: Charles P. Landrum, FULBRIGHT & JAWORSKI L.L.P., SUITE 2400, 600 CONGRESS AVENUE, AUSTIN, TX, 78701-3271, US

(21) Appl. No.: 10/209,967

(22) Filed: Jul. 31, 2002

Related U.S. Application Data
(60) Provisional application No. 60/308,929, filed on Jul. 31, 2001.

(51) Int. Cl. A61K 38/16; A61K 38/10; A61K 38/08; A61K 38/06

(52) U.S. Cl. 514/12; 514/13; 514/14; 514/15; 514/16; 514/17; 514/18

(57) ABSTRACT

The present invention concerns methods of compositions for inhibiting or inducing apoptosis in a cell. The methods and compositions concern either the herpesviral protein U_{S}3, the cellular pro-apoptotic polypeptide BAD, or modulators thereof to modulate apoptosis in a cell.

This application claims the priority of U.S. Provisional Application Ser. No. 60/308,929, filed Jul. 31, 2001, the entire disclosure of which is specifically incorporated herein by reference.

The government may own rights in this application by virtue of federal funding under grant numbers AI124009 from the National Institute of Allergy and Infectious Disease and CA47451, CA87761, CA83939, CA71933, and CA78766 from the National Cancer Institute.

### BACKGROUND OF THE INVENTION

I. Field of the Invention

The present invention relates to the fields of virology and cell biology generally, and more specifically, it addresses mechanisms for growth control in eukaryotic cells. In particular, there are provided methods and compositions involving the inhibition of the pro-apoptotic protein BAD by U_{S}3.

II. Related Art

The control of host cell gene expression, and often the control of genes involved in DNA replication, are integral parts of the life cycle of a virus. However, recent evidence suggests that most eukaryotic cells respond to viral disruption of normal cellular physiology by undergoing programmed cell death, also known as “apoptosis” (White, 1993). To counteract apoptosis, many viruses have evolved mechanisms to block host cell death (Clem and Miller, 1994; White and Gooding, 1994). In several cases, viral genomes have been found to contain genes whose products interact with proteins that play a central role in regulating cell survival.

Studies have shown that the interaction of herpes simplex virus 1 (HSV-1) with cells results in programmed cell death and that the virus has evolved mechanisms that block apoptosis whether it is induced by viral gene products or by exogenous agents. Specifically, wild-type HSV does not induce apoptosis, and infection with wild-type virus blocks apoptosis induced by osmotic or thermal shock or by Fas ligand (Koyama et al., 1997; Sieg et al., 1996; Galvan et al., 1998; Leopardi et al., 1996; Galvan et al., 1999; Aubert et al., 1999; Jerome et al., 1999). A number of HSV-1 mutants have been reported to induce apoptosis. These include mutants lacking the infected cell protein numbers 4 (ICP4) or 27 (ICP27) (Leopardi et al., 1996; DeLuca et al., 1985; Auber et al., 1999), two regulatory proteins expressed immediately after infection, a mutant lacking glycoprotein D (Zhou et al., 2000), and a mutant carrying a temperature sensitive mutation that blocks the release of viral DNA from capsids at nuclear pores in cells infected and maintained at the nonpermissive temperatures (Galvan et al., 1998; Baeterson et al., 1983). Detailed analyses of the mutant d120, from which both copies of the gene encoding ICP4 had been deleted, revealed that the virus induces apoptosis in all of the cell lines tested, but that the mechanisms by which the virus induces apoptosis is cell-type dependent (Galvan et al., 1999). In HEp-2 cells, the d120 mutant caused the translocation of cytochrome C from mitochondria, activation of caspase 3 and fragmentation of cellular DNA (Galvan et al., 1999; Galvan et al., 2000). Apoptosis was blocked in a HEp-2-derived cell line that overexpressed Bcl-2 (Galvan et al., 2000).

Earlier studies have also reported that d120 rescueants in which the deleted gene encoding ICP4 was repaired continued to induce apoptosis, but that DNA fragments sharing the U_{S}3 gene blocked apoptosis (Leopardi et al., 1997). Other laboratories have since confirmed that the U_{S}3 protein kinase contributes to HSV-mediated protection from a variety of exogenous apoptotic inducers (Jerome et al., 1999; Asano et al., 1999; Asano et al., 2000; Hata et al., 1999). In a recent study, the inventors have shown that the U_{S}3 protein kinase blocked d120 induced apoptosis at a premitochondria stage, and that activation of caspase 3 could be blocked by the U_{S}3 protein kinase expressed as late as 6 to 9 hrs after infection of HEp-2 cells with the d120 mutant (Munger et al., 2001).

Programmed cell death is triggered by several factors and may take various forms. For example, the synthesis of double-stranded RNA activates kinases which phosphorylate the α subunit of eIF-2 and completely turn off protein synthesis (Sarre, 1989). Ultimately, activation of metabolic pathways causes a pattern of morphological, biochemical, and molecular changes which result in cell death without spillage of cellular constituents which would result in an inflammatory response detrimental to the host (Wyllie et al., 1980.).

Apoptotic cell death is commonly observed during embryogenesis and organ involution and in the natural death of terminally differentiated cells at the end of their life span. Most viruses which induce either the shut-off of protein synthesis or apoptosis also have evolved mechanisms which block host responses and enable them to replicate in their hosts (Shen and Shenk, 1995). Among the best-known examples of viral gene products which block apoptosis are the adenovirus E1B Mᵣ19,000 protein (Rao, et al, 1992.), vaccinia CrmA protein (Ray, et al.), simian virus 40 (SV40) T antigen (McCarthy, et al., 1994), human papillomavirus No. 16 (HPV 16) E6 protein (Pan and Griep, 1994), Epstein-Barr virus BHRF1 protein (Henderson, et al, 1993) and human cytomegalovirus IE1 and IE2 gene products (Zhu, et al., 1995). Herpes simplex virus 1 (HSV-1) encodes a protein, γ₁34.5, which blocks the phosphorylation of eIF-2α (Chou and Roizman, 1992).

The utility of proteins that are capable of inhibiting apoptosis are manifold. First, such proteins, or their corresponding genes, may be used to immortalize cell lines that otherwise would perish during culture. This makes possible not only the study of these cells, but also presents the option of growing these cells in large numbers in order to isolate protein species therefrom. Second, the identification of inhibitors of apoptosis and their function permits the possible intervention, in a clinical setting, when these proteins are interfering with normal programmed cell death, or apoptosis. This may be accomplished by providing an inhibitor or an antisense nucleic acid that interferes with the expression of a protein that interferes with apoptosis.

Furthermore, the identification of proteins that promote apoptosis is also beneficial. Cells that are diseased or infected, either in vitro or in vivo, can be induced to undergo apoptosis. A therapeutic development may be achieved either by inducing such a cell in vivo to undergo apoptosis or by studying such a cell that can be induced to undergo apoptosis in vitro. Cells in vitro can be used to screen for apoptosis inhibitors or inducers. Apoptosis has clinical relevance. Cells that are diseased or infected can be targeted for apoptosis as a therapeutic treatment. For example, the death of cancer, tumor, or other hyperproliferative cells can be beneficial, as could the induced cell death of cells infected with pathogens or cells involved in autoimmune conditions. Furthermore, inhibition of apoptosis has clinical significance too. Because apoptosis has been implicated in atrophy following stroke and heart attack, prevention of apoptosis with respect to those particular conditions could be desirable. Other conditions or diseases involving apoptosis include neurodegenerative disorders such as Alzheimer's disease, Parkinson's disease, amyotrophic lateral sclerosis, retinitis pigmentosa, and cerebellar degeneration, and myelodysplastic syndromes such as aplastic anemia and ischemic injury mentioned above. Thus, both inhibitors and inducers of apoptosis continue to be pursued for scientific and, ultimately, medical purposes.

Thus, the identification of proteins having these activities and uses provide important new tools for those working in this arena.

### SUMMARY OF THE INVENTION

The present invention takes advantage of the observation that herpesviral protein U_{S}3 inhibits apoptosis in a cell by inhibiting the pro-apoptotic protein BAD. Thus, the present invention concerns methods and compositions involving the modulation of BAD activity so as to effect induction or inhibition of apoptosis. The methods and compositions relate to a variety of compounds, including U_{S}3 peptides, polypeptides, and nucleic acids; BAD peptides, polypeptides, and nucleic acids; modulators of U_{S}3 or BAD activity; and, modulators of apoptosis. The methods employ compounds that induce apoptosis such as BAD peptides and polypeptides, inhibitors of U_{S}3, including inhibitors that block or impede U_{S}3 from inhibiting BAD polypeptide—such as BAD peptides or polypeptides that compete with endogenous BAD polypeptide—and modulators of BAD polypeptide that improve or make available the activity of endogenous BAD polypeptide, such as phosphatases. Methods of the invention also involve compounds that inhibit apoptosis. Such compounds include U_{S}3 peptides and polypeptides; inhibitors of BAD polypeptide, such as kinases, 14-3-3 proteins that sequester BAD, and other modulators that alter its pro-apoptotic activities.

In some embodiments, the present invention involves methods for inducing apoptosis in a cell. The cell may or may not be infected with a virus, though in some cases the cell is infected with a herpesvirus, such as herpes simplex virus. It is contemplated that the cell may be infected with any herpesvirus. Human cells may be infected with herpes simplex virus (HSV) type 1, HSV-2, varicella zoster virus, cytomegalovirus (CMV), Epstein-Barr virus, human herpesvirus (HHV) 6, HHV 7, or HHV 8. It is contemplated that methods and examples discussed with respect to one herpesvirus, such as HSV-1, may be applied with respect to other herpesviruses to the extent there is homology among family members. Such methods involve administering to a cell a composition comprising an agent or compound that modulates BAD activity. BAD is a polypeptide of multiple isoforms, which have pro-apoptotic activity.

In any methods of the invention, the term “BAD polypeptide” refers to any BAD polypeptide isoform unless otherwise specified. Human BAD peptides and polypeptides are contemplated for use in the present invention. Also contemplated for use is any BAD peptide or polypeptide from a mammal, such as murine BAD. It is contemplated that murine BAD is interchangeable with human BAD for many embodiments of the invention. The human BAD cDNA sequence is provided as SEQ ID NO: 1 and the corresponding amino acid sequence is provided in SEQ ID NO: 2. The murine BAD cDNA sequence is provided as SEQ ID NO: 3, and the polypeptide sequence is provided as SEQ ID NO: 4. In some embodiments of the invention, an agent that inhibits a herpesviral U_{S}3 polypeptide from modulating BAD activity is administered to the cell, in some cases, in an amount effective to inhibit U_{S}3 modulation of BAD. A herpesviral U_{S}3 refers to a U_{S}3 or homologous protein or peptide from herpes simplex virus (HSV) type 1 (SEQ ID NO: 6), HSV-2 (SEQ ID NO: 7), varicella zoster virus (SEQ ID NO: 8), bovine herpesvirus 1 (SEQ ID NO: 9), equine herpesvirus 1 (SEQ ID NO: 10), equine herpesvirus 4 (SEQ ID NO: 11), galid herpesvirus 1 (SEQ ID NO: 12), galid herpesvirus 2 (SEQ ID NO: 13), galid herpesvirus 3 (SEQ ID NO: 14), cercopithecine herpesvirus 7 (SEQ ID NO: 15), cercopithecine herpesvirus 9 (SEQ ID NO: 16), simian herpesvirus B (SEQ ID NO: 17), infectious laryngotracheitix virus (SEQ ID NO: 18), canine herpesvirus (SEQ ID NO: 19), or suid virus 1 (SEQ ID NO: 20). The other human herpesviruses may also be employed in methods of the invention; they include: cytomegalovirus (CMV), Epstein-Barr virus, human herpesvirus (HHV) 6, HHV 7, or HHV 8. As used herein, “modulation” refers to an alteration of some characteristic of a protein or molecule, such as its activity. In some embodiments, activity is reduced or decreased or inhibited. In other embodiments, modulation of activity constitutes increasing, raising, promoting, or reducing activity. In the context of the present invention, an agent that inhibits a U_{S}3 polypeptide is one that prevents the U_{S}3 polypeptide from inhibiting BAD activity level, including the ability of U_{S}3 to reduce the protein levels of BAD and to phosphorylate BAD at particular residues.

In some embodiments of the invention, an agent that inhibits BAD modulation by U_{S}3 is a BAD peptide. The BAD peptide, in certain embodiments, may contain one or more amino acid residues that may be phosphorylated. Natural amino acids that may be phosphorylated include serine, threonine, and tyrosine. However, any non-natural amino acid that can be phosphorylated is also contemplated as part of the invention. Amino acids, in the context of particular amino acid sequences, that can be phosphorylated by U_{S}3 are specifically contemplated. The BAD peptide may comprise a sequence of between 4 to 100 contiguous amino acids from SEQ ID NO: 2, which is the amino acid sequence for human BAD. In still further embodiments, the peptide may include a sequence that includes ser136, ser155, or ser112, or a combination thereof. Such peptides with additional BAD amino acid sequence are also contemplated. Peptides comprising a sequence of at least 15, 20, 25, 40, 50, 60, 70, 80, 90, or more contiguous amino acids from SEQ ID NO: 2 or SEQ ID NO: 4. In additional embodiments, peptides may have more than one amino acid that can be phosphorylated.

The invention also includes compositions in which two, three, four, five, or more different BAD peptide sequences are provided. The additional peptide sequences may comprise a sequence of between 4 to 100 contiguous amino acids from SEQ ID NO: 2 or SEQ ID NO: 4 and may have characteristics as described above. For example, the second peptide may include an amino acid that can be phosphorylated, such as one that can be phosphorylated by HSV U_{S}3.

To deliver a composition that contains peptides to a cell, the composition may also comprise one or more lipid molecules, a number of which are disclosed in further detail in this application. Lipid compositions may also be used in the context of the present invention to introduce a nucleic acid into a cell, or other therapeutic agents of the invention.

In some embodiments, an agent that inhibits modulation of BAD by U_{S}3 is a polypeptide, instead of a peptide. In some embodiments, the polypeptide can bind to BAD that may or may not be phosphorylated. In some cases, the polypeptide binds a BAD polypeptide, and such binding involves or affects an amino acid residue that can be phosphorylated, particularly one that can be phosphorylated by U_{S}3. In some cases, the binding may affect the ability of U_{S}3 to inhibit BAD, but it may not affect the pro-apoptotic activity of BAD. Under these circumstances, the agent would prevent or interfere with U_{S}3 so that BAD could induce apoptosis in the cell. The BAD polypeptide may have the amino acid sequence identified in SEQ ID NO: 2 or SEQ ID NO: 4. In some embodiments, the agent binds to, masks, or hides an amino acid residue(s) that is involved in U_{S}3 inhibition of BAD. In some embodiments, the amino acid residue is one that may be post-translationally modified, such as by phosphorylation, by U_{S}3 or other kinases. Such residues include ser136, ser155, or ser112. It is also contemplated that multiple amino acid residues may be involved, including a combination of these residues. Thus, ser136 and ser155 may be included on particular peptides or polypeptides for use as inhibitors of U_{S}3.

In some cases, polypeptides of the invention may be antibodies, such as a monoclonal antibody, polyclonal antibody, single chain antibody, a humanized antibody, or a bi-specific antibody.

In additional embodiments, a polypeptide may be employed not to bind BAD but to compete with it. In those cases, methods involve administering to the cell a BAD polypeptide. As discussed above, a BAD polypeptide may comprise at least 100 contiguous amino acids of SEQ ID NO: 2 or SEQ ID NO: 4.

It is further contemplated that, while peptides and polypeptides may be employed in methods of the invention, they may be first provided as a nucleic acid that is transcribed and translated into the desired proteinaceous compound. The nucleic acid may be an expression construct. In some embodiments an expression construct is a viral vector, such as an adenovirus, adeno-associated virus, herpesvirus, lentivirus, retrovirus, vaccinia virus, or other viruses vectors employed with respect to gene transfer.

In some embodiments, particularly those involving therapeutic or preventative therapies, methods concerning infected cells may also include administering to the cells an antiviral agent. Antiviral agents against herpesvirus infection are specifically included, such as famcyclovir, valacyclovir, or acyclovir. Antiviral agents may be administered before, during, or after other therapeutic compositions—those modulating apoptosis—are provided to cells. Therapeutic methods of the invention are specifically contemplated to be of use on patients, particularly human patients.

Methods for treating a patient infected with herpes simplex virus are part of the present invention. Methods including administering to the patient an effective amount of a composition that includes a peptide comprising a sequence comprising between 4 to 100 amino acids of SEQ ID NO: 2 or SEQ ID NO: 4. Peptide compositions discussed above are contemplated for use with any treatment methods disclosed herein, including methods for treating patients infected with viruses such as herpesviruses. Also included in such methods are other modulators of apoptosis, such as modulators of BAD or U_{S}3.

Other methods of the invention, which have therapeutic and research uses, involve methods for blocking BAD-induced apoptosis of a cell. In some embodiments, such methods involve providing a U_{S}3 polypeptide to a cell suspected of undergoing BAD-induced apoptosis. The phrase “BAD-induced apoptosis” refers to apoptosis induced by or involving BAD. Of course, one way of identifying BAD-induced apoptosis is to determine whether BAD is expressed in such a cell. An “effective amount” of a U_{S}3 polypeptide may be provided to the cell so as to prevent the cell from undergoing apoptosis. It is contemplated that any herpesviral U_{S}3 may be implemented in methods of the invention. Specifically contemplated are U_{S}3 polypeptide sequences comprising all or part of SEQ ID NO: 6, 7, 8, 9, 10, 11, 12, 13, 14, 15, 16, 17, 18, 19, and 20. As discussed above and in further detail elsewhere, the U_{S}3 polypeptide may be provided to a cell by introducing into the cell an expression construct that includes a nucleic acid sequence encoding all or part of a U_{S}3 polypeptide or peptide. The expression construct may contain a nucleic acid sequence of SEQ ID NO: 5. Any nucleic acid sequence may be under the transcriptional control of a promoter active in eukaryotic cells. In some embodiments, the promoter is a tetracycline controlled promoter. Expression constructs may also include a selectable marker.

Screening methods are also provided by the invention. Embodiments discussed with respect to therapeutic methods may be implemented with respect to screening methods as well, and vice versa.

In some embodiments, screening methods can be used to identify a therapeutic agent to modulate apoptosis comprising a) contacting a BAD peptide or polypeptide with a candidate compound; and b) assaying the compound for an ability to modulate the activity of the BAD peptide or polypeptide. As discussed earlier, a modulator of activity is one that may affect activity, directly or indirectly, by inter alia reducing, inhibiting, retarding, promoting, inducing or increasing it. While the BAD polypeptide or peptide may have multiple activities, many of the embodiments of the invention concern its apoptosis ability, that is, the ability of BAD to promote apoptosis in a cell. A compound that increases or promotes the activity, directly or indirectly, of the BAD peptide or polypeptide capable of inducing apoptosis is a possible therapeutic agent to promote apoptosis, if an increase in apoptosis activity is detected. A compound that reduces the activity, directly or indirectly, of the BAD peptide or polypeptide capable of inducing apoptosis, is a possible therapeutic agent to inhibit apoptosis if a reduction in apoptosis activity is detected. Screening methods of the invention may be performed in vitro, in cells, or in organisms. Factors contributing to a reduction in activity include, but are not limited to, reduction in BAD transcript level, reduction in BAD peptide/polypeptide level, sequestering of BAD transcripts, peptides, or polypeptides, increase in kinase activity, increase in post-translational modification of BAD peptides/polypeptides, decrease in phosphatase activity on BAD peptides/polypeptides, binding to the BAD peptide or polypeptide so as to reduce any activity of BAD, such as its ability to bind Bcl-X_{L}. Factors contributing to an increase or promotion in activity include, but are not limited to, an increase in BAD transcript or peptide/polypeptide level, localization of BAD-encoding nucleic acids or BAD peptides/polypeptides, inhibition of BAD inhibitors such as kinases, increase in phosphatase activity on BAD peptides/polypeptides, and binding to proteins or polypeptides that bind and sequester BAD peptides/polypeptides, such as 14-3-3 proteins. Comparisons of these factors between BAD peptides/polypeptides exposed to a candidate compound and those not exposed to the candidate compound can be done to determine the effect of the candidate compound on a particular characteristic or activity of BAD. In some embodiments, a BAD peptide or polypeptide may be contacted with a U_{S}3 under conditions that allow U_{S}3 to inhibit BAD-induced apoptosis. The contact may occur before or after the BAD peptide/polypeptide is incubated with the candidate compound. The effect of the candidate compound on BAD with respect to U_{S}3 inhibition of BAD may be evaluated to identify a compound that relieves U_{S}3 inhibition or reduces the inhibition in any way. Such a compound could be used to promote apoptosis.

Characteristics of apoptosis can be assayed or evaluated in methods of the invention. Assays may involve measuring or observing cell shrinkage, nuclear chromatin condensation and margination, or DNA fragmentation. Alternatively, caspase or procaspases may be evaluated to qualify or quantify apoptosis. The cleavage of a procaspase, its activity based on the amount of its substrate is used or the amount of a product it makes based on the substrate conversion, or the amount of cytochrome c released from the mitochondria may be evaluated. Automation can be done with procaspase cleavage or substrate cleavage.

In some embodiments of the screening methods, also included is a step concerning assaying the ability of the BAD peptide or polypeptide to promote apoptosis after contacting the BAD peptide or polypeptide with the candidate compound. While in other embodiments a BAD peptide or polypeptide activity is evaluated based on amount of BAD-encoding nucleic acids or BAD peptide or polypeptide. In still further embodiments, BAD activity may be evaluated by assaying for post-translation modification of BAD. This may involve identifying whether BAD is modified at all, to what extent it is modified, or at which location(s) it is modified, such as at any of the phosphorylated residues of BAD discussed throughout this application. Alternatively, BAD peptide or polypeptide activity may be evaluated based on the ability to specifically bind a substrate or binding protein, such as Bcl-X_{L} or 14-3-3. Other methods of the invention concern identifying binding sites on BAD. Such methods may involve mutating BAD, by implementing deletions or substitutions of amino acids, and assaying for binding activity or by using peptides of BAD, assaying the peptides for binding activity, and mapping the sites. Any of the proteinaceous compounds that binds to BAD may be employed to map the binding or active sites of BAD.

BAD peptides or polypeptides for use with methods of the invention are disclosed above. In some embodiments, BAD peptides are employed, while in other embodiments, BAD polypeptides are used.

Candidate compounds that may be tested in screening methods of the invention include peptides, polypeptides—including antibodies—small molecules, and peptide mimetics. Candidate compounds may also be comprised in combinatorial or small molecule libraries. They also may be contained in expression libraries or implemented using high throughput screening methods known to those of ordinary skill in the art. Bioinformatics, computer modeling, or DNA-chip technology may also be employed in methods of the invention.

Apoptosis modulators identified by screening methods of the invention are contemplated as part of the present invention. Such methods include a) contacting a BAD peptide or polypeptide with a candidate compound; and b) assaying the compound for an ability to modulate the activity of the BAD peptide or polypeptide, wherein the compound modulates the activity of the BAD peptide or polypeptide. In further embodiments, the process further includes c) incubating the BAD peptide or polypeptide with a U_{S}3 polypeptide; and d) comparing the BAD peptide or polypeptide after being contacted with the candidate compound to a second BAD peptide or polypeptide not contacted with the candidate compound. Apoptosis modulators may be apoptosis inhibitors or apoptosis inducers, as the invention provides materials to identify either or both.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Diagrammatic representation of the HSV-1 genome showing the location of the α4 and U_{L}29 genes encoding ICP4 and ICP8, respectively. The reiterated sequences (open rectangles) flanking the unique short (U_{S}) and unique long (U_{L}) sequences (thin lines) and the location and direction of genes are as shown. Because the α4 gene maps within inverted repeats flanking the U_{L}, it is present in two copies per genome. The hatched lines within the rectangles indicates the position of the sequences deleted from the d120 mutant (DeLuca et al., 1985).

FIG. 2. BHK-C13 cells were infected at a multiplicity of 10 pfu/cell with the indicated viruses. At 13 hr post-infection cells were incubated in phosphate-free medium for 1 hr and then labeled with 32 Pi for 4 hr. Cell lysates were electrophoretically separated in SDS polyacrylamide gels and electrically transferred onto nitrocellulose filters. Filters were exposed to film or probed with a U_{L}34 antibody.

FIG. 3. The effect of baculovirus-mediated gene expression on DEVDase activity in cells. Replicate 25 cm² flask cultures of HEp-2 cells were either mock infected or infected with 10 or 20 PFU of Bac-WT or Bac-U_{S}3 or 0.5, 2.5, 5.0 PFU of Bac-BAD per cell. The cells were harvested at 18 hrs after Bac-BAD infection and assayed for DEVDase activity colorimetrically at 405 nm as described in Example 6. The results are expressed as fold increase in activity over that of mock-infected cells.

FIG. 4. The effect of U_{S}3 protein kinase on DEVDase activity induced by BAD. Replicate HEp-2 cell cultures were exposed to 5 PFU of Bac-BAD per cell 6.5 hrs after infection of the cells with 20 PFU of either Bac-WT or Bac-U_{S}3 per cell. The cells were harvested and processed as described in the legend to FIG. 3. The results are expressed as fold increase in DEVDase activity over that of mock-infected cells.

### DETAILED DESCRIPTION OF THE INVENTION

The identification of novel proteins having apoptotic activities and uses will provide important new tools to develop therapeutic and preventative compounds and methods of HSV infection, as well as other diseases and conditions in which apoptosis can or may be involved or implemented to effect beneficial treatment. The present invention provides methods for the use of U_{S}3 gene and its gene product as inhibitors of apoptosis, as well as methods concerning the induction and inhibition of apoptosis through the BAD polypeptide.

The herpes simplex virus 1 (HSV-1) mutant lacking the major regulatory gene designated α4 induces apoptosis, whereas in cells infected with wild-type virus do not exhibit apoptosis (Leopardi and Roizman, 1996). It has been reported that wild-type virus blocked apoptosis induced by thermal shock. Recent studies by Koyama and Miwa (1997), and also in the inventors laboratory, demonstrated that the virus also blocks the induction of apoptosis by osmotic shock. These studies suggested that a functional α4 gene was necessary to block apoptosis but did not address the question of sufficiency. This led to several studies that included the rescue of the deleted α4 genes in the mutant virus.

A mutant lacking the α4 gene, HSV-1(KOS)d120, was discovered, due to lack of complementation of the α4 gene, to have a secondary mutation in the gene U_{S}3, encoding a protein kinase. Thus, a functional U_{S}3 is required in order to block apoptosis. This important finding was exploited to identify the mechanism behind the inhibition of apoptosis, as well as provide methods concerning the induction of apoptosis, as described herein below. Further, this invention allows for the provision of methods for the use of agents that inhibit U_{S}3 function and expression in order to induce apoptosis in HSV-infected cells, and for the identification of agents that inhibit the apoptosis-inhibiting function of ICP4. These and other related aspects of the present invention are described in further detail herein below. U.S. patent application Ser. Nos. 08/843,659 and 09/837,861 discuss some of the observations on which the present application is based; these applications are specifically incorporated by reference herein.

Furthermore, the present invention involves the observation that U_{S}3 from a herpesvirus prevents apoptosis by modulating the pro-apoptotic protein BAD. Methods for inducing apoptosis by administering BAD or for inducing apoptosis in a cell inhibited from undergoing apoptosis by U_{S}3 are also provided. Screens for identifying BAD modulators are also described.

### I. HERPESVIRUSES

Herpesviruses constitute a family (Herpesviridae) of DNA viruses with genomes between 120 and 235 kilobases. They have linear, double-stranded DNA and an icosahedral shape and are enveloped. Human herpesviruses (HHV) include Herpes Simplex Virus types 1 and 2 (HHV-1 and HHV-2; commonly known as HSV-1 and HSV-2); Varicella zoster (known as VZV or HHV-3), which causes chicken pox; Epstein Bar virus (known as EBV or HHV-4); human cytomegalovirus (known as HCMV or HHV-5); human herpesvirus 6 (HHV-6); human herpesvirus 7 (HHV-7); and Kaposi's sarcoma herpesvirus or human herpesvirus 8 (KSHV or HHV-8). They are classified into one of three classes—alpha, beta, and gamma—based upon of tissue tropism, pathogenicity, and behavior.

Alpha herpesviruses are typically fast replicating; HSV-1 and -2, and Varicella zoster virus are α herpesviruses. Beta herpesviruses are slow replicaters and they include cytomegalovirus and human herpesvirus-6 and -7. Gamma herpesviruses poorly replicate and readily transform cells. Epstein-Barr Virus, which transforms B-cells and causes infectious mononucleosis, and the human herpesvirus-8 are gamma herpesviruses. While there are significant differences among the herpesvirus family members, there is notable homology among the protein sequences of cognate polypeptides. The U_{S}3 gene and encoded U_{S}3 polypeptide represents such a polypeptide that has considerable homology and identity among the human herpesviruses. Thus, methods of the present invention are directed to herpesviruses and, in some embodiments, mores specifically to individual herpesviruses. It is contemplated that methods and compositions with respect to the U_{S}3 from HSV-1 may be implemented with respect to other herpesvirus U_{S}3 genes and polypeptides. The U_{S}3 gene and polypeptide sequences for the following herpesvirus members are provided herein. HSV-1 gene and polypeptide sequences are provided as SEQ ID NO: 5 and 6, respectively. The polypeptide sequences of other U_{S}3 polypeptides include: HSV-2 (SEQ ID NO: 7), varicella zoster virus (SEQ ID NO: 8), bovine herpesvirus 1 (SEQ ID NO: 9), equine herpesvirus 1 (SEQ ID NO: 10), equine herpesvirus 4 (SEQ ID NO: 11), galid herpesvirus 1 (SEQ ID NO: 12), galid herpesvirus 2 (SEQ ID NO: 13), galid herpesvirus 3 (SEQ ID NO: 14), cercopithecine herpesvirus 7 (SEQ ID NO: 15), cercopithecine herpesvirus 9 (SEQ ID NO: 16), simian herpesvirus B (SEQ ID NO: 17), infectious laryngotracheitix virus (SEQ ID NO: 18), canine herpesvirus (SEQ ID NO: 19), or suid virus 1 (SEQ ID NO: 20).

A. Herpes Simplex Viruses

Herpes simplex viruses, designated with subtypes 1 and 2, are enveloped viruses that are among the most common infectious agents encountered by humans, infecting millions of human subjects worldwide. These viruses cause a broad spectrum of disease which ranges from relatively insignificant to severe and life-threatening. The clinical outcome of herpes infections is dependent upon early diagnosis and prompt initiation of antiviral therapy. Despite some successful efforts in treating HSV infectious, dermal and epidermal lesion often recur, and HSV infections of neonates and infections of the brain are associated with high morbidity and mortality.

The large, complex, double-stranded DNA genome encodes dozens of different gene products, some of which derive from spliced transcripts. In addition to virion and envelope structural components, the virus encodes numerous other proteins including a protease, a ribonucleotides reductase, a DNA polymerase, a ssDNA binding protein, a helicase/primase, a DNA dependent ATPase, a dUTPase and others.

HSV genes form several groups whose expression is coordinately regulated and sequentially ordered in a cascade fashion (Honess and Roizman, 1974; Honess and Roizman 1975; Roizman and Sears, 1996). The expression of α genes, the first set of genes to be expressed after infection, is enhanced by the virion protein number 16, or α-transinducing factor (Post et al., 1981; Batterson and Roizman, 1983; Campbell, et al., 1984). The expression of β genes requires functional α gene products, most notably ICP4, which is encoded by the α4 gene (DeLuca et al., 1985). γ genes, a heterogeneous group of genes encoding largely virion structural proteins, require the onset of viral DNA synthesis for optimal expression (Holland et al, 1980).

In line with the complexity of the genome, the life cycle of HSV is quite involved. In addition to the lytic cycle, which results in synthesis of virus particles and, eventually, cell death, the virus has the capability to enter a latent state in which the genome is maintained in neural ganglia until some as of yet undefined signal triggers a recurrence of the lytic cycle.

HSV-1 replication, defined by accumulation of infectious virus, lasts approximately 18-24 hrs although viral protein synthesis can last much longer. The replication of HSV is accompanied by the development of cytopathic effects. In cells overexpressing Bcl-2, the development of cytopathic effects is delayed without significant effect on viral replication (Galvan et al., 2000). These results suggest that cytopathic effects at the end of the replicative cycle reflect injuries to the cell resulting from pro-apoptotic events. These pro-apoptotic events most likely are not specifically targeted by the virus to be blocked inasmuch as they are not detrimental to viral replication as they occur very late during infection. Alternatively, some pro-apoptotic events may benefit viral replication. If such were the case, the virus would dictate the cellular apoptotic environment by blocking apoptotic events that are detrimental to viral replication and by allowing apoptotic events that benefit viral replication. The available data suggest that the virus has evolved a number of inducers of apoptosis and very likely, each is blocked by specific gene products. A number of different HSV-1 gene products have been reported to inhibit apoptosis; among them U_{S}3, gD and gJ (Leopardi et al., 1996; Jerome et al., 1999; Zhou et al., 2000). It must be stressed, however, that in the normal course of wild-type virus replication, i.e., at 18-24 hrs, late apoptotic manifestations such as caspase 3 activation and DNA fragmentation are not detected and as noted in the introduction, wild-type virus protects cells from these apoptotic manifestations induced by exogenous agents.

HSV-1 encodes two well characterized protein kinases. The U_{L}13 protein kinase has been shown to mediate the phosphorylation of a large number of viral and cellular proteins. In contrast, the U_{S}3 protein kinase appears to have a narrow, less defined range of substrates. Its functions in the course of viral infection has always been somewhat of a puzzle: although a virus deleted for the U_{S}3 ORF is growth impaired in mice (Purves et al., 1987), the gene is not essential for viral replication in tissue culture although the cytopathic effects of the deletion mutant are very different from those of wild-type virus. By light microscopy, the cells have a crenated appearance but exhibit no evidence of classical apoptosis resulting in activation of caspases or degradation of cellular DNA.

1. The U_{S}3 Polypeptide

HSV encodes two protein kinases expressed by the genes U_{S}3 and U_{L}13, respectively (reviewed in Roizman and Sears, 1996). Whereas U_{L}13 is packaged in the virion, U_{S}3 is not. Not all substrates of the U_{S}3 are known (Purves et al., 1986; 1987; Leader et al., 1991). The major substrate of U_{S}3 protein kinase is an intrinsic membrane protein exposed on the surface of infected cells and encoded by the U_{L}34 gene (Purves et al., 1991; 1992). The U_{L}34 is phosphorylated by more than one kinase (Purves 1991, 1992). In the absence of U_{S}3 protein kinase, the U_{L}34 protein has an apparent Mᵣ of 33,000 and associates with several cellular phosphoproteins, whereas in wild-type infected cells U_{L}34 has an apparent Mᵣ of 30,000 and does not exhibit an association with the host proteins (Purves et al., 1992).

The U_{S}3 protein kinase phosphorylates threonine/serine in the consensus sequence RRR-R/X-S/T-R/Y (SEQ ID NO: 22; SEQ ID NO: 23; SEQ ID NO: 24; SEQ ID NO: 25) (Purves et al., 1986; Leader et al., 1991). In the case of U_{L}34, this was verified by mutagenesis of the threonine codon in the sequence encoding RRRRTRRSRE (SEQ ID NO: 21) (Purves et al., 1991; 1992). The apparent Mᵣ of the mutant U_{L}34 protein was 33,000, that is, corresponding to the apparent Mᵣ of the U_{L}34 protein in cells infected with a virus lacking the U_{S}3 gene.

U_{S}3 blocks apoptosis by phosphorylating one or more proteins; BAD polypeptide is one such protein. As stated above, for ICP4 to be functional it needs to be phosphorylated, this leads the inventors to believe that one of the substrates of U_{S}3 protein kinase is ICP4 itself, among other, as yet unidentified, phosphoproteins.

The involvement of U_{S}3 protein kinase in the blocking of apoptosis induced by infection, thermal or osmotic shocks suggests that HSV differs from other viruses in the mechanism by which it blocks the apoptosis as a result to a host response to infection. Thus, U_{S}3 may be used to control or modulate apoptosis independent of viral infection.

Accordingly, the present invention takes advantage of the mechanism by which the protein kinase U_{S}3 is able to inhibit apoptosis by modifying the pro-apoptotic protein BAD, either by reducing its expression level or availability or its phosphorylation. Apoptosis, or programmed cell death, is characterized by certain cellular events, including nuclear condensation, DNA fragmentation, cytoplasmic membrane blebbing and, ultimately, irreversible cell death. Apoptosis is an energy-dependent event. For the purposes of this application, apoptosis will be defined as inducing one or more of these events. Thus, use of the term “U_{S}3” in this application encompasses polypeptides having all or part of the U_{S}3 polypeptide sequence and the anti-apoptosis function of U_{S}3. These need not be wild-type U_{S}3.

This functional attribute is manifested, for example, in U_{S}3's ability to protect cells from apoptosis triggered by modification of the BAD polypeptide. The term “modification” with respect to BAD refers to any alteration, direct or indirect, on BAD activity or function, including alterations on BAD transcript or BAD polypeptide such as their expression levels, in transcript or polypeptide stability or half-life, in transcript or polypeptide cellular localization, in transcript or polypeptide availability, in post-translational additions to amino acid residues (including phosphorylation) on the polypeptide, or on the availability or accessibility to a BAD active site or binding site. This observation permits utilization of U_{S}3 or ICP4 alone or in combination with each other, in a number of ways that could not have been predicted from the prior art. For example, according to the present invention, the production of HSV vectors or recombinant proteins from HSV vectors can be enhanced by increasing the apoptosis inhibiting function of U_{S}3 or ICP4 or both. When cells are infected with HSV, premature cell death can limit the titer of virus produced or the amount of recombinant protein synthesized. Similarly, U_{S}3 and ICP4 may prolong the life of the cells expressing human or animal genes introduced into cells by viral vectors in order to correct a genetic defect. If the cell can be sustained longer, the titer of the virus stocks and the amount of protein should increase.

2. The BAD Pro-Apoptotic Polypeptide

Members of the Bcl-2 family of proteins regulate the execution of programmed cell death. The members of this family can be functionally separated into apoptotic antagonists, including Bcl-2, Bcl-X_{L} and Bcl-w, and apoptotic agonists, such as BAD, BID, and BAX. These key apoptotic regulators mediate their pro- or anti-apoptotic signals through their relative abundance, subcellular localization and posttranslational modifications.

As used herein, the term “apoptosis” refers to the physiological process known as programmed cell death. This process is a morphologically and biochemically distinct form of cell death that regulates cell turnover under normal physiological conditions. The morphological features include an orchestrated sequence of changes that include cell shrinkage, chromatin condensation, nuclear segmentation and eventual cellular disintegration into discrete membrane-bound apoptotic bodies. The biochemical features include, for example, internucleosomal cleavage of cellular DNA and the activation of ICE/Ced-3 family of proteases. The term “apoptosis” is used here synonymously with the phrase “programmed cell death.” These terms are intended to be consistent with their use as they are known and used by those skilled in the art.

Pro- and anti-apoptotic family members are capable of dimerizing through the three Bcl-2 homology domains (BH1, BH2, and BH3) apparently titering out each other's function (Oltvai et al., 1993; Chittenden et al., 1995; Yin et al., 1994). Specifically, BH1, BH2 and BH3 domains form a hydrophobic cleft to which the BH3 domain can bind (Sattler et al., 1997). Some pro-apoptotic Bcl-2 family members, such as BAD, contain only the BH3 domain which is essential for binding to anti-apoptotic family members, such as Bcl-2 and Bcl-X_{L} and for their pro-apoptotic function (Chittenden et al., 1995).

Cell survival signals block BAD from inducing apoptosis by phosphorylation (Franke et al., 1997). Some of these signals activate phosphatidylinositol (PI) 3-kinase with subsequent activation of Akt, which phosphorylates BAD at serine 136 (del Peso et al., 1997; Scheid et al., 1998). Survival signals also promote the activation of RSK and PKA which have both been shown to phosphorylate BAD at ser112 (Tan et al., 1999; Harada et al., 1999). Phosphorylation of BAD at ser112 and ser136 has been demonstrated to abrogate its pro-apoptotic activity by promoting its association with 14-3-3 proteins which sequester phosphorylated BAD, thereby preventing its localization to the mitochondria and association with BCl-X_{L} (Harada et al., 1999; Pastorino et al., 1998; Zha et al., 1996; Hsu et al., 1997). Additionally, phosphorylation of BAD at serine 155 disrupts its interaction with BCl-X_{L} (Zhou et al., 2000; Tan et al., 2000; Datta et al., 2000). Conversely, activation of BAD appears to be carried out by phosphatases. Thus two different phosphatases, calcineurin and PP1α dephosphorylate BAD at serine 112 and 136, thereby releasing BAD from 14-3-3-proteins, stimulating its binding to Bcl-2/Bcl-X_{L}, and ultimately leading to cytochrome c release, caspase activation and apoptosis (Salomoni et al., 2000; Wang et al., 1999). Recently it has been reported that death receptor engagement induces the caspase mediated cleavage of BAD, yielding an Mᵣ 15,000 truncated protein that is a more potent inducer of apoptosis than the full length BAD (Condreay et al., 1999). The human BAD cDNA and protein sequences are provided as SEQ ID NO: 1 and 2, respectively, while the murine cDNA and protein sequences are provided as SEQ ID NO: 3 and SEQ ID NO: 4, respectively. Information about BAD can be found in U.S. Pat. No. 5,965,703, which is specifically incorporated by reference. The sequence for the murine BAD cDNA and protein sequences are readily available to those of ordinary skill in the art.

### II. PROTEINACEOUS COMPOSITIONS

In certain embodiments, the present invention concerns compositions comprising at least one proteinaceous molecule. The proteinaceous molecule may be a modulator of apoptosis through U_{S}3 or BAD or it may be used as a candidate substance to be screened as a modulator of BAD activity or of U_{S}3 activity. The proteinaceous molecule may also be used, for example, in a pharmaceutical composition for the delivery of a therapeutic agent or as part of a screening assay to identify apoptosis modulators. As used herein, a “proteinaceous molecule,” “proteinaceous composition,” “proteinaceous compound,” “proteinaceous chain” or “proteinaceous material” generally refers, but is not limited to, a protein of greater than about 200 amino acids or the full length endogenous sequence translated from a gene; a polypeptide of greater than about 100 amino acids; and/or a peptide of from about 3 to about 100 amino acids. All the “proteinaceous” terms described above may be used interchangeably herein.

In certain embodiments, the size of the at least one proteinaceous molecule may comprise, but is not limited to, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 or greater amino molecule residues, and any range derivable therein. Furthermore, such proteinaceous molecules may include 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750,2000, 2250, 2500 or more contiguous amino acid residues from SEQ ID NO: 2, SEQ ID NO: 4, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, or SEQ ID NO: 25.

As used herein, an “amino molecule” refers to any amino acid, amino acid derivative or amino acid mimic as would be known to one of ordinary skill in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

Accordingly, the term “proteinaceous composition” encompasses amino molecule sequences comprising at least one of the 20 common amino acids in naturally synthesized proteins, or at least one modified or unusual amino acid, including but not limited to those shown on Table 1 below.

TABLE 1
Modified and Unusual Amino Acids

| Abbr. | Amino Acid |
|---|---|
| Aad | 2-Aminoadipic acid |
| Baad | 3-Aminoadipic acid |
| Bala | β-alanine, β-Amino-propionic |
| | acid |
| Abu | 2-Aminobutyric acid |
| 4Abu | 4-Aminobutyric acid, piperidinic |
| | acid |
| Acp | 6-Aminocaproic acid |
| Ahe | 2-Aminoheptanoic acid |
| Aib | 2-Aminoisobutyric acid |
| Baib | 3-Aminoisobutyric acid |
| Apm | 2-Aminopimelic acid |
| Dbu | 2,4-Diaminobutyric acid |
| Des | Desmosine |
| Dpm | 2,2′-Diaminopimelic acid |
| Dpr | 2,3-Diaminopropionic acid |
| EtGly | N-Ethylglycine |
| EtAsn | N-Ethylasparagine |
| Hyl | Hydroxylysine |
| AHyl | allo-Hydroxylysine |
| 3Hyp | 3-Hydroxyproline |
| 4Hyp | 4-Hydroxyproline |
| Ide | Isodesmosine |
| AIle | allo-Isoleucine |
| MeGly | N-Methylglycine, |
| | sarcosine |
| MeIle | N-Methylisoleucine |
| MeLys | 6-N-Methyllysine |
| MeVal | N-Methylvaline |
| Nva | Norvaline |
| Nle | Norleucine |
| Orn | Ornithine |

In certain embodiments the proteinaceous composition comprises at least one protein, polypeptide or peptide. In further embodiments the proteinaceous composition comprises a biocompatible protein, polypeptide or peptide. As used herein, the term “biocompatible” refers to a substance which produces no significant untoward effects when applied to, or administered to, a given organism according to the methods and amounts described herein. Organisms include, but are not limited to, Such untoward or undesirable effects are those such as significant toxicity or adverse immunological reactions. In preferred embodiments, biocompatible protein, polypeptide or peptide containing compositions will generally be mammalian proteins or peptides or synthetic proteins or peptides each essentially free from toxins, pathogens and harmful immunogens.

Proteinaceous compositions may be made by any technique known to those of skill in the art, including the expression of proteins, polypeptides or peptides through standard molecular biological techniques, the isolation of proteinaceous compounds from natural sources, or the chemical synthesis of proteinaceous materials. The nucleotide and protein, polypeptide and peptide sequences for various genes have been previously disclosed, and may be found at computerized databases known to those of ordinary skill in the art. One such database is the National Center for Biotechnology Information's Genbank and GenPept databases (http://www.ncbi.nlm.nih.gov/). The coding regions for these known genes may be amplified and/or expressed using the techniques disclosed herein or as would be know to those of ordinary skill in the art. Alternatively, various commercial preparations of proteins, polypeptides and peptides are known to those of skill in the art.

In certain embodiments a proteinaceous compound may be purified. Generally, “purified” will refer to a specific protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

It is contemplated that virtually any protein, polypeptide or peptide containing component may be used in the compositions and methods disclosed herein. However, it is preferred that the proteinaceous material is biocompatible. In certain embodiments, it is envisioned that the formation of a more viscous composition will be advantageous in that it will allow the composition to be more precisely or easily applied to the tissue and to be maintained in contact with the tissue throughout the procedure. In such cases, the use of a peptide composition, or more preferably, a polypeptide or protein composition, is contemplated. Ranges of viscosity include, but are not limited to, about 40 to about 100 poise. In certain aspects, a viscosity of about 80 to about 100 poise is preferred.

Proteins and peptides suitable for use in this invention may be autologous proteins or peptides, although the invention is clearly not limited to the use of such autologous proteins. As used herein, the term “autologous protein, polypeptide or peptide” refers to a protein, polypeptide or peptide which is derived or obtained from an organism. Organisms that may be used include, but are not limited to, a bovine, a reptilian, an amphibian, a piscine, a rodent, an avian, a canine, a feline, a fungal, a plant, or a prokaryotic organism, with a selected animal or human subject being preferred. The “autologous protein, polypeptide or peptide” may then be used as a component of a composition intended for application to the selected animal or human subject. In certain aspects, the autologous proteins or peptides are prepared, for example from whole plasma of the selected donor. The plasma is placed in tubes and placed in a freezer at about −80° C. for at least about 12 hours and then centrifuged at about 12,000 times g for about 15 minutes to obtain the precipitate. The precipitate, such as fibrinogen may be stored for up to about one year (Oz, 1990).

A. Antibodies

In certain embodiments, the proteinaceous composition may comprise at least one antibody. Antibodies can be used in the context of the present invention as an apoptosis modulator or in screening assays to identify apoptosis modulators. Furthermore, they may be employed to determine whether a cell is infected with a herpesvirus or whether the cell expresses a particular protein, in order to understood the mechanism by which apoptosis is inhibited in herpesvirus-infected cells or to gain in understanding about how to inhibit or induce apoptosis more generally.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting. The term “antibody” is used to refer to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments, as well as means for preparing and characterizing antibodies, are well known in the art (See, e.g., Antibodies: A Laboratory Manual, Cold Spring Harbor Laboratory, 1988; incorporated herein by reference).

In certain embodiments, the present invention involves antibodies. For example, all or part of a monoclonal, single chain, or humanized antibody may be employed as an apoptosis modulator, for example, an antibody against BAD to either inhibit U_{S}3 from acting on it so as to effect apoptosis or to prevent BAD from promoting apoptosis. Alternatively, other aspects of the invention involve detecting a particular antigen or antigenic region for use in screening methods of the present invention. As detailed above, in addition to antibodies generated against full length proteins, antibodies also may be generated in response to smaller constructs comprising epitopic core regions, including wild-type and mutant epitopes. An epitope is an antigenic determinant. An antigen is any substance that is specifically recognized by an antibody or T-cell receptor. An immunogen is an antigen that induces a specific immune response.

Monoclonal antibodies (mAbs) are recognized to have certain advantages, e.g., reproducibility and large-scale production, and their use is generally preferred. The invention thus provides monoclonal antibodies of the human, murine, monkey, rat, hamster, rabbit and even chicken origin.

The methods for generating monoclonal antibodies (mAbs) generally begin along the same lines as those for preparing polyclonal antibodies. Briefly, a polyclonal antibody may be prepared by immunizing an animal with an immunogenic polypeptide composition in accordance with the present invention and collecting antisera from that immunized animal. Alternatively, in some embodiments of the present invention, serum is collected from persons who may have been exposed to a particular antigen. Exposure to a particular antigen may occur in a work environment, such that those persons have been occupationally exposed to a particular antigen and have developed polyclonal antibodies to a peptide, polypeptide, or protein. In some embodiments of the invention polyclonal serum from occupationally exposed persons is used to identify antigenic regions in the gelonin toxin through the use of immunodetection methods.

A wide range of animal species can be used for the production of antisera. Typically the animal used for production of antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin also can be used as carriers. Means for conjugating a polypeptide to a carrier protein are well known in the art and include glutaraldehyde, m-maleimidobenzoyl-N-hydroxysuccinimide ester, carbodiimide and bis-biazotized benzidine.

As also well known in the art, the immunogenicity of a particular immunogen composition can be enhanced by the use of non-specific stimulators of the immune response, known as adjuvants. Suitable molecule adjuvants include all acceptable immunostimulatory compounds, such as cytokines, toxins or synthetic compositions.

Adjuvants that may be used include IL-1, IL-2, IL-4, IL-7, IL-12, γ-interferon, GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion also is contemplated. MHC antigens may even be used. Exemplary adjuvants include complete Freund's adjuvant (a non-specific stimulator of the immune response containing killed Mycobacterium tuberculosis), incomplete Freund's adjuvants and aluminum hydroxide adjuvant.

In addition to adjuvants, it may be desirable to coadminister biologic response modifiers (BRM), which have been shown to upregulate T cell immunity or downregulate suppressor cell activity. Such BRMs include, but are not limited to, Cimetidine (CIM; 1200 mg/d) (Smith/Kline, PA); low-dose Cyclophosphamide (CYP; 300 mg/m²) (Johnson/Mead, NJ), cytokines such as γ-interferon, IL-2, or IL-12 or genes encoding proteins involved in immune helper functions, such as B-7.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization.

A second, booster injection also may be given. The process of boosting and titering is repeated until a suitable titer is achieved. When a desired level of immunogenicity is obtained, the immunized animal can be bled and the serum isolated and stored, and/or the animal can be used to generate mAbs. mAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified polypeptide, peptide or domain, be it a wild-type or mutant composition. The immunizing composition is administered in a manner effective to stimulate antibody producing cells.

mAbs may be further purified, if desired, using filtration, centrifugation and various chromatographic methods such as HPLC or affinity chromatography. Fragments of the monoclonal antibodies of the invention can be obtained from the monoclonal antibodies so produced by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It also is contemplated that a molecular cloning approach may be used to generate mAbs. For this, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 10⁴ times as many antibodies can be produced and screened in a single round, and that new specificities are generated by H and L chain combination which further increases the chance of finding appropriate antibodies.

Humanized monoclonal antibodies are antibodies of animal origin that have been modified using genetic engineering techniques to replace constant region and/or variable region framework sequences with human sequences, while retaining the original antigen specificity. Such antibodies are commonly derived from rodent antibodies with specificity against human antigens. Such antibodies are generally useful for in vivo therapeutic applications. This strategy reduces the host response to the foreign antibody and allows selection of the human effector functions.

“Humanized” antibodies are also contemplated, as are chimeric antibodies from mouse, rat, or other species, bearing human constant and/or variable region domains, bispecific antibodies, recombinant and engineered antibodies and fragments thereof. The techniques for producing humanized immunoglobulins are well known to those of skill in the art. For example U.S. Pat. No. 5,693,762 discloses methods for producing, and compositions of, humanized immunoglobulins having one or more complementarity determining regions (CDR's). When combined into an intact antibody, the humanized immunoglobulins are substantially non-immunogenic in humans and retain substantially the same affinity as the donor immunoglobulin to the antigen, such as a protein or other compound containing an epitope. Examples of other teachings in this area include U.S. Pat Nos. 6,054,297; 5,861,155; and 6,020,192, all specifically incorporated by reference. Methods for the development of antibodies that are “custom-tailored” to the patient's disease are likewise known and such custom-tailored antibodies are also contemplated.

In yet another embodiment, a single-chain antibody may be employed as an apoptosis modulator. Methods for the production of single-chain antibodies are well known to those of skill in the art. The skilled artisan is referred to U.S. Pat. No. 5,359,046, (incorporated herein by reference) for such methods. A single chain antibody is created by fusing together the variable domains of the heavy and light chains using a short peptide linker, thereby reconstituting an antigen binding site on a single molecule.

Single-chain antibody variable fragments (scFvs) in which the C-terminus of one variable domain is tethered to the N-terminus of the other via a 15 to 25 amino acid peptide or linker, have been developed without significantly disrupting antigen binding or specificity of the binding (Bedzyk et al., 1990; Chaudhary et al., 1990). These Fvs lack the constant regions (Fc) present in the heavy and light chains of the native antibody. Immunotoxins employing single-chain antibodies are described in U.S. Pat. No. 6,099,842, specifically incorporated by reference.

Bispecific antibodies are monoclonal, preferably human or humanized, antibodies that have binding specificities for at least two different antigens. In the present case, one of the binding specificities is for a chimeric polypeptide, the other one is for any other antigen, and preferably for another receptor or receptor subunit. For example, bispecific antibodies specifically binding a chimeric polypeptide and neurotrophic factor, or two different chimeric polypeptides are within the scope of the present invention.

Traditionally, the recombinant production of bispecific antibodies is based on the coexpression of two immunoglobulin heavy chain-light chain pairs, where the two heavy chains have different specificities (Millstein and Cuello (1983)). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, is rather cumbersome, and the product yields are low. Similar procedures are disclosed in WO 93/08829, and in Traunecker et al. (1991).

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH₂ and CH₃ regions. It is preferred to have the first heavy chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are cotransfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance. In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in copending application U.S. Ser. No. 07/931,811, filed Aug. 17, 1992. For further details of generating bispecific antibodies see, for example, Suresh et al. (1986).

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. 4,676,980), and for treatment of HIV infection (WO 91/00360; WO 92/200373; EP 03089). Heteroconjugate antibodies may be made using any convenient cross-linking methods. Suitable cross-linking agents are well known in the art, and are disclosed in U.S. Pat. 4,676,980, along with a number of cross-linking techniques.

A large number of assays involving antibodies are well known to those of skill in the art, including immunodetection such as immunohistochemistry, ELISA, Western blotting, FACS analysis, radioimmunoassay (RIA), immunoradiometric assay, fluoroimmunoassay, chemiluminescent assay, and bioluminescent assay. The steps of various useful immunodetection methods have been described in the scientific literature, such as, e.g., Doolittle M H and Ben-Zeev O, 1999; Gulbis B and Galand P, 1993; De Jager R et al., 1993; and Nakamura et al., 1987, each incorporated herein by reference.

B. Isolating Proteinaceous Compounds

U_{S}3, BAD, or ICP4 may be obtained according to various standard methodologies that are known to those of skill in the art. For example, antibodies specific for U_{S}3 or ICP4 may be used in immunoaffinity protocols to isolate the respective polypeptide from infected cells, in particular, from infected cell lysates. Antibodies are advantageously bound to supports, such as columns or beads, and the immobilized antibodies can be used to pull the U_{S}3 or IPC4 target out of the cell lysate.

Alternatively, expression vectors, rather than viral infections, may be used to generate the polypeptide of interest. A wide variety of expression vectors may be used, including viral vectors. The structure and use of these vectors is discussed further, below. Such vectors may significantly increase the amount of U_{S}3, BAD, and/or ICP4 protein in the cells, and may permit less selective purification methods such as size fractionation (chromatography, centrifugation), ion exchange or affinity chromatograph, and even gel purification. Alternatively, the expression vector may be provided directly to target cells, again as discussed further, below.

U_{S}3, according to the present invention, may advantageously be cleaved into fragments for use in further structural or functional analysis, or in the generation of reagents such as U_{S}3-related polypeptides and U_{S}3-specific antibodies. This can be accomplished by treating purified or unpurified U_{S}3 with a peptidase such as endoproteinase glu-C (Boehringer, Indianapolis, Ind.). Treatment with CNBr is another method by which U_{S}3 fragments may be produced from natural U_{S}3. Recombinant techniques also can be used to produce specific fragments of U_{S}3. It may be that the phosphorylating and apoptosis-inhibiting functions of U_{S}3 reside in distinct domains of the protein. If such is the case, the ability to make domain-specific reagents now has significance. For example, the ability to provide an apoptosis-inhibiting U_{S}3 fragment that does not phosphorylate viral genes may prove to be effective in extending the life of neurons expressing compensatory or therapeutic genes from a viral vector.

Likewise ICP4 or BAD may advantageously be cleaved into fragments for use in further structural or functional analysis, or in the generation of reagents such as ICP4- or BAD-related polypeptides and ICP4- or BAD-specific antibodies. Because the transactivating and apoptosis-inhibiting functions of ICP4 appear to reside in distinct domains, the ability to make domain-specific reagents now has significance. For example, the ability to provide an apoptosis-inhibiting ICP4 fragment that does not transactivate viral genes may prove to be effective in extending the life of neurons expressing compensatory or therapeutic genes from a viral vector. Similarly, BAD has been identified as possessing a “death domain,” which is a region of the polypeptide necessary for BAD prop-apoptotic activity. This region may be separated from the remainder of the BAD polypeptide and used as a modulator of apoptosis or as an agent in a screening method to identify BAD modulators.

It is expected that changes may be made in the sequence of U_{S}3, BAD, or ICP4 while retaining a molecule having the structure and function of the natural U_{S}3, BAD, or ICP4, respectively. For example, certain amino acids may be substituted for other amino acids in a protein structure without appreciable loss of interactive capacity with structures such as, for example, substrate-binding regions. These changes are termed “conservative” in the sense that they preserve the structural and, presumably, required functional qualities of the starting molecule. The importance of U_{S}3 variants is highlighted by the observation, discussed in the examples, that mutants lacking the α4 gene also have an additional mutation in the U_{S}3 gene making it non-functional in apoptosis. The mutation in U_{S}3 was silent and would not have affected the studies carried out by DeLuca et al. (1985) since viral gene expression, including that of U_{S}3, requires a functional α4 gene and apoptosis is a very late event. Even if a rescue had been done, it would not have detected the mutation in U_{S}3 unless the infected cell were probed for the U_{S}3 function.

C. Variants

The importance of ICP4 variants is highlighted by the observation that temperature sensitive (ts) mutants of ICP4 exist that are impaired in their ability to transactivate viral genes at elevated temperatures (above about 39° C.), but retain the apoptosis inhibiting function associated with this polypeptide. Further exploration of this dichotomy should reveal significant information on the regions in which these functions lie. It has been shown that the transactivation domain of ICP4 lies between about residues 100 and 200, the DNA-binding domains lies between about residues 300 and 500, the nuclear localization domain lies between about residues 700 and 750 and the transactivation domain lies between about residues 750 and 1298.

Conservative amino acid substitutions generally are based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as equivalent.

In making such changes, the hydropathic index of amino acids also may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte & Doolittle, 1982). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within ±1 are particularly preferred, and those within ±0.5 are even more particularly preferred.

It also is understood in the art that the substitution of like amino acids can be made effectively on the basis of hydrophilicity, particularly where the polypeptide created is intended for use in immunological embodiments, as in the present case. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e., with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 is preferred, those which are within ±1 are particularly preferred, and those within ±0.5 are even more particularly preferred.

Numerous scientific publications have been devoted to the prediction of secondary structure, and to the identification of epitopes, from analyses of amino acid sequences (Chou & Fasman, 1974a,b; 1978a,b; 1979). Any of these may be used, if desired, to supplement the teachings of Hopp in U.S. Pat. No. 4,554,101. Moreover, computer programs are currently available to assist with predicting antigenic portions and epitopic core regions of proteins. Examples include those programs based upon the Jameson-Wolf analysis (Jameson & Wolf, 1988; Wolf et al., 1988), the program PepPlot® (Brutlag et al., 1990; Weinberger et al., 1985), and other new programs for protein tertiary structure prediction (Fetrow & Bryant, 1993).

Two designations for amino acids are used interchangeably throughout this application, as is common practice in the art. Alanine=Ala (A); Arginine=Arg (R); Aspartate=Asp (D); Asparagine=Asn (N); Cysteine=Cys (C); Glutamate=Glu (E); Glutamine=Gln (Q); Glycine=Gly (G); Histidine=His (H); Isoleucine=Ile (I); Leucine=Leu (L); Lysine=Lys (K); Methionine=Met (M); Phenylalanine=Phe (F); Proline=Pro (P); Serine=Ser (S); Threonine=Thr (T); Tryptophan=Trp (W); Tyrosine=Tyr (Y); Valine=Val (V).

D. Peptides

The present invention encompasses modulators of apoptosis, including proteinaceous compounds that inhibit, induce, or promote apoptosis in a cell, including peptides, as well as fusion proteins, for use in various embodiments of the present invention. The peptides of the invention can be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Peptides with at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 50, 55, 60, 65, 70, 75, 80, 85, 90, 95 or up to about 100 amino acid residues are contemplated by the present invention. Peptides comprising 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 50, 55, 60, 65, 70, 75, 80, 85, 90, 95, 96, 97, 98, 99 and 100 contiguous amino acids of SEQ ID NO: 2, SEQ ID NO: 4, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, and SEQ ID NO: 25 are specifically contemplated as part of the invention.

The compositions of the invention may include a peptide comprising an apoptosis modulator that has been modified to enhance its activity or to render it biologically protected. Biologically protected peptides have certain advantages over unprotected peptides when administered to human subjects and, as disclosed in U.S. Pat. No. 5,028,592, incorporated herein by reference, protected peptides often exhibit increased pharmacological activity.

Compositions for use in the present invention may also comprise peptides that include all L-amino acids, all D-amino acids, or a mixture thereof. The use of D-amino acids may confer additional resistance to proteases naturally found within the human body and are less immunogenic and can therefore be expected to have longer biological half lives.

In addition to the peptidyl compounds described herein, the inventors also contemplate that other sterically similar compounds may be formulated to mimic the key portions of the peptide structure, called peptidomimetics. Mimetics are peptide-containing molecules which mimic elements of protein secondary structure. See, for example, Johnson et al. (1993). The underlying rationale behind the use of peptide mimetics is that the peptide backbone of proteins exists chiefly to orient amino acid side chains in such a way as to facilitate molecular interactions, such as those of receptor and ligand.

Successful applications of the peptide mimetic concept have thus far focused on mimetics of β-turns within proteins. Likely β-turn structures within U_{S}3, BAD, and ICP4 can be predicted by computer-based algorithms as discussed above. Once the component amino acids of the turn are determined, mimetics can be constructed to achieve a similar spatial orientation of the essential elements of the amino acid side chains, as discussed in Johnson et al., supra.

### III. NUCLEIC ACID COMPOSITIONS

Also contemplated by the present invention are nucleic acids encoding U_{S}3, BAD, and ICP4, and fragments thereof. The cDNA sequence for human BAD is provided as SEQ ID NO: 1, while the murine sequence is provided as SEQ ID NO: 3. The gene for HSV-1 U_{S}3 is given in SEQ ID NO: 5. The gene for ICP4 is known as α4. The full length genomic sequence of HSV is known and can be found in Genbank (Accession No. x14112) and is specifically incorporated by reference herein; ICP4 is encoded by identical diploid genes inverted relative to each other, their coding sequences are located from nucleotide 131, 128 to 127, 232 and 147, 104 to 151,000. The U_{S}3 protein coding sequence is from nucleotide 135,222 to 136,667.

Certain embodiments of the present invention involve the synthesis and/or mutation of at least one isolated nucleic acid molecule, such as recombinant expression vectors encoding all or part of the amino acid sequences, such as those shown in SEQ ID NOS: 2, 4, 6, 7, 8, 9, 10, 11, 12, 13, 14, 15, 16, 17, 18, 19, 20, 21, and 22, or one or more modulators of apoptosis. Embodiments of the invention also involve the creation and use of recombinant host cells through the application of DNA recombinant technology, that express one or more U_{S}3 and/or BAD peptides or polypeptides. In certain aspects, a nucleic acid encoding a U_{S}3 and/or BAD peptides or polypeptides or a modulator of apoptosis comprises a wild-type or a mutant nucleic acid. The nucleic acid compositions can, for example, be used in an assay for modulators of apoptosis or for modulators of BAD activity.

Because of the degeneracy of the genetic code, many other nucleic acids also may encode a given U_{S}3, BAD, or a given ICP4. For example, four different three-base codons encode the amino acids alanine, glycine, proline, threonine and valine, while six different codons encode arginine, leucine and serine. Only methionine and tryptophan are encoded by a single codon. A table of amino acids and the corresponding codons is presented herein for use in such embodiments.

In order to generate any nucleic acid encoding U_{S}3, BAD, or ICP4, one need only refer to the preceding codon table. Substitution of the natural codon with any codon encoding the same amino acid will result in a distinct nucleic acid that encodes U_{S}3, BAD, or ICP4 or a variant thereof. As a practical matter, this can be accomplished by site-directed mutagenesis of an existing U_{S}3, BAD, or α4 gene or de novo chemical synthesis of one or more nucleic acids.

TABLE 2

| Amino Acids | | | Codons | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

The preceding observations regarding codon selection, site-directed mutagenesis and chemical synthesis apply with equal force to the discussion of substitutional mutants in the section of peptides. Normally, substitutional mutants are generated by site-directed changes in the nucleic acid designed to alter one or more codons of the coding sequence.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length.

These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule. As used herein, a single stranded nucleic acid may be denoted by the prefix “ss,” a double stranded nucleic acid by the prefix “ds,” and a triple stranded nucleic acid by the prefix “ts.”

A. Nucleobases

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, carboxyalkyl, amino, hydroxyl, halogen (i.e., fluoro, chloro, bromo, or iodo), thiol or alkylthiol moiety. Preferred alkyl (e.g., alkyl, caboxyalkyl, etc.) moieties comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. A table non-limiting, purine and pyrimidine derivatives and analogs is also provided herein below.

A nucleobase may be comprised in a nucleoside or nucleotide, using any chemical or natural synthesis method described herein or known to one of ordinary skill in the art.

TABLE 3
Purine and Pyrmidine Derivatives or Analogs

| Abbr. | Modified base description |
|---|---|
| ac4c | 4-acetylcytidine |
| Chm5u | 5-(carboxyhydroxylmethyl) uridine |
| Cm | 2′-O-methylcytidine |
| Cmnm5s2u | 5-carboxymethylamino-methyl-2-thioridine |
| Cmnm5u | 5-carboxymethylaminomethyluridine |
| D | Dihydrouridine |
| Fm | 2′-O-methylpseudouridine |
| Gal q | Beta,D-galactosylqueosine |
| Gm | 2′-O-methylguanosine |
| I | Inosine |
| I6a | N6-isopentenyladenosine |
| m1a | 1-methyladenosine |
| m1f | 1-methylpseudouridine |
| m1g | 1-methylguanosine |
| m1I | 1-methylinosine |
| m22g | 2,2-dimethylguanosine |
| m2a | 2-methyladenosine |
| m2g | 2-methylguanosine |
| m3c | 3-methylcytidine |
| m5c | 5-methylcytidine |
| m6a | N6-methyladenosine |
| m7g | 7-methylguanosine |
| Mam5u | 5-methylaminomethyluridine |
| Mam5s2u | 5-methoxyaminomethyl-2-thiouridine |
| Man q | Beta,D-mannosylqueosine |
| Mcm5s2u | 5-methoxycarbonylmethyl-2-thiouridine |
| Mcm5u | 5-methoxycarbonylmethyluridine |
| Mo5u | 5-methoxyuridine |
| Ms2i6a | 2-methylthio-N6-isopentenyladenosine |
| Ms2t6a | N-((9-beta-D-ribofuranosyl-2-methylthiopurine-6- |
| | yl)carbamoyl)threonine |
| Mt6a | N-((9-beta-D-ribofuranosylpurine-6-yl)N-methyl- |
| | carbamoyl)threonine |
| Mv | Uridine-5-oxyacetic acid methylester |
| o5u | Uridine-5-oxyacetic acid (v) |
| Osyw | Wybutoxosine |
| P | Pseudouridine |
| Q | Queosine |
| s2c | 2-thiocytidine |
| s2t | 5-methyl-2-thiouridine |
| s2u | 2-thiouridine |
| s4u | 4-thiouridine |
| T | 5-methyluridine |
| t6a | N-((9-beta-D-ribofuranosylpurine-6-yl)carbamoyl)threonine |
| Tm | 2′-O-methyl-5-methyluridine |
| Um | 2′-O-methyluridine |
| Yw | Wybutosine |
| X | 3-(3-amino-3-carboxypropyl)uridine, (acp3)u |

B. Nucleosides

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the 1′-position of a 5-carbon sugar. In another non-limiting example, a nucleoside comprising a pyrimidine nucleobase (i.e., C, T or U) typically covalently attaches a 1 position of a pyrimidine to a 1′-position of a 5-carbon sugar (Komberg and Baker, 1992).

C. Nucleotides

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

D. Nucleic Acid Analogs

A nucleic acid may comprise, or be composed entirely of, a derivative or analog of a nucleobase, a nucleobase linker moiety and/or backbone moiety that may be present in a naturally occurring nucleic acid. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference).

Additional non-limiting examples of nucleosides, nucleotides or nucleic acids comprising 5-carbon sugar and/or backbone moiety derivatives or analogs, include those in U.S. Pat. No. 5,681,947 which describes oligonucleotides comprising purine derivatives that form triple helixes with and/or prevent expression of dsDNA; U.S. Pat. No. 5,652,099 and 5,763,167 which describe nucleic acids incorporating fluorescent analogs of nucleosides found in DNA or RNA, particularly for use as fluorescent nucleic acids probes; U.S. Pat. No. 5,614,617 which describes oligonucleotide analogs with substitutions on pyrimidine rings that possess enhanced nuclease stability; U.S. Pat. Nos. 5,670,663, 5,872,232 and 5,859,221 which describe oligonucleotide analogs with modified 5-carbon sugars (i.e., modified 2′-deoxyfuranosyl moieties) used in nucleic acid detection; U.S. Pat. No. 5,446,137 which describes oligonucleotides comprising at least one 5-carbon sugar moiety substituted at the 4′ position with a substituent other than hydrogen that can be used in hybridization assays; U.S. Pat. No. 5,886,165 which describes oligonucleotides with both deoxyribonucleotides with 3′-5′ internucleotide linkages and ribonucleotides with 2′-5′ internucleotide linkages; U.S. Pat. No. 5,714,606 which describes a modified internucleotide linkage wherein a 3′-position oxygen of the internucleotide linkage is replaced by a carbon to enhance the nuclease resistance of nucleic acids; U.S. Pat. No. 5,672,697 which describes oligonucleotides containing one or more 5′ methylene phosphonate internucleotide linkages that enhance nuclease resistance; U.S. Pat. Nos. 5,466,786 and 5,792,847 which describe the linkage of a substituent moiety which may comprise a drug or label to the 2′ carbon of an oligonucleotide to provide enhanced nuclease stability and ability to deliver drugs or detection moieties; U.S. Pat. No. 5,223,618 which describes oligonucleotide analogs with a 2 or 3 carbon backbone linkage attaching the 4′ position and 3′ position of adjacent 5-carbon sugar moiety to enhanced cellular uptake, resistance to nucleases and hybridization to target RNA; U.S. Pat. No. 5,470,967 which describes oligonucleotides comprising at least one sulfamate or sulfamide internucleotide linkage that are useful as nucleic acid hybridization probe; U.S. Pat. Nos. 5,378,825, 5,777,092, 5,623,070, 5,610,289 and 5,602,240 which describe oligonucleotides with three or four atom linker moiety replacing phosphodiester backbone moiety used for improved nuclease resistance, cellular uptake and regulating RNA expression; U.S. Pat. No. 5,858,988 which describes hydrophobic carrier agent attached to the 2′-O position of oligonucleotides to enhanced their membrane permeability and stability; U.S. Pat. No. 5,214,136 which describes oligonucleotides conjugated to anthraquinone at the 5′ terminus that possess enhanced hybridization to DNA or RNA; enhanced stability to nucleases; U.S. Pat. No. 5,700,922 which describes PNA-DNA-PNA chimeras wherein the DNA comprises 2′-deoxy-erythro-pentofuranosyl nucleotides for enhanced nuclease resistance, binding affinity, and ability to activate RNase H; and U.S. Pat. No. 5,708,154 which describes RNA linked to a DNA to form a DNA-RNA hybrid.

In a non-limiting example, one or more nucleic acid analogs may be prepared containing about 3, about 5, about 8, about 10 to about 14, or about 15, about 20, about 30, about 40, about 50, about 100, about 200, about 500, about 1,000, about 2,000, about 3,000, about 5,000, about 10,000, about 15,000, about 20,000, about 30,000, about 50,000, about 100,000, about 250,000, about 500,000, about 750,000, to about 1,000,000 nucleotides in length, as well as constructs of greater size, up to and including chromosomal sizes (including all intermediate lengths and intermediate ranges). Such analogs may be implemented with respect to SEQ ID NOS:1, 3, or 5, as provided herein.

E. Polyether and Peptide Nucleic Acids

In certain embodiments, it is contemplated that a nucleic acid comprising a derivative or analog of a nucleoside or nucleotide may be used in the methods and compositions of the invention. A non-limiting example is a “polyether nucleic acid”, described in U.S. Pat. No. 5,908,845, incorporated herein by reference. In a polyether nucleic acid, one or more nucleobases are linked to chiral carbon atoms in a polyether backbone.

Another non-limiting example is a “peptide nucleic acid”, also known as a “PNA”, “peptide-based nucleic acid analog” or “PENAM”, described in U.S. Pat. Nos. 5,786,461, 5891,625, 5,773,571, 5,766,855, 5,736,336, 5,719,262, 5,714,331, 5,539,082, and WO 92/20702, each of which is incorporated herein by reference. Peptide nucleic acids generally have enhanced sequence specificity, binding properties, and resistance to enzymatic degradation in comparison to molecules such as DNA and RNA (Egholm et al., 1993; PCT/EP/01219). A peptide nucleic acid generally comprises one or more nucleotides or nucleosides that comprise a nucleobase moiety, a nucleobase linker moiety that is not a 5-carbon sugar, and/or a backbone moiety that is not a phosphate backbone moiety. Examples of nucleobase linker moieties described for PNAs include aza nitrogen atoms, amido and/or ureido tethers (see for example, U.S. Pat. No. 5,539,082). Examples of backbone moieties described for PNAs include an aminoethylglycine, polyamide, polyethyl, polythioamide, polysulfinamide or polysulfonamide backbone moiety.

In certain embodiments, a nucleic acid analogue such as a peptide nucleic acid may be used to inhibit nucleic acid amplification, such as in PCR, to reduce false positives and discriminate between single base mutants, as described in U.S. Pat. No. 5,891,625. Other modifications and uses of nucleic acid analogs are known in the art, and are encompassed by the nucleic acid encoding for apoptosis modulators. In a non-limiting example, U.S. Pat. No. 5,786,461 describes PNAs with amino acid side chains attached to the PNA backbone to enhance solubility of the molecule. In another example, the cellular uptake property of PNAs is increased by attachment of a lipophilic group. U.S. application Ser. No. 117,363 describes several alkylamino moieties used to enhance cellular uptake of a PNA. Another example is described in U.S. Pat. Nos. 5,766,855, 5,719,262, 5,714,331 and 5,736,336, which describe PNAs comprising naturally and non-naturally occurring nucleobases and alkylamine side chains that provide improvements in sequence specificity, solubility and/or binding affinity relative to a naturally occurring nucleic acid.

F. Antisense and Ribozymes

The invention also concerns modulators of apoptosis including molecules that directly affect BAD or U_{S}3 transcripts encoding promoters and inhibitors, respectively, of apoptosis. Antisense and ribozyme molecules target a particular sequence to achieve a reduction or elimination of a particular polypeptide, such as apoptosis modulators. Thus, it is contemplated that nucleic acid molecules that are identical or complementary to all or part of SEQ ID NO: 1, 3, and 5 are included as part of the invention. For example, an antisense molecule complementary to the transcript of a BAD-encoding nucleic acid may be used in the context of the present invention to inhibit apoptosis. Similarly, an antisense molecule complementary to the transcript of a U_{S}3-encoding nucleic acid may be used in the context of the present invention to relieve apoptosis inhibition and promote apoptosis of a cell.

a. Antisense Molecules

Antisense methodology takes advantage of the fact that nucleic acids tend to pair with “complementary” sequences. By complementary, it is meant that polynucleotides are those which are capable of base-pairing according to the standard Watson-Crick complementarily rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNAs, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs may include regions complementary to intron/exon splice junctions. Thus, antisense constructs with complementarily to regions within 50-200 bases of an intron-exon splice junction may be used. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

b. Ribozymes

The use of apoptosis modulator-specific ribozymes, is claimed in the present application. The following information is provided in order to compliment the earlier section and to assist those of skill in the art in this endeavor.

Ribozymes are RNA-protein complexes that cleave nucleic acids in a site-specific fashion. Ribozymes have specific catalytic domains that possess endonuclease activity (Kim and Cech, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cech et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence specific cleavage/ligation reactions involving nucleic acids (Joyce, 1989; Cech et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990; Sioud et al., 1992). Recently, it was reported that ribozymes elicited genetic changes in some cell lines to which they were applied; the altered genes included the oncogenes H-ras, c-fos and genes of HIV. Most of this work involved the modification of a target mRNA, based on a specific mutant codon that is cleaved by a specific ribozyme. In light of the information included herein and the knowledge of one of ordinary skill in the art, the preparation and use of additional ribozymes that are specifically targeted to a given gene will now be straightforward.

Several different ribozyme motifs have been described with RNA cleavage activity (reviewed in Symons, 1992). Examples that would be expected to function equivalently for the down regulation of apotosis modulators include sequences from the Group I self splicing introns including tobacco ringspot virus (Prody et al., 1986), avocado sunblotch viroid (Palukaitis et al., 1979; Symons, 1981), and Lucerne transient streak virus (Forster and Symons, 1987). Sequences from these and related viruses are referred to as hammerhead ribozymes based on a predicted folded secondary structure.

Other suitable ribozymes include sequences from RNase P with RNA cleavage activity (Yuan et al., 1992; Yuan and Altman, 1994), hairpin ribozyme structures (Berzal-Herranz et al., 1992; Chowrira et al., 1993) and hepatitis 6 virus based ribozymes (Perrotta and Been, 1992). The general design and optimization of ribozyme directed RNA cleavage activity has been discussed in detail (Haseloff and Gerlach, 1988, Symons, 1992; Chowrira, et al., 1994; and Thompson, et al., 1995).

The other variable on ribozyme design is the selection of a cleavage site on a given target RNA. Ribozymes are targeted to a given sequence by virtue of annealing to a site by complimentary base pair interactions. Two stretches of homology are required for this targeting. These stretches of homologous sequences flank the catalytic ribozyme structure defined above. Each stretch of homologous sequence can vary in length from 7 to 15 nucleotides. The only requirement for defining the homologous sequences is that, on the target RNA, they are separated by a specific sequence which is the cleavage site. For hammerhead ribozymes, the cleavage site is a dinucleotide sequence on the target RNA, uracil (U) followed by either an adenine, cytosine or uracil (A,C or U; Perriman, et al., 1992; Thompson, et al., 1995). The frequency of this dinucleotide occurring in any given RNA is statistically 3 out of 16. Therefore, for a given target messenger RNA of 1000 bases, 187 dinucleotide cleavage sites are statistically possible.

Designing and testing ribozymes for efficient cleavage of a target RNA is a process well known to those skilled in the art. Examples of scientific methods for designing and testing ribozymes are described by Chowrira et al. (1994) and Lieber and Strauss (1995), each incorporated by reference. The identification of operative and preferred sequences for use in apoptosis modulator-targeted ribozymes is simply a matter of preparing and testing a given sequence, and is a routinely practiced “screening” method known to those of skill in the art.

G. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 1989 and 2001, incorporated herein by reference).

H. Purification of Nucleic Acids

A nucleic acid may be purified on polyacrylamide gels, cesium chloride centrifugation gradients, or by any other means known to one of ordinary skill in the art (see for example, Sambrook et al., 1989, incorporated herein by reference).

In certain aspect, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

I. Nucleic Acid Segments

In certain embodiments, the nucleic acid is a nucleic acid segment. As used herein, the term “nucleic acid segment,” are fragments of a nucleic acid, such as for non-limiting example, those that encode only part of a U_{S}3 or BAD peptide or polypeptide sequence. Thus, a “nucleic acid segment” may comprise any part of a gene sequence, of from about 10 nucleotides to the full length of the U_{S}3 or BAD peptide- or polypeptide-encoding region.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created:

n to n+y

where n is an integer from 1 to the last number of the sequence and y is the length of the nucleic acid segment minus one, where n+y does not exceed the last number of the sequence. Thus, for a 10-mer, the nucleic acid segments correspond to bases 1 to 10, 2 to 11, 3 to 12 . . . and so on. For a 15-mer, the nucleic acid segments correspond to bases 1 to 15, 2 to 16, 3 to 17 . . . and so on. For a 20-mer, the nucleic segments correspond to bases 1 to 20, 2 to 21, 3 to 22 . . . and so on. In certain embodiments, the nucleic acid segment may be a probe or primer. As used herein, a “probe” generally refers to a nucleic acid used in a detection method or composition. As used herein, a “primer” generally refers to a nucleic acid used in an extension or amplification method or composition.

In a non-limiting example, nucleic acid segments may contain up to 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 4000, or 5000 nucleotides. Contiguous nucleic acids segments of SEQ ID NO: 1, 3, or 5 may be used in the present invention. Nucleic acid segments may also contain up to 10,000, 20,000, 30,000, 50,000, 100,000, 250,000, 500,000, 750,000, to 1,000,000 nucleotides in length, as well as constructs of greater size, up to and including chromosomal sizes are contemplated for use in the present invention. Furthermore, nucleic acids, including expression constructs, may comprise at least 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 4000, or 5000 contiguous nucleic acid residues or nucleotides from SEQ ID NO: 1, 3, or 5.

J. Nucleic Acid Complements

The present invention also encompasses a nucleic acid that is complementary to the nucleic acid encoding for BAD or a herpesvirus U_{S}3 polypeptide. In particular embodiments the invention encompasses a nucleic acid or a nucleic acid segment complementary to the sequence set forth in SEQ ID NO: 1, 3, or 5. A nucleic acid is “complement(s)” or is “complementary” to another nucleic acid when it is capable of base-pairing with another nucleic acid according to the standard Watson-Crick, Hoogsteen or reverse Hoogsteen binding complementarity rules. As used herein “another nucleic acid” may refer to a separate molecule or a spatial separated sequence of the same molecule.

As used herein, the term “complementary” or “complement(s)” also refers to a nucleic acid comprising a sequence of consecutive nucleobases or semiconsecutive nucleobases (e.g., one or more nucleobase moieties are not present in the molecule) capable of hybridizing to another nucleic acid strand or duplex even if less than all the nucleobases do not base pair with a counterpart nucleobase. In certain embodiments, a “complementary” nucleic acid comprises a sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range derivable therein, of the nucleobase sequence is capable of base-pairing with a single or double stranded nucleic acid molecule during hybridization. In certain embodiments, the term “complementary” refers to a nucleic acid that may hybridize to another nucleic acid strand or duplex in stringent conditions, as would be understood by one of ordinary skill in the art.

In certain embodiments, a “partly complementary” nucleic acid comprises a sequence that may hybridize in low stringency conditions to a single or double stranded nucleic acid, or contains a sequence in which less than about 70% of the nucleobase sequence is capable of base-pairing with a single or double stranded nucleic acid molecule during hybridization.

K. Hybridization

As used herein, “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

As used herein “wild-type” refers to the naturally occurring sequence of a nucleic acid at a genetic locus in the genome of an organism, or a sequence transcribed or translated from such a nucleic acid. Thus, the term “wild-type” also may refer to an amino acid sequence encoded by a nucleic acid. As a genetic locus may have more than one sequence or alleles in a population of individuals, the term “wild-type” encompasses all such naturally occurring allele(s). As used herein the term “polymorphic” means that variation exists (i.e., two or more alleles exist) at a genetic locus in the individuals of a population. As used herein “mutant” refers to a change in the sequence of a nucleic acid or its encoded protein, polypeptide or peptide that is the result of the hand of man.

The present invention also concerns the isolation or creation of a recombinant construct or a recombinant host cell through the application of recombinant nucleic acid technology known to those of skill in the art or as described herein. A recombinant construct or host cell may express an U_{S}3, BAD, or apoptosis modulator protein, peptide or peptide, or at least one biologically functional equivalent thereof. The recombinant host cell may be a prokaryotic cell. In a more preferred embodiment, the recombinant host cell is a eukaryotic cell. As used herein, the term “engineered” or “recombinant” cell is intended to refer to a cell into which a recombinant gene, such as a gene encoding an U_{S}3, BAD, or apoptosis modulator, has been introduced. Therefore, engineered cells are distinguishable from naturally occurring cells which do not contain a recombinantly introduced gene. Engineered cells are thus cells having a gene or genes introduced through the hand of man. Recombinantly introduced genes will either be in the form of a cDNA gene (i.e., they will not contain introns), a copy of a genomic gene, or will include genes positioned adjacent to a promoter not naturally associated with the particular introduced gene.

In certain embodiments, a “gene” refers to a nucleic acid that is transcribed. In certain aspects, the gene includes regulatory sequences involved in transcription, or message production or composition. In particular embodiments, the gene comprises transcribed sequences that encode for a protein, polypeptide or peptide. As will be understood by those in the art, this function term “gene” includes both genomic sequences, RNA or cDNA sequences or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express using nucleic acid manipulation technology, proteins, polypeptides, domains, peptides, fusion proteins, mutants and/or such like. The term “cDNA” refers to that portion of a gene that is transcribed.

The nucleic acid(s) of the present invention, regardless of the length of the sequence itself, may be combined with other nucleic acid sequences, including but not limited to, promoters, enhancers, polyadenylation signals, restriction enzyme sites, multiple cloning sites, coding segments, and the like, to create one or more nucleic acid construct(s). As used herein, a “nucleic acid construct” is a nucleic acid engineered or altered by the hand of man, and generally comprises one or more nucleic acid sequences organized by the hand of man.

In a non-limiting example, one or more nucleic acid constructs may be prepared containing about 3, about 5, about 8, about 10 to about 14, or about 15, about 20, about 30, about 40, about 50, about 100, about 200, about 500, about 1,000, about 2,000, about 3,000, about 5,000, about 10,000, about 15,000, about 20,000, about 30,000, about 50,000, about 100,000, about 250,000, about 500,000, about 750,000, to about 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges”, as used herein, means any length or range including or between the quoted values (i.e., all integers including and between such values). Non-limiting examples of intermediate lengths include about 11, about 12, about 13, about 16, about 17, about 18, about 19, etc.; about 21, about 22, about 23, etc.; about 31, about 32, etc.; about 51, about 52, about 53, etc.; about 101, about 102, about 103, etc.; about 151, about 152, about 153, etc.; about 1,001, about 1002, etc,; about 50,001, about 50,002, etc; about 750,001, about 750,002, etc.; about 1,000,001, about 1,000,002, etc. Non-limiting examples of intermediate ranges include about 3 to about 32, about 150 to about 500,001, about 3,032 to about 7,145, about 5,000 to about 15,000, about 20,007 to about 1,000,003, etc. Such constructs may be implemented and used with respect to SEQ ID NO: 1, 3,or5.

The term “functionally equivalent codon” is used herein to refer to codons that encode the same amino acid, such as the six codons for arginine and serine, and also refers to codons that encode biologically equivalent amino acids. For optimization of expression of genes in human cells, the codons are shown in Table 4 in preference of use from left to right. Thus, the most preferred codon for alanine is thus “GCC”, and the least is “GCG”. Codon usage for various organisms and organelles can be found at the website http://www.kazusa.or.jp/codon/, incorporated herein by reference, allowing one of skill in the art to optimize codon usage for expression in various organisms using the disclosures herein. Thus, it is contemplated that codon usage may be optimized for other animals, as well as other organisms such as a prokaryote (e.g., an eubacteria, an archaea), an eukaryote (e.g., a protist, a plant, a fungi, an animal), a virus and the like, as well as organelles that contain nucleic acids, such as mitochondria, chloroplasts and the like, based on the preferred codon usage as would be known to those of ordinary skill in the art.

It will also be understood that amino acid sequences or nucleic acid sequences may include additional residues, such as additional N- or C-terminal amino acids or 5′ or 3′ sequences, or various combinations thereof, and yet still be essentially as set forth in one of the sequences disclosed herein, so long as the sequence meets the criteria set forth above, including the maintenance of biological protein, polypeptide or peptide activity where expression of a proteinaceous composition is concerned. The addition of terminal sequences particularly applies to nucleic acid sequences that may, for example, include various non-coding sequences flanking either of the 5′ and/or 3′ portions of the coding region or may include various internal sequences, i.e., introns, which are known to occur within genes.

The nucleic acids of the present invention encompass biologically functional equivalent U_{S}3, BAD, or apoptosis modulator proteins, polypeptides, or peptides. Such sequences may arise as a consequence of codon redundancy or functional equivalency that are known to occur naturally within nucleic acid sequences or the proteins, polypeptides or peptides thus encoded. Alternatively, functionally equivalent proteins, polypeptides or peptides may be created via the application of recombinant DNA technology, in which changes in the protein, polypeptide or peptide structure may be engineered, based on considerations of the properties of the amino acids being exchanged. Changes designed by man may be introduced, for example, through the application of site-directed mutagenesis techniques as discussed herein below, e.g., to introduce improvements or alterations to the antigenicity of the protein, polypeptide or peptide, or to test mutants in order to examine U_{S}3, BAD, or apoptosis modulator protein, polypeptide or peptide activity at the molecular level.

Fusion proteins, polypeptides or peptides may be prepared, e.g., where the coding regions are aligned within the same expression unit with other proteins, polypeptides or peptides having desired functions. Non-limiting examples of such desired functions of expression sequences include purification or immunodetection purposes for the added expression sequences, e.g., proteinaceous compositions that may be purified by affinity chromatography or the enzyme labeling of coding regions, respectively.

As used herein an “organism” may be a prokaryote, eukaryote, virus and the like. As used herein the term “sequence” encompasses both the terms “nucleic acid” and “proteinaceous composition.” As used herein, the term “proteinaceous composition” encompasses the terms “protein”, “polypeptide” and “peptide.” As used herein “artificial sequence” refers to a sequence of a nucleic acid not derived from sequence naturally occurring at a genetic locus, as well as the sequence of any proteins, polypeptides or peptides encoded by such a nucleic acid. A “synthetic sequence”, refers to a nucleic acid or proteinaceous composition produced by chemical synthesis in vitro, rather than enzymatic production in vitro (i.e., an “enzymatically produced” sequence) or biological production in vivo (i.e., a “biologically produced” sequence).

L. Vectors and Expression Constructs

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Sambrook et al., 1988 and Ausubel et al., 1994, both incorporated herein by reference).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operable linked coding sequence in a particular host cell. In addition to control sequences that govern transcription and translation, vectors and expression vectors may contain nucleic acid sequences that serve other functions as well and are described infra.

In order to express an U_{S}3 peptide or polypeptide, or an antisense U_{S}3 transcript, it is necessary to provide an U_{S}3 gene in an expression vehicle. Similarly to express a BAD peptide or polypeptide, or an antisense BAD transcript, it is necessary to provide a BAD cDNA in an expression vehicle. The appropriate nucleic acid can be inserted into an expression vector by standard subcloning techniques. For example, an E. coli or baculovirus expression vector is used to produce recombinant polypeptide in vitro. The manipulation of these vectors is well known in the art. In one embodiment, the protein is expressed as a fusion protein with β-gal, allowing rapid affinity purification of the protein. Examples of such fusion protein expression systems are the glutathione S-transferase system (Pharmacia, Piscataway, N.J.), the maltose binding protein system (NEB, Beverley, Mass.), the FLAG system (IBI, New Haven, Conn.), and the 6×His system (Qiagen, Chatsworth, Calif.).

Some of these fusion systems produce recombinant protein bearing only a small number of additional amino acids, which are unlikely to affect the functional capacity of the recombinant protein. For example, both the FLAG system and the 6×His system add only short sequences, both of which are known to be poorly antigenic and which do not adversely affect folding of the protein to its native conformation. Other fusion systems produce proteins where it is desirable to excise the fusion partner from the desired protein. In another embodiment, the fusion partner is linked to the recombinant protein by a peptide sequence containing a specific recognition sequence for a protease. Examples of suitable sequences are those recognized by the Tobacco Etch Virus protease (Life Technologies, Gaithersburg, Md.) or Factor Xa (New England Biolabs, Beverley, Mass.).

Recombinant bacterial cells, for example E. coli, are grown in any of a number of suitable media, for example LB, and the expression of the recombinant polypeptide induced by adding IPTG to the media or switching incubation to a higher temperature. After culturing the bacteria for a further period of between 2 and 24 hours, the cells are collected by centrifugation and washed to remove residual media. The bacterial cells are then lysed, for example, by disruption in a cell homogenizer and centrifuged to separate the dense inclusion bodies and cell membranes from the soluble cell components. This centrifugation can be performed under conditions whereby the dense inclusion bodies are selectively enriched by incorporation of sugars such as sucrose into the buffer and centrifugation at a selective speed.

If the recombinant protein is expressed in the inclusion bodies, as is the case in many instances, these can be washed in any of several solutions to remove some of the contaminating host proteins, then solubilized in solutions containing high concentrations of urea (e.g. 8M) or chaotropic agents such as guanidine hydrochloride in the presence of reducing agents such as β-mercaptoethanol or DTT (dithiothreitol).

Under some circumstances, it may be advantageous to incubate the polypeptide for several hours under conditions suitable for the protein to undergo a refolding process into a conformation which more closely resembles that of the native protein. Such conditions generally include low protein concentrations less than 500 μg/ml, low levels of reducing agent, concentrations of urea less than 2 M and often the presence of reagents such as a mixture of reduced and oxidized glutathione which facilitate the interchange of disulphide bonds within the protein molecule.

The refolding process can be monitored, for example, by SDS-PAGE or with antibodies which are specific for the native molecule (which can be obtained from animals vaccinated with the native molecule isolated from parasites). Following, refolding, the protein can then be purified further and separated from the refolding mixture by chromatography on any of several supports including ion exchange resins, gel permeation resins or on a variety of affinity columns.

In yet another embodiment, the expression system used is one driven by the baculovirus polyhedron promoter. The gene encoding the protein can be manipulated by standard techniques in order to facilitate cloning into the baculovirus vector. A preferred baculovirus vector is the pBlueBac vector (Invitrogen, Sorrento, Calif.). The vector carrying the gene of interest is transfected into Spodoptera frugiperda (Sf9) cells by standard protocols, and the cells are cultured and processed to produce the recombinant protein. Mammalian cells exposed to baculoviruses become infected and may express the foreign gene only. This way one can transduce all cells and express the gene in dose dependent manner.

There also are a variety of eukaryotic vectors that provide a suitable vehicle in which recombinant polypeptide can be produced. HSV itself has been used in tissue culture to express a large number of exogenous genes as well as for high level expression of its endogenous genes. For example, the chicken ovalbumin gene has been expressed from HSV using an α promoter. Herz and Roizman (1983). The lacZ gene also has been expressed under a variety of HSV promoters.

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. Thus, in certain embodiments, expression includes both transcription of a gene and translation of a RNA into a gene product. In other embodiments, expression only includes transcription of the nucleic acid, for example, to generate antisense constructs.

In preferred embodiments, the nucleic acid is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

The particular promoter that is employed to control the expression of a nucleic acid is not believed to be critical, so long as it is capable of expressing the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter. Preferred promoters include those derived from HSV, including the U_{S}3, or the α4 promoter. Another preferred embodiment is the tetracycline controlled promoter.

In various other embodiments, the human cytomegalovirus (CMV) immediate early gene promoter, the SV40 early promoter and the Rous sarcoma virus long terminal repeat can be used to obtain high-level expression of transgenes. The use of other viral or mammalian cellular or bacterial phage promoters which are well-known in the art to achieve expression of a transgene is contemplated as well, provided that the levels of expression are sufficient for a given purpose. Tables 4 and 5 list several elements/promoters which may be employed, in the context of the present invention, to regulate the expression of a transgene. This list is not exhaustive of all the possible elements involved but, merely, to be exemplary thereof.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Additionally any promoter/enhancer combination (as per the Eukaryotic Promoter Data Base EPDB) could also be used to drive expression of a transgene. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

TABLE 4

PROMOTER
Immunoglobulin Heavy Chain
Immunoglobulin Light Chain
T-Cell Receptor
HLA DQ α and DQ β
β-Interferon
Interleukin-2
Interleukin-2 Receptor
MHC Class II 5
MHC Class II HLA-DRα
β-Actin
Muscle Creatine Kinase
Prealbumin (Transthyretin)
Elastase I
Metallothionein
Collagenase
Albumin Gene
α-Fetoprotein
τ-Globin
β-Globin
c-fos
c-HA-ras
Insulin
Neural Cell Adhesion Molecule (NCAM)
α_{1-Antitrypsin}
H2B (TH2B) Histone
Mouse or Type I Collagen
Glucose-Regulated Proteins (GRP94 and GRP78)
Rat Growth Hormone
Human Serum Amyloid A (SAA)
Troponin I (TN I)
Platelet-Derived Growth Factor
Duchenne Muscular Dystrophy
SV40
Polyoma
Retroviruses
Papilloma Virus
Hepatitis B Virus
Human Immunodeficiency Virus
Cytomegalovirus
Gibbon Ape Leukemia Virus

TABLE 5

| Element | Inducer |
|---|---|
| MT II | Phorbol Ester (TPA) |
| | Heavy metals |
| MMTV (mouse mammary tumor | Glucocorticoids |
| virus) | |
| β-Interferon | poly(rI)X |
| | poly(rc) |
| Adenovirus 5 E2 | Ela |
| c-jun | Phorbol Ester (TPA), H₂O₂ |
| Collagenase | Phorbol Ester (TPA) |
| Stromelysin | Phorbol Ester (TPA), IL-1 |
| SV40 | Phorbol Ester (TPA) |
| Murine MX Gene | Interferon, Newcastle Disease Virus |
| GRP78 Gene | A23187 |
| α-2-Macroglobulin | IL-6 |
| Vimentin | Serum |
| MHC Class I Gene H-2kB | Interferon |
| HSP70 | Ela, SV40 Large T Antigen |
| Proliferin | Phorbol Ester-TPA |
| Tumor Necrosis Factor | FMA |
| Thyroid Stimulating Hormone α | Thyroid Hormone |
| Gene | |

Use of the baculovirus system will involve high level expression from the powerful polyhedron promoter.

One will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal and the bovine growth hormone polyadenylation signal, convenient and known to function well in various target cells. Also contemplated as an element of the expression cassette is a terminator. These elements can serve to enhance message levels and to minimize read through from the cassette into other sequences.

A specific initiation signal also may be required for efficient translation of coding sequences. These signals include the ATG initiation codon and adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements (Bittner et al, 1987).

In various embodiments of the invention, the expression construct may comprise a virus or engineered construct derived from a viral genome. The ability of certain viruses to enter cells via receptor-mediated endocytosis and to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986) and adeno-associated viruses. Retroviruses also are attractive gene transfer vehicles (Nicolas and Rubenstein, 1988; Temin, 1986) as are vaccinia virus (Ridgeway, 1988) and adeno-associated virus (Ridgeway, 1988). Such vectors may be used to (i) transform cell lines in vitro for the purpose of expressing proteins of interest or (ii) to transform cells in vitro or in vivo to provide therapeutic polypeptides in a gene therapy scenario.

In a preferred embodiment, the vector is HSV. Because HSV is neurotropic, it has generated considerable interest in treating nervous system disorders. Moreover, the ability of HSV to establish latent infections in non-dividing neuronal cells without integrating into the host cell chromosome or otherwise altering the host cell's metabolism, along with the existence of a promoter that is active during latency. And though much attention has focused on the neurotropic applications of HSV, this vector also can be exploited for other tissues.

Another factor that makes HSV an attractive vector is the size and organization of the genome. Because HSV is large, incorporation of multiple genes or expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations.

HSV also is relatively easy to manipulate and can be grown to high titers. Thus, delivery is less of a problem, both in terms of volumes needed to attain sufficient MOI and in a lessened need for repeat dosings. For a review of HSV as a gene therapy vector, see Glorioso et al. (1995).

1. Viral Vectors

Viral vectors are a kind of expression construct that utilizes viral sequences to introduce nucleic acid and possibly proteins into a cell. The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Vector components of the present invention may be a viral vector that encode one or more candidate substance or other components such as, for example, an immunomodulator or adjuvant for the candidate substance. Non-limiting examples of virus vectors that may be used to deliver a nucleic acid of the present invention are described below.

a. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992).

b. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno-associated virus (AAV) is an attractive vector system for use in the candidate substances of the present invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

C. Retroviral Vectors

Retroviruses have promise as an antigen delivery vectors in vaccines of the candidate substances due to their ability to integrate their genes into the host genome, transferring a large amount of foreign genetic material, infecting a broad spectrum of species and cell types and of being packaged in special cell-lines (Miller, 1992).

In order to construct a vaccine retroviral vector, a nucleic acid (e.g., one encoding an U_{S}3, BAD, or apoptosis modulator or therapeutic agent antigen of interest) is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

d. Other Viral Vectors

Other viral vectors may be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

e. Delivery using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

2. Vector Delivery and Cell Transformation

Suitable methods for nucleic acid delivery for transformation of an organelle, a cell, a tissue or an organism for use with the current invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by ex vivo transfection (Wilson et al., 1989, Nabel et al., 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. Nos. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al, 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (WO 94/09699 and WO 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al, 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed.

3. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells which do not contain a recombinantly introduced nucleic acid.

In certain embodiments, it is contemplated that RNAs or proteinaceous sequences may be co-expressed with other selected RNAs or proteinaceous sequences in the same host cell. Co-expression may be achieved by co-transfecting the host cell with two or more distinct recombinant vectors. Alternatively, a single recombinant vector may be constructed to include multiple distinct coding regions for RNAs, which could then be expressed in host cells transfected with the single vector.

In certain embodiments, the host cell or tissue may be comprised in at least one organism. In certain embodiments, the organism may be, but is not limited to, a prokaryote (e.g., a eubacteria, an archaea) or an eukaryote, as would be understood by one of ordinary skill in the art (see, for example, webpage http://phylogeny.arizona.edu/tree/phylogeny.html).

Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Cell types available for vector replication and/or expression include, but are not limited to, bacteria, such as E. coli (e.g., E. coli strain RR1, E. coli LE392, E. coli B, E. coli X 1776 (ATCC No. 31537) as well as E. coli W3110 (F-, lambda-, prototrophic, ATCC No. 273325), DH5α, JM109, and KC8, bacilli such as Bacillus subtilis; and other enterobacteriaceae such as Salmonella typhimurium, Serratia marcescens, various Pseudomonas specie, as well as a number of commercially available bacterial hosts such as SURE® Competent Cells and SOLOPACK™ Gold Cells (STRATAGENE®, La Jolla). In certain embodiments, bacterial cells such as E. coli LE392 are particularly contemplated as host cells for phage viruses.

Examples of eukaryotic host cells for replication and/or expression of a vector include, but are not limited to, HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

It is an aspect of the present invention that the nucleic acid compositions described herein may be used in conjunction with a host cell. For example, a host cell may be transfected using all or part of SEQ ID NO: 1 or any of SEQ ID NOS; 3 or 5.

4. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

Other examples of expression systems include STRATAGENE®'S COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-Rex™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

It is contemplated that the proteins, polypeptides or peptides produced by the methods of the invention may be “overexpressed,” i.e., expressed in increased levels relative to its natural expression in cells. Such overexpression may be assessed by a variety of methods, including radio-labeling and/or protein purification. However, simple and direct methods are preferred, for example, those involving SDS/PAGE and protein staining or western blotting, followed by quantitative analyses, such as densitometric scanning of the resultant gel or blot. A specific increase in the level of the recombinant protein, polypeptide or peptide in comparison to the level in natural cells is indicative of overexpression, as is a relative abundance of the specific protein, polypeptides or peptides in relation to the other proteins produced by the host cell, e.g., visible on a gel.

In some embodiments, the expressed proteinaceous sequence forms an inclusion body in the host cell, the host cells are lysed, for example, by disruption in a cell homogenizer, washed and/or centrifuged to separate the dense inclusion bodies and cell membranes from the soluble cell components. This centrifugation can be performed under conditions whereby the dense inclusion bodies are selectively enriched by incorporation of sugars, such as sucrose, into the buffer and centrifugation at a selective speed. Inclusion bodies may be solubilized in solutions containing high concentrations of urea (e.g., 8M) or chaotropic agents such as guanidine hydrochloride in the presence of reducing agents, such as β-mercaptoethanol or DTT (dithiothreitol), and refolded into a more desirable conformation, as would be known to one of ordinary skill in the art.

The nucleotide and protein, polypeptide and peptide sequences for various genes have been previously disclosed, and may be found at computerized databases known to those of ordinary skill in the art. One such database is the National Center for Biotechnology Information's Genbank and GenPept databases (www.ncbi.nlm.nih.gov/). The coding regions for these known genes may be amplified and/or expressed using the techniques disclosed herein or by any technique that would be know to those of ordinary skill in the art. Additionally, peptide sequences may be synthesized by methods known to those of ordinary skill in the art, such as peptide synthesis using automated peptide synthesis machines, such as those available from Applied Biosystems (Foster City, Calif.).

IV. METHODS OF MODULATING APOPTOSIS AND METHODS FOR SCREENING FOR MODULATORS OF U_{S}3, BAD ACTIVITY, AND APOPTOSIS

In one embodiment of the present invention, there are provided methods of screening compounds for activity against U_{S}3's anti-apoptotic activity. These screening methods will determine the cell pathology of target cells that express U_{S}3, both in the presence and absence of the test compound. In another embodiment, the present invention, provides methods of screening compounds for activity against ICP4's anti-apoptotic activity. At least three different assays may be employed, as discussed below.

First, one may look at DNA fragmentation using a separative method, e.g., chromatography or electrophoresis, to size fractionate the sample. As described in greater detail in the examples, an exemplary assay involves the isolation of DNA from cells, followed by agarose gel electrophoresis and staining with ethidium bromide. DNA fragmentation, characteristic of apoptosis, will be visualized as “ladders” containing a wide range of fragment sizes.

Second, one may employ terminal deoxynucleotidyl transferase mediated dUTP-biotin nick end labeling (TUNEL) assays to measure the integrity of DNA (Gorczyca, 1993). This assay measures the fragmentation of DNA by monitoring the incorporation of labeled UTP into broken DNA strands by the enzyme terminal transferase. The incorporation can be monitored by electroscopy or by cell sorting methodologies (e.g., FACS)

Third, one may examine cells using standard light or electron microscopy to assess the presence or absence of the cytopathologies characteristic of apoptosis. Those of skill in the art, applying standard methods of microscopy, will be able to assess cytopathology.

In each of these assays, a cell will be employed as the target for induction and inhibition of apoptosis. In one embodiment, the cell will be infected with HSV that expresses its own U_{S}3 protein. In a second embodiment, the cell will carry the U_{S}3 gene linked to a viral promoter. Infection with the appropriate virus will result in stimulation of the U_{S}3 gene and expression of U_{S}3. In these first two embodiments, the infection should induce apoptosis in the cell, and the expression of U_{S}3 should limit this effect. In a third embodiment, the cell will contain, as part of its own genetic material, an inducible version of the U_{S}3 gene (i.e., U_{S}3 linked to an inducible promoter). In this situation, it will be necessary to induce apoptosis via some other mechanism, such as hypothermia, osmotic shock or ICP4 expression, and express U_{S}3 by inducing the promoter. The present invention may further employ ICP4 alone or in combination with U_{S}3 in any of the embodiments described above.

The cell is contacted with a candidate inhibitor substance in order to assess its effect on U_{S}3 activity. The substance may be contacted with the cell prior to, at the same time, or after the provision of U_{S}3. In some cases, the candidate inhibitor substance may be contacted with the cell directly. In other situations, depending on the nature and putative mechanism of action, the candidate inhibitor substance may be reformulated to provide improved uptake. For example, where antisense oligonucleotides are provided, these may advantageously be formulated in liposomes or as virally-encapsulated expression vehicles. Where polypeptides are to be tested, it may be advantageous to provide expression vectors encoding these molecules rather than the polypeptides themselves. Essentially, the most reasonable mechanism for delivering an effective amount of the candidate inhibitor substance to the proper intracellular site will be chosen. “Effective amount,” for the purposes of the screening assay, is intended to mean an amount that will cause a detectable difference, and preferably a significant difference, in the cytopathology of the cell as compared to a similar treatment of the cell without the candidate inhibitor substance. A similar protocol may be used to test the effect of a candidate substance on ICP4 activity.

Once the candidate inhibitor substance has been provided to a cell that expresses the relevant peptide(s) (U_{S}3, ICP4 or both), the evaluation of cytopathology may be undertaken. Depending on the type of assay used to examine cytopathology, it is possible to automate this process and test hundreds of candidates at the same time. For example, 96-well trays may be employed in which several wells are reserved for controls while the remainder comprise test substances, usually with each substance being tested at several different amounts.

In certain embodiments, the present invention concerns a method for identifying modulators of U_{S}3 function or activity. Such modulators may be inhibitors or stimulators of such a function or activity. It is contemplated that this screening technique will prove useful in the general identification of any compound that will serve the purpose of inhibiting or stimulating U_{S}3-mediated kinase activity. In other embodiments a method for identifying modulators of BAD activity are also provided. Generally, the present invention provides methods for screening apoptosis modulators, that is, modulators of apoptosis. A “modulator” is a compound, substance, or agent that causes an alteration. An “apoptosis modulator” is such a compound, substance, or agent that causes an alteration with respect to apoptosis; an apoptosis modulator can inhibit, retard, prevent, reduce, increase, promote, induce, or trigger the frequency, rate, or incidence of apoptosis. In specific embodiments of the invention, an apoptosis modulator affects U_{S}3 so as to prevent it from inhibiting apoptosis, while in other embodiments U_{S}3 is the apoptosis modulator by being able to inhibit a cell from undergoing apoptosis.

The active compounds may include fragments or parts of naturally-occurring compounds or may be only found as active combinations of known compounds which are otherwise inactive. However, prior to testing of such compounds in humans or animal models, it will possibly be necessary to test a variety of candidates to determine which have potential.

Accordingly, in screening assays to identify pharmaceutical agents that modulate U_{S}3 kinase activity or BAD's pro-apoptotic activity, it is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds.

In these embodiments, the present invention is directed to a method for determining the ability of a candidate substance to inhibit or stimulate a protein kinase assay, the method including generally the steps of:

(a) obtaining an enzyme composition comprising a protein kinase, preferably U_{S}3 protein kinase that is capable of phosphorylating threonine/serine 1 5 residue;

(b) admixing a candidate substance with the enzyme composition; and

(c) determining the ability of the candidate substance to inhibit or stimulate threonine/serine phosphorylation.

In alternative embodiments, the present invention is directed to a method for determining the ability of a candidate substance to inhibit or stimulate BAD apoptotic activity, the method including generally:

(a) obtaining an proteinaceous composition comprising a pro-apoptotic activity, preferably BAD;

(b) admixing a candidate substance with the composition; and

(c) determining the ability of the candidate substance to inhibit or stimulate apoptosis of a cell.

To identify a candidate substance as being capable of modulating protein phosphorylation, one would first obtain an enzyme composition that is capable of phosphorylating threonine/serine residues on a protein of interest. Naturally, one would measure or determine the phosphorylation activity of the threonine/serine kinase composition in the absence of the added candidate substance. One would then add the candidate substance to the threonine/serine kinase composition and re-determine the ability of the threonine/serine kinase composition to phosphorylate threonine/serine residues on a test protein in the presence of the candidate substance. A candidate substance which reduces the phosphorylation activity of the threonine/serine kinase composition relative to the activity in its absence is indicative of a candidate substance with inhibitor capability. Likewise, a candidate substance which increases the phosphorylation activity of the threonine/serine kinase composition relative to the activity in its absence is indicative of a candidate substance with stimulatory capability. To identify a modulator of apoptosis, one would also compare the ability of a cell to undergo apoptosis in the presence and absence of the candidate substance. To perform the study, one may employ a cell that is prone to undergoing apoptosis or resistant to apoptosis. A cell may be rendered resistant to apoptosis by introducing all or part of a herpesvirus U_{S}3 polypeptide into a cell or using a cell transformed with such a polypeptide. Alternatively, a cell may be prone to undergoing apoptosis or induced to undergo apoptosis by introducing into the cell all or part of a BAD polypeptide or using a cell transformed with such a polypeptide. In other embodiments, a cell with a BAD polypeptide may be rendered resistant to apoptosis by introducing a compound that inhibits BAD activity. It is contemplated that the types of comparisons with respect to identifying kinase activity discussed above may be employed with respect to identifying modulators of apoptosis.

The candidate screening assay is quite simple to set up and perform, after obtaining a relatively purified preparation of the enzyme, either from native or recombinant sources, one will admix a candidate substance with the enzyme preparation, under conditions which would allow the enzyme to perform its threonine/serine phosphorylation function but for inclusion of a candidate substance. In this fashion, one can measure the ability of the candidate substance to reduce or increase the threonine/serine phosphorylation activity relatively in the presence of the candidate substance. Similarly, the candidate screening assay may be performed after obtaining a cell that can be assayed for characteristics of apoptosis in the presence and absence of a candidate compound under conditions that would allow apoptosis to occur.

“Effective amounts” in certain circumstances are those amounts effective to reproducibly reduce or increase threonine/serine kinase activity, or to inhibit or induce the apoptosis of cells, in comparison to their normal levels. Compounds that achieve significant appropriate changes in activity will be used. If desired, a battery of compounds may be screened in vitro to identify agents for use in the present invention.

Significant decrease in threonine/serine phosphorylation, e.g., as measured using immunoblotting techniques with anti-phosphorylation antibodies, are represented by a reduction in protein phosphorylation levels of at least about 30%-40%, and most preferably, by decreases of at least about 50%, with higher values of course being possible. Threonine/serine kinase assays that measure threonine/serine phosphorylation are well known in the art and may be conducted in vitro or in vivo. Likewise increases in threonine/serine phosphorylation are represented by an increase in protein phosphorylation levels of at least about 30%-40%, and most preferably, by increases of at least about 50%, with higher values of course being possible.

Assays for apoptosis will measure certain cellular events, including nuclear condensation, DNA fragmentation, cytoplasmic membrane blebbing and, ultimately, irreversible cell death. The methodology for such measurements is well known to those of skill in the art. A significant alteration in apoptosis is represented by an increase or decrease of at least about 30%-40% as compared to normal, and most preferably, of at least about 50%, with more significant increases or decreases also being possible. Therefore, if a candidate substance exhibited inhibition or induction of apoptosis in this type of study, it would likely be a suitable compound for use in the present invention.

Quantitative in vitro testing is not a requirement of the invention as it is generally envisioned that the candidate substance will often be selected on the basis of their known properties or by structural and/or functional comparison to those agents already known to have an effect on threonine/serine kinases.

It will, of course, be understood that all the screening methods of the present invention are useful in themselves notwithstanding the fact that effective candidates may not be found. The invention provides methods for screening for such candidates, not solely methods of finding them.

As used herein the term “candidate substance” refers to any molecule that may potentially modify apoptosis, including the activity of U_{S}3 or BAD. The candidate substance may inhibit or induce apoptosis activity or alter sensitivity to apoptosis inducers or inhibitors. The candidate substance may be a protein or fragment thereof, a small molecule, or even a nucleic acid molecule. An example of pharmacological compounds will be compounds that are structurally related to BAD, or a substrate of a U_{S}3. Using lead compounds to help develop improved compounds is know as “rational drug design” and includes not only comparisons with known inhibitors and activators, but predictions relating to the structure of target molecules. An “inhibitor” is a molecule which represses or prevents another molecule from engaging in a reaction. An “activator” is a molecule that increases the activity of an polypeptide or a protein that increases the production of a gene product in DNA transcription.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are well known to those of skill in the art. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

An inhibitor according to the present invention may be one which exerts its inhibitory or activating effect upstream, downstream or directly on compounds in an apoptosis pathway. Regardless of the type of inhibitor or activator identified by the present screening methods, the effect of the inhibition or activator by such a compound results in alteration in the disposition of a cell to undergo apoptosis as compared to that cell in the absence of the added candidate substance.

A. In vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

One example of a cell free assay is a binding assay. While not directly addressing function, the ability of a modulator to bind to a target molecule such as a U_{S}3 or BAD polypeptide in a specific fashion is strong evidence of a related biological effect. For example, binding of a molecule to a target may, in and of itself, be inhibitory, due to steric, allosteric or charge-charge interactions. The target may be either free in solution, fixed to a support, expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide can be detected by various methods.

B. In cyto Assays

The present invention also contemplates the screening of compounds for their ability to modulate apoptosis in cells. Various cell lines can be utilized for such screening assays, including cells specifically engineered for this purpose.

Depending on the assay, culture may be required. The cell is examined using any of a number of different physiologic assays. Alternatively, molecular analysis may be performed, for example, looking at protein expression, mRNA expression (including differential display of whole cell or polyA RNA) and others.

C. In vivo Assays

In vivo assays involve the use of various animal models, including transgenic animals that have been engineered to have specific defects, or carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal model derived from any of these species.

In such assays, one or more candidate substances are administered to an animal, and the ability of the candidate substance(s) to alter one or more characteristics, as compared to a similar animal not treated with the candidate substance(s), identifies a modulator. The characteristics may be any of those discussed above with regard to the function of a particular compound (e.g., enzyme, receptor, hormone) or cell (e.g., apoptosis, survival), or instead a broader indication such as behavior, anemia, immune response, recovery from viral infection, etc.

The present invention provides methods of screening for a candidate substance that is an apoptosis modulator. In these embodiments, the present invention is directed to a method for determining the ability of a candidate substance to induce or inhibit apoptosis in a cell, generally including the steps of: administering a candidate substance to the animal; and determining the ability of the candidate substance to modulate apoptosis.

Treatment of these animals with test compounds will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

Determining the effectiveness of a compound in vivo may involve a variety of different criteria. Also, measuring toxicity and dose response can be performed in animals in a more meaningful fashion than in in vitro or in cyto assays.

D. Methods for the Inhibition of Apoptosis

In one embodiment of the present invention, there are provided methods for the inhibition of apoptosis in a cell. This is particularly useful where one seeks to immortalize a cell or, at a minimum, increase the longevity of a cell. This permits one to maintain that cell in culture for extended periods of time, perhaps indefinitely. Immortalized cells are useful primarily as factories for production of viral vectors or proteins of interest, but it also may be important to immortalize cell simply so that they may be studied in vitro with greater ease. In addition, though many viruses provide promise as gene therapeutic vectors, these vectors may trigger apoptosis in the cells they infect. Blocking virally-induced apoptosis will prevent cell death caused by these therapeutic vectors. As mentioned above, adenovirus, papilloma viruses, retrovirus, adeno-associated virus and HSV, for example, are candidate gene therapeutic vectors that could benefit from this application.

The general approach to inhibiting apoptosis, according to the present invention, will be to provide a cell with an U_{S}3 polypeptide, an ICP4 polypeptide or both, thereby permitting the inhibitory activity of U_{S}3, ICP4 or both to take effect. While it is conceivable that the protein may be delivered directly, a preferred embodiment involves providing a nucleic acid encoding the polypeptide, i.e., an U_{S}3 gene, an α4 gene or both, to the cell. Following this provision, the polypeptide is synthesized by the host cell's transcriptional and translational machinery, as well as any that may be provided by the expression construct. Cis-acting regulatory elements necessary to support the expression of the U_{S}3 or α4 gene will be provided, as described above, in the form of an expression construct. It also is possible that, in the case of an HSV-infected cell, expression of the virally-encoded U_{S}3 or ICP4 could be stimulated or enhanced, or the expressed polypeptide stabilized, thereby achieving the same or similar effect.

In order to effect expression of constructs encoding U_{S}3 and/or ICP genes, the expression construct must be delivered into a cell. As described above in the discussion of viral vectors, one mechanism for delivery is via viral infection, where the expression construct is encapsidated in a viral particle which will deliver either a replicating or non-replicating nucleic acid. The preferred embodiment is an HSV vector, although virtually any vector would suffice. Similarly, where viral vectors are used to delivery other therapeutic genes, inclusion in these vectors of an U_{S}3 and/or α4 gene advantageously will protect the cell from virally induced apoptosis.

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et. al., 1990), and receptor-mediated transfection (Wu and Wu, 1987; Wu and Wu, 1988). Some of these techniques may be successfully adapted for in vivo or ex vivo use, as discussed below.

In another embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro, but it may be applied to in vivo use as well. Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al, 1990). The microprojectiles used have consisted of biologically inert substances such as tungsten or gold beads.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al (1980) demonstrated the feasibility of liposome-mediated delivery and expression of foreign DNA in cultured chick embryo, HeLa and hepatoma cells. In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In other embodiments, the delivery vehicle may comprise a ligand and a liposome. Where a bacterial promoter is employed in the DNA construct, it also will be desirable to include within the liposome an appropriate bacterial polymerase.

Other expression constructs which can be employed to deliver a nucleic acid encoding an U_{S}3 and/or α4 transgene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994). Mannose can be used to target the mannose receptor on liver cells. Also, antibodies to CD5 (CLL), CD22 (lymphoma), CD25 (T-cell leukemia) and MAA (melanoma) can similarly be used as targeting moieties. In other embodiments, the delivery vehicle may comprise a ligand and a liposome.

Primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented and are disclosed herein by reference (Freshner, 1992).

One embodiment of the foregoing involves the use of gene transfer to immortalize cells for the production of proteins. The gene for the protein of interest may be transferred as described above into appropriate host cells followed by culture of cells under the appropriate conditions. The gene for virtually any polypeptide may be employed in this manner. The generation of recombinant expression vectors, and the elements included therein, are discussed above. Alternatively, the protein to be produced may be an endogenous protein normally synthesized by the cell in question.

Examples of useful mammalian host cell lines are Vero and HeLa cells and cell lines of Chinese hamster ovary, W138, BHK, COS-7, 293, HepG2, 3T3, RIN and MDCK cells. In addition, a host cell strain may be chosen that modulates the expression of the inserted sequences, or modifies and process the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed.

A number of selection systems may be used including, but not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection for dhfr, that confers resistance to; gpt, that confers resistance to mycophenolic acid; neo, that confers resistance to the aminoglycoside G418; and hygro, that confers resistance to hygromycin.

Animal cells can be propagated in vitro in two modes: as non-anchorage dependent cells growing in suspension throughout the bulk of the culture or as anchorage-dependent cells requiring attachment to a solid substrate for their propagation (i.e., a monolayer type of cell growth).

Non-anchorage dependent or suspension cultures from continuous established cell lines are the most widely used means of large scale production of cells and cell products. However, suspension cultured cells have limitations, such as tumorigenic potential and lower protein production than adherent cells.

Large scale suspension culture of mammalian cells in stirred tanks is a common method for production of recombinant proteins. Two suspension culture reactor designs are in wide use—the stirred reactor and the airlift reactor. The stirred design has successfully been used on an 8000 liter capacity for the production of interferon (Phillips et al., 1985; Mizrahi, 1983). Cells are grown in a stainless steel tank with a height-to-diameter ratio of 1:1 to 3:1. The culture is usually mixed with one or more agitators, based on bladed disks or marine propeller patterns. Agitator systems offering less shear forces than blades have been described. Agitation may be driven either directly or indirectly by magnetically coupled drives. Indirect drives reduce the risk of microbial contamination through seals on stirrer shafts.

The airlift reactor, also initially described for microbial fermentation and later adapted for mammalian culture, relies on a gas stream to both mix and oxygenate the culture. The gas stream enters a riser section of the reactor and drives circulation. Gas disengages at the culture surface, causing denser liquid free of gas bubbles to travel downward in the downcomer section of the reactor. The main advantage of this design is the simplicity and lack of need for mechanical mixing. Typically, the height-to-diameter ratio is 10:1. The airlift reactor scales up relatively easily, has good mass transfer of gases and generates relatively low shear forces.

E. Methods for the Induction of Apoptosis

In another embodiment of the present invention, there is contemplated the method of inducing apoptosis in HSV-infected cells, i.e., blocking the apoptotic function of U_{S}3, ICP4 or both. In this way, it may be possible to curtail viral infection by bringing about a premature death of the infected cell. In addition, it may prove effective to use this sort of therapeutic intervention in combination with more traditional chemotherapies, such as the administration acyclovir.

The general form that this aspect of the invention will take is the provision, to a cell, of an agent that will inhibit U_{S}3 function, ICP4 function or indeed inhibit the function of both. A number of such agents are contemplated as outlined below.

First, one may employ an antisense nucleic acid that will hybridize either to the U_{S}3 gene or the U_{S}3 transcript, thereby preventing transcription or translation, respectively. Likewise, one may employ an antisense nucleic acid that will hybridize to an α4 gene or an α4 transcript. The considerations relevant to the design of antisense constructs have been presented above.

Second, one may utilize an U_{S}3-binding protein or peptide, for example, a peptidomimetic or an antibody that binds immunologically to an U_{S}3, the binding of either will block or reduce the activity of an U_{S}3. Again the same is true of ICP4 in that one may utilize an ICP4-binding protein or peptide, for example, a peptidomimetic or an antibody that binds immunologically to an ICP4, the binding of either resulting in a block or reduction of ICP4 activity. The methods of making and selecting peptide binding partners and antibodies are well known to those of skill in the art.

Third, one may provide to the cell an antagonist of U_{S}3 for example, the in the case of U_{S}3 phosphorylation target sequence, alone or coupled to another agent. Equally one may provide to the cell an antagonist of ICP4, for example, the transactivation target sequence, alone or coupled to another agent. And fourth, one may provide an agent that binds to the U_{S}3 or ICP4 target without the same functional result as would arise with U_{S}3 or ICP4 binding.

Moreover, one may provide all or part of a BAD polypeptide to a cell, which could induce apoptosis. Alternatively, in a cell infected with a herpesvirus or having a U_{S}3 polypeptide, an inhibitor of U_{S}3 may be provided to prevent its effect on BAD. Such an inhibitor may prevent U_{S}3 from post-translationally modifying BAD or may prevent the reduction of BAD protein levels in cells containing U_{S}3.

Provision of an U_{S}3 gene or encoding nucleic acid, an U_{S}3-binding protein, a U_{S}3 polypeptide or peptide, an U_{S}3 antagonist, an U_{S}3 modulator, or an α4 gene, BAD peptide or polypeptide, BAD-encoding nucleic acid, a BAD modulator, an ICP4-binding protein, or an ICP4 antagonist, would be according to any appropriate pharmaceutical route. The formulation of such compositions and their delivery to tissues is discussed below. The method by which the nucleic acid, protein or chemical is transferred, along with the preferred delivery route, will be selected based on the particular site to be treated. Those of skill in the art are capable of determining the most appropriate methods based on the relevant clinical considerations.

Many of the gene transfer techniques that generally are applied in vitro can be adapted for ex vivo or in vivo use. For example, selected organs including the liver, skin, and muscle tissue of rats and mice have been bombarded in vivo (Yang et al., 1990; Zelenin et al., 1991). Naked DNA also has been used in clinical settings to effect gene therapy. These approaches may require surgical exposure of the tumor tissue or direct intratumoral injection. Nicolau et al. (1987) accomplished successful liposome-mediated gene transfer in rats after intravenous injection.

Dubensky et al. (1984) successfully injected polyomavirus DNA in the form of CaPO₄ precipitates into liver and spleen of adult and newborn mice demonstrating active viral replication and acute infection. Benvenisty and Neshif (1986) also demonstrated that direct intraperitoneal injection of CaPO₄ precipitated plasmids results in expression of the transfected genes. Thus, it is envisioned that DNA encoding an antisense construct also may be transferred in a similar manner in vivo.

Where the embodiment involves the use of an antibody that recognizes an U_{S}3 or an ICP4 polypeptide, consideration must be given to the mechanism by which the antibody is introduced into the cell cytoplasm. This can be accomplished, for example, by providing an expression construct that encodes a single-chain antibody version of the antibody to be provided. Most of the discussion above relating to expression constructs for antisense versions of the respective genes will be relevant to this aspect of the invention. Alternatively, it is possible to present a bifunctional antibody, where one antigen binding arm of the antibody recognizes an U_{S}3 or ICP4 polypeptide and the other antigen binding arm recognizes a receptor on the surface of the cell to be targeted. Examples of suitable receptors would be an HSV glycoprotein such as gB, gC, gD, or gH. In addition, it may be possible to exploit the Fc-binding function associated with HSV gE, thereby obviating the need to sacrifice one arm of the antibody for purposes of cell targeting.

Advantageously, one may combine this approach with more conventional chemotherapeutic options. Acyclovir is an active agent against HSV-1 and HSV-2. The drug inhibits actively replicating herpes virus but is not active against latent virus. Acyclovir is available in three formulations. For topical use, a five percent ointment produces therapeutic drug levels in mucocutaneous lesions. For systemic use, acyclovir may be administered orally or intravenously. The usual intravenous dosage in adults with normal renal function is 5 mg/kg infused at a constant rate over one hour and given every eight hours; this dosage produces peak plasma levels at about 10 g/ml. For HSV encephalitis, twice this dose is used. The usual adult oral dosage is 200 mg, five times daily, which produces plasma levels that are less than 10% as high as those achieved with intravenous administration; even these levels are inhibitory to the virus, however. Acyclovir is given in an oral dosage of 800 mg five times daily for the treatment of herpes zoster, although oral administration generally is reserved for patients with severe symptoms. A three percent opthalmic preparation produces inhibitory drug levels in the aqueous humor and is effective for herpes keratitis.

In contemplating combinatorial aspects of the present invention, it will be possible to combine the U_{S}3 therapeutic compositions of the present invention with other apoptosis inducing or inhibiting compositions. For example the present inventors have shown that it is possible to affect apoptosis using compositions derived from ICP4 protein or polypeptide and the α4 gene or gene constructs. Thus in light of the teachings presented herein, it will be possible for one of ordinary skill in the art to combine the U_{S}3 therapeutic compositions with those derived from ICP4 and α4 to affect apoptosis as described herein below. Alternatively, a combination may be employed using a BAD peptide or polypeptide, or some other apoptosis modulator. In further embodiments, anti-viral compounds may be employed such as famcyclovir, valacyclovir, or acyclovir.

To kill cells, inhibit cell growth, inhibit apoptosis, or induce apoptosis as defined above, using the methods and compositions of the present invention, one would generally contact a “target” cell with a U_{S}3 and/or an ICP4 expression construct alone or in combination with at least one other agent. These compositions would be provided in a combined amount effective to inhibit or to kill or induce apoptosis of the cell. This process may involve contacting the cells with the expression construct and the agent(s) or factor(s) at the same time. This may be achieved by contacting the cell with a single composition or pharmacological formulation that includes both agents, or by contacting the cell with two distinct compositions or formulations, at the same time, wherein one composition includes the expression construct and the other includes the agent.

Alternatively, the gene therapy treatment may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either U_{S}3 or the other agent will be desired. Various combinations may be employed, where U_{S}3 is “A” and the other agent is “B”, as exemplified below:

A/B/A B/A/B B/B/A A/A/B B/A/A A/B/B B/B/B/A B/B/A/B

A/A/B/B A/B/A/B A/B/B/A B/B/A/A B/A/B/A B/A/A/B B/B/B/A

A/A/A/B B/A/A/A A/B/A/A A/A/B/A A/B/B/B B/A/B/B B/B/A/B

Other combinations are contemplated where ICP4 and α4 may also be used, or a BAD peptide or polypeptide, or other apoptosis modulator. Again, to achieve apoptosis, both agents are delivered to a cell in a combined amount effective induce cell death. To inhibit apoptosis, multiple agents are delivered to a cell in a combined amount effective to inhibit apoptosis for more than 1, 2, 3, 4, 5, 6, 7, 8, 9, 10, 11, 12, 13, 14, 15, 16, 17, 18, 19, 20, or more days.

The skilled artisan is directed to “Remington's Pharmaceutical Sciences” 15th Edition, chapter 33, in particular pages 624-652. Some variation in dosage will necessarily, occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The inventors propose that the regional delivery of U_{S}3 expression constructs and/or ICP4 expression constructs to patients with HSV linked disease will be a very efficient method for delivering a therapeutically effective gene to counteract the clinical disease. Similarly, the chemotherapy may be directed to a particular, affected region of the subjects body. Alternatively, systemic delivery of expression construct and/or the agent may be appropriate in certain circumstances, for example, where extensive metastasis has occurred.

In addition to combining U_{S}3-targeted therapies with chemotherapies, it also is contemplated that combination with other gene therapies will be advantageous. For example, targeting of U_{S}3 and α4 mutations at the same time may produce an improved apoptotic treatment.

It also should be pointed out that any of the foregoing therapies may prove useful by themselves in treating a U_{S}3 related disease. In this regard, reference to chemotherapeutics and non-U_{S}3 gene therapy in combination should also be read as a contemplation that these approaches may be employed separately.

F. Methods for the Inhibition of Virus-Induced Cell Death In Vivo

In another embodiment of the present invention, there are provided methods for the inhibition of cell death induced in vivo by any cause comprising the provision of U_{S}3 polypeptides or U_{S}3 genes, or ICP4 polypeptides and α4 genes or combinations of the two. Alternatively, other embodiments are contemplated in which inhibition of apoptosis that might otherwise by induced through BAD. Also contemplated in this aspect of the invention is the stimulation of viral U_{S}3 expression, and/or stimulation of viral ICP4 expression or stabilization of the virally-expressed U_{S}3 polypeptide and or ICP4 polypeptide. Though inhibition of apoptosis generally is thought of as advantageous to the virus, it may be desirable to effect this result as part of a method of treating a viral infection. For example, if the host cell remains viable, the virus may continue to replicate; alternatively, if apoptosis were occurring, the virus might be inclined to “go latent” in the neural ganglia, where chemotherapeutic intervention is not helpful. Thus, by preventing early death of the cell, U_{S}3 and/or ICP4 may cause the virus to remain susceptible to treatment where it otherwise would escape.

The mechanisms for delivering proteins and nucleic acids to a cell are discussed elsewhere in this document and need not be repeated here. The use of standard chemotherapeutics or antiviral treatments has been presented in the preceding section, and is incorporated in this section.

### V. PHARMACEUTICALS AND IN VIVO METHODS OF THE TREATMENT OF DISEASE

Aqueous pharmaceutical compositions of the present invention will have an effective amount of an U_{S}3, BAD, and/or α4 expression construct, an antisense U_{S}3 and/or α4 expression construct, an expression construct that encodes a therapeutic gene along with U_{S}3 and/or α4, a protein that inhibits U_{S}3 and/or α4 function, such as an anti-U_{S}3 antibody or an anti-ICP4 antibody, respectively, or an U_{S}3 polypeptide and/or an ICP4 polypeptide. Alternatively, an effective amount of a BAD peptide or polypeptide, a BAD-encoding nucleic acid, a BAD antisense or ribozyme molecule, or an apotosis modulator-inhibitors or inducers-may also be formulated in a pharmaceutical composition. Such compositions generally will be dissolved or dispersed in a pharmaceutically acceptable carrier or aqueous medium. An “effective amount,” for the purposes of therapy, is defined at that amount that causes a clinically measurable difference in the condition of the subject. This amount will vary depending on the substance, the condition of the patient, the type of treatment, the location of the lesion, etc.

The phrases “pharmaceutically or pharmacologically acceptable” refer to molecular entities and compositions that do not produce an adverse, allergic or other untoward reaction when administered to an animal, or human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredients, its use in the therapeutic compositions is contemplated. Supplementary active ingredients, such as other anti-cancer agents, can also be incorporated into the compositions.

In addition to the compounds formulated for parenteral administration, such as those for intravenous or intramuscular injection, other pharmaceutically acceptable forms include, e.g., tablets or other solids for oral administration; time release capsules; and any other form currently used, including cremes, lotions, mouthwashes, inhalants and the like.

The active compounds of the present invention will often be formulated for parenteral administration, e.g., formulated for injection via the intravenous, intramuscular, subcutaneous, or even intraperitoneal routes. The preparation of an aqueous composition that contains glycosylceramide synthesis inhibitory compounds alone or in combination with a chemotherapeutic agent as active ingredients will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In many cases, the form must be sterile and must be fluid to the extent that easy syringability exists. It must be stable under the conditions of manufacture and storage and must be preserved against the contaminating action of microorganisms, such as bacteria and fungi.

The active compounds may be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In certain cases, the therapeutic formulations of the invention could also be prepared in forms suitable for topical administration, such as in cremes and lotions. These forms may be used for treating skin-associated diseases, such as various sarcomas. In certain other cases, the formulation will be geared for administration to the central nervous system, e.g., the brain.

Upon formulation, solutions will be administered in a manner compatible with the dosage formulation and in such amount as is therapeutically effective. The formulations are easily administered in a variety of dosage forms, such as the type of injectable solutions described above, with even drug release capsules and the like being employable.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

A. Lipid Compositions

In certain embodiments, the present invention concerns a novel composition comprising one or more lipids associated with a polynucleotide or polypeptide of the claimed invention. A lipid is a substance that is characteristically insoluble in water and extractable with an organic solvent. Compounds than those specifically described herein are understood by one of skill in the art as lipids, and are encompassed by the compositions and methods of the present invention.

A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glucolipids, sulphatides, lipids with ether and ester-linked fatty acids and polymerizable lipids, and combinations thereof.

1. Lipid Types

A neutral fat may comprise a glycerol and a fatty acid. A typical glycerol is a three carbon alcohol. A fatty acid generally is a molecule comprising a carbon chain with an acidic moiety (e.g., carboxylic acid) at an end of the chain. The carbon chain may of a fatty acid may be of any length, however, it is preferred that the length of the carbon chain be of from about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, to about 30 or more carbon atoms, and any range derivable therein. However, a preferred range is from about 14 to about 24 carbon atoms in the chain portion of the fatty acid, with about 16 to about 18 carbon atoms being particularly preferred in certain embodiments. In certain embodiments the fatty acid carbon chain may comprise an odd number of carbon atoms, however, an even number of carbon atoms in the chain may be preferred in certain embodiments. A fatty acid comprising only single bonds in its carbon chain is called saturated, while a fatty acid comprising at least one double bond in its chain is called unsaturated.

Specific fatty acids include, but are not limited to, linoleic acid, oleic acid, palmitic acid, linolenic acid, stearic acid, lauric acid, myristic acid, arachidic acid, palmitoleic acid, arachidonic acid ricinoleic acid, tuberculosteric acid, lactobacillic acid. An acidic group of one or more fatty acids is covalently bonded to one or more hydroxyl groups of a glycerol. Thus, a monoglyceride comprises a glycerol and one fatty acid, a diglyceride comprises a glycerol and two fatty acids, and a triglyceride comprises a glycerol and three fatty acids.

A phospholipid generally comprises either glycerol or an sphingosine moiety, an ionic phosphate group to produce an amphipathic compound, and one or more fatty acids. Types of phospholipids include, for example, phophoglycerides, wherein a phosphate group is linked to the first carbon of glycerol of a diglyceride, and sphingophospholipids (e.g., sphingomyelin), wherein a phosphate group is esterified to a sphingosine amino alcohol. Another example of a sphingophospholipid is a sulfatide, which comprises an ionic sulfate group that makes the molecule amphipathic. A phopholipid may, of course, comprise further chemical groups, such as for example, an alcohol attached to the phosphate group. Examples of such alcohol groups include serine, ethanolamine, choline, glycerol and inositol. Thus, specific phosphoglycerides include a phosphatidyl serine, a phosphatidyl ethanolamine, a phosphatidyl choline, a phosphatidyl glycerol or a phosphotidyl inositol. Other phospholipids include a phosphatidic acid or a diacetyl phosphate. In one aspect, a phosphatidylcholine comprises a dioleoylphosphatidylcholine (a.k.a. cardiolipin), an egg phosphatidylcholine, a dipalmitoyl phosphalidycholine, a monomyristoyl phosphatidylcholine, a monopalmitoyl phosphatidylcholine, a monostearoyl phosphatidylcholine, a monooleoyl phosphatidylcholine, a dibutroyl phosphatidylcholine, a divaleroyl phosphatidylcholine, a dicaproyl phosphatidylcholine, a diheptanoyl phosphatidylcholine, a dicapryloyl phosphatidylcholine or a distearoyl phosphatidylcholine.

A glycolipid is related to a sphinogophospholipid, but comprises a carbohydrate group rather than a phosphate group attached to a primary hydroxyl group of the sphingosine. A type of glycolipid called a cerebroside comprises one sugar group (e.g., a glucose or galactose) attached to the primary hydroxyl group. Another example of a glycolipid is a ganglioside (e.g., a monosialoganglioside, a GM1), which comprises about 2, about 3, about 4, about 5, about 6, to about 7 or so sugar groups, that may be in a branched chain, attached to the primary hydroxyl group. In other embodiments, the glycolipid is a ceramide (e.g., lactosylceramide).

A steroid is a four-membered ring system derivative of a phenanthrene. Steroids often possess regulatory functions in cells, tissues and organisms, and include, for example, hormones and related compounds in the progestagen (e.g., progesterone), glucocoricoid (e.g., cortisol), mineralocorticoid (e.g., aldosterone), androgen (e.g., testosterone) and estrogen (e.g., estrone) families. Cholesterol is another example of a steroid, and generally serves structural rather than regulatory functions. Vitamin D is another example of a sterol, and is involved in calcium absorption from the intestine.

A terpene is a lipid comprising one or more five carbon isoprene groups. Terpenes have various biological functions, and include, for example, vitamin A, coenyzme Q and carotenoids (e.g., lycopene and β-carotene).

2. Charged and Neutral Lipid Compositions

In certain embodiments, a lipid component of a composition is uncharged or primarily uncharged. In one embodiment, a lipid component of a composition comprises one or more neutral lipids. In another aspect, a lipid component of a composition may be substantially free of anionic and cationic lipids, such as certain phospholipids and cholesterol. In certain aspects, a-lipid component of an uncharged or primarily uncharged lipid composition comprises about 95%, about 96%, about 97%, about 98%, about 99% or 100% lipids without a charge, substantially uncharged lipid(s), and/or a lipid mixture with equal numbers of positive and negative charges.

In other aspects, a lipid composition may be charged. For example, charged phospholipids may be used for preparing a lipid composition according to the present invention and can carry a net positive charge or a net negative charge. In a non-limiting example, diacetyl phosphate can be employed to confer a negative charge on the lipid composition, and stearylamine can be used to confer a positive charge on the lipid composition.

3. Making Lipids

Lipids can be obtained from natural sources, commercial sources or chemically synthesized, as would be known to one of ordinary skill in the art. For example, phospholipids can be from natural sources, such as egg or soybean phosphatidylcholine, brain phosphatidic acid, brain or plant phosphatidylinositol, heart cardiolipin and plant or bacterial phosphatidylethanolamine. In another example, lipids suitable for use according to the present invention can be obtained from commercial sources. For example, dimyristyl phosphatidylcholine (“DMPC”) can be obtained from Sigma Chemical Co., dicetyl phosphate (“DCP”) is obtained from K & K Laboratories (Plainview, N.Y.); cholesterol (“Chol”) is obtained from Calbiochem-Behring; dimyristyl phosphatidylglycerol (“DMPG”) and other lipids may be obtained from Avanti Polar Lipids, Inc. (Birmingham, Ala.). In certain embodiments, stock solutions of lipids in chloroform or chloroform/methanol can be stored at about −20° C. Preferably, chloroform is used as the only solvent since it is more readily evaporated than methanol.

4. Lipid Composition Structures

A compound associated with a lipid may be dispersed in a solution containing a lipid, dissolved with a lipid, emulsified with a lipid, mixed with a lipid, combined with a lipid, covalently bonded to a lipid, contained as a suspension in a lipid or otherwise associated with a lipid. A lipid or lipid-associated composition of the present invention is not limited to any particular structure. For example, they may also simply be interspersed in a solution, possibly forming aggregates which are not uniform in either size or shape. In another example, they may be present in a bilayer structure, as micelles, or with a “collapsed” structure. In another non-limiting example, a lipofectamine(Gibco BRL) or Superfect (Qiagen) complex is also contemplated.

In certain embodiments, a lipid composition may comprise about 1%, about 2%, about 3%, about 4% about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100%, or any range derivable therein, of a particular lipid, lipid type or non-lipid component such as a drug, protein, sugar, nucleic acids or other material disclosed herein or as would be known to one of skill in the art. In a non-limiting example, a lipid composition may comprise about 10% to about 20% neutral lipids, and about 33% to about 34% of a cerebroside, and about 1% cholesterol. In another non-limiting example, a liposome may comprise about 4% to about 12% terpenes, wherein about 1% of the micelle is specifically lycopene, leaving about 3% to about 11% of the liposome as comprising other terpenes; and about 10%to about 35% phosphatidyl choline, and about 1% of a drug. Thus, it is contemplated that lipid compositions of the present invention may comprise any of the lipids, lipid types or other components in any combination or percentage range.

a. Emulsions

A lipid may be comprised in an emulsion. A lipid emulsion is a substantially permanent heterogenous liquid mixture of two or more liquids that do not normally dissolve in each other, by mechanical agitation or by small amounts of additional substances known as emulsifiers. Methods for preparing lipid emulsions and adding additional components are well known in the art (e.g., Modem Pharmaceutics, 1990, incorporated herein by reference).

For example, one or more lipids are added to ethanol or chloroform or any other suitable organic solvent and agitated by hand or mechanical techniques. The solvent is then evaporated from the mixture leaving a dried glaze of lipid. The lipids are resuspended in aqueous media, such as phosphate buffered saline, resulting in an emulsion. To achieve a more homogeneous size distribution of the emulsified lipids, the mixture may be sonicated using conventional sonication techniques, further emulsified using microfluidization (using, for example, a Microfluidizer, Newton, Mass.), and/or extruded under high pressure (such as, for example, 600 psi) using an Extruder Device (Lipex Biomembranes, Vancouver, Canada).

b. Micelles

A lipid may be comprised in a micelle. A micelle is a cluster or aggregate of lipid compounds, generally in the form of a lipid monolayer, and may be prepared using any micelle producing protocol known to those of skill in the art (e.g., Canfield et al., 1990; El-Gorab et al, 1973; Colloidal Surfactant, 1963; and Catalysis in Micellar and Macromolecular Systems, 1975, each incorporated herein by reference). For example, one or more lipids are typically made into a suspension in an organic solvent, the solvent is evaporated, the lipid is resuspended in an aqueous medium, sonicated and then centrifuged.

5. Liposomes

In particular embodiments, a lipid comprises a liposome. A “liposome” is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition.

A multilamellar liposome has multiple lipid layers separated by aqueous medium. They form spontaneously when lipids comprising phospholipids are suspended in an excess of aqueous solution. The lipid components undergo self-rearrangement before the formation of closed structures and entrap water and dissolved solutes between the lipid bilayers (Ghosh and Bachhawat, 1991). Lipophilic molecules or molecules with lipophilic regions may also dissolve in or associate with the lipid bilayer.

In specific aspects, a lipid and/or modified protein or polynucleotide encoding a modified protein may be, for example, encapsulated in the aqueous interior of a liposome, interspersed within the lipid bilayer of a liposome, attached to a liposome via a linking molecule that is associated with both the liposome and the composition, entrapped in a liposome, complexed with a liposome, etc.

a. Making Liposomes

A liposome used according to the present invention can be made by different methods, as would be known to one of ordinary skill in the art.

For example, a phospholipid (Avanti Polar Lipids, Alabaster, Ala.), such as for example the neutral phospholipid dioleoylphosphatidylcholine (DOPC), is dissolved in tert-butanol. The lipid(s) is then mixed with the polynucleotide or polypeptide, and/or other component(s). Tween 20 is added to the lipid mixture such that Tween 20 is about 5% of the composition's weight. Excess tert-butanol is added to this mixture such that the volume of tert-butanol is at least 95%. The mixture is vortexed, frozen in a dry ice/acetone bath and lyophilized overnight. The lyophilized preparation is stored at −20° C. and can be used up to three months. When required the lyophilized liposomes are reconstituted in 0.9% saline. The average diameter of the particles obtained using Tween 20 for encapsulating the compound is about 0.7 to about 1.0 μm in diameter.

Alternatively, a liposome can be prepared by mixing lipids in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min. to 2 hours, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

In other alternative methods, liposomes can be prepared in accordance with other known laboratory procedures (e.g., see Bangham et al., 1965; Gregoriadis, 1979; Deamer and Uster 1983, Szoka and Papahadjopoulos, 1978, each incorporated herein by reference in relevant part). These methods differ in their respective abilities to entrap aqueous material and their respective aqueous space-to-lipid ratios.

The dried lipids or lyophilized liposomes prepared as described above may be dehydrated and reconstituted in a solution of inhibitory peptide and diluted to an appropriate concentration with an suitable solvent, e.g. DPBS. The mixture is then vigorously shaken in a vortex mixer. Unencapsulated additional materials, such as agents including but not limited to hormones, drugs, nucleic acid constructs and the like, are removed by centrifugation at 29,000×g and the liposomal pellets washed. The washed liposomes are resuspended at an appropriatetotal phospholipid concentration, e.g., about 50-200 mM. The amount of additional material or active agent encapsulated can be determined in accordance with standard methods. After determination of the amount of additional material or active agent encapsulated in the liposome preparation, the liposomes may be diluted to appropriate concentrations and stored at 4° C. until use. A pharmaceutical composition comprising the lipsomes will usually include a sterile, pharmaceutically acceptable carrier and diluent, such as water saine solution.

The size of a liposome varies depending on the method of synthesis. Liposomes in the present invention can be a variety of sizes. In certain embodiments, the lipsomes are small, e.g., less than about 100 nm, about 90 nm, about 80 nm, about 70 nm, about 60 nm, or less than about 50 nm in external diameter. In preparing such liposomes, any protocol described herein, or as would be known to one of ordinary skill in the art may be used. Additional non-limiting examples of examples of preparing liposomes are described U.S. Pat. Nos. 4,728,578, 4,728,575, 4,737,323, 4,533,254 4,162,282 4,310,505, and 4,921,706; International Applications PCT/US85/01161 and PCT/US89/05040; U.K. Patent Application GB 2193095 A; Meyer et al., 19876; Hope et al., 1985; Mayhew et al. 1987; Mayhew et al., 1984; Cheng et al., 1987; and Liposome Technology, 1984, each incorporated herein by reference).

Liposome suspended in an aqueous solution is generally in the shape of a spherical vesicle, having one or more concentric layers of lipid bilayer molecules. Each layer consists of a parallel array of molecules represented by the formula XY, wherein X is a hydrophilic moiety and Y is a hydrophobic moiety. In aqueous suspension, the concentric layers are arranged such that the hydrophilic moieties tend to remain in contact with an aqueous phase and the hydrophobic regions tend to self-associate. For example, when aqueous phases are present both within and without the liposome, the lipid molecules may form a bilayer, known as a lamella, of the arrangement XY-YX. Aggregates of lipids may form when the hydrophilic and hydrophobic parts of more than one lipid molecule become associated with each other. The size and shape of these aggregates will depend upon many different variables, such as the nature of the solvent and the presence of other compounds in the solution.

The production of lipid formulations often is accomplished by sonication or serial extrusion of liposomal mixtures after (I) reverse phase evaporation (II) dehydration-rehydration (III) detergent dialysis and (IV) thin film hydration. In one aspect, a contemplated method for preparing liposomes in certain embodiments is heating sonicating, and sequential extrusion of the lipids through filters or membranes of decreasing pore size, thereby resulting in the formation of small, stable liposome structures. This preparation produces liposomal/therapeutic compound or liposomes only of appropriate and uniform size, which are structurally stable and produce maximal activity. Such techniques are well-known to those of skill in the art (see, for example Martin, 1990).

Once manufactured, lipid structures can be used to encapsulate compounds that are toxic (e.g., chemotherapeutics) or labile (e.g., nucleic acids) when in circulation. Liposomal encapsulation has resulted in a lower toxicity and a longer serum half-life for such compounds (Gabizon et al., 1990).

Numerous disease treatments are using lipid based gene transfer strategies to enhance conventional or establish novel therapies, in particular therapies for treating hyperproliferative diseases. Advances in liposome formulations have improved the efficiency of gene transfer in vivo (Templeton et al., 1997) and it is contemplated that liposomes are prepared by these methods. Alternate methods of preparing lipid-based formulations for nucleic acid delivery are described (WO 99/18933).

In another liposome formulation, an amphipathic vehicle called a solvent dilution microcarrier (SDMC) enables integration of particular molecules into the bi-layer of the lipid vehicle (U.S. Pat. No. 5,879,703). The SDMCs can be used to deliver lipopolysaccharides, polypeptides, nucleic acids and the like. Of course, any other methods of liposome preparation can be used by the skilled artisan to obtain a desired liposome formulation in the present invention.

b. Liposome Targeting

Association of the compositions of the invention with a liposome may improve its biodistribution and other properties. For example, liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980). Successful liposome-mediated gene transfer in rats after intravenous injection has also been accomplished (Nicolau et al., 1987).

It is contemplated that a liposomecomposition may comprise additional materials for delivery to a tissue. For example, in certain embodiments of the invention, the lipid or liposome may be associated with a hemagglutinating virus (HVJ). This has been shown to facilitate fusion with the cell membrane and promote cell entry of liposome-encapsulated DNA (Kaneda et al., 1989). In another example, the lipid or liposome may be complexed or employed in conjunction with nuclear non-histone chromosomal proteins (HMG-1) (Kato et al., 1991). In yet further embodiments, the lipid may be complexed or employed in conjunction with both HVJ and HMG-1.

Targeted delivery is achieved by the addition of ligands without compromising the ability of these liposomes deliver large amounts of any disclosed compound of the invention It is contemplated that this will enable delivery to specific cells, tissues and organs. The targeting specificity of the ligand-based delivery systems are based on the distribution of the ligand receptors on different cell types. The targeting ligand may either be non-covalently or covalently associated with the lipid complex, and can be conjugated to the liposomes by a variety of methods.

Exemplary methods for cross-linking ligands (some discussed above) to liposomes are described in U.S. Pat. No. 5,603,872 and U.S. Pat. No. 5,401,511, each specifically incorporated herein by reference in its entirety). Various ligands can be covalently bound to liposomal surfaces through the cross-linking of amine residues. Liposomes, in particular, multilamellar vesicles (MLV) or unilamellar vesicles such as microemulsified liposomes (MEL) and large unilamellar liposomes (LUVET), each containing phosphatidylethanolamine (PE), have been prepared by established procedures. The inclusion of PE in the liposome provides an active functional residue, a primary amine, on the liposomal surface for cross-linking purposes. Ligands such as epidermal growth factor (EGF) have been successfully linked with PE-liposomes. Ligands are bound covalently to discrete sites on the liposome surfaces. The number and surface density of these sites will be dictated by the liposome formulation and the liposome type. The liposomal surfaces may also have sites for non-covalent association. To form covalent conjugates of ligands and liposomes, cross-linking reagents have been studied for effectiveness and biocompatibility. Cross-linking reagents include glutaraldehyde (GAD), bifunctional oxirane (OXR), ethylene glycol diglycidyl ether (EGDE), and a water soluble carbodlimide, preferably 1-ethyl-3-(3-dimethylaminopropyl)carbodiimide (EDC). Through the complex chemistry of cross-linking, linkage of the amine residues of the recognizing substance and liposomes is established.

i. Targeting Ligands

The targeting ligand can be either anchored in the hydrophobic portion of the complex or attached to reactive terminal groups of the hydrophilic portion of the complex. The targeting ligand can be attached to the liposome via a linkage to a reactive group, e.g., on the distal end of the hydrophilic polymer. Preferred reactive groups include amino groups, carboxylic groups, hydrazide groups, and thiol groups. The coupling of the targeting ligand to the hydrophilic polymer can be performed by standard methods of organic chemistry that are known to those skilled in the art. In certain embodiments, the total concentration of the targeting ligand can be from about 0.01 to about 10% mol.

Targeting ligands are any ligand specific for a characteristic component of the targeted region. Preferred targeting ligands include proteins such as polyclonal or monoclonal antibodies, antibody fragments, or chimeric antibodies, enzymes, or hormones, or sugars such as mono-, oligo- and poly-saccharides (see, Heath et al., 1986) For example, disialoganglioside GD2 is a tumor antigen that has been identified neuroectodermal origin tumors, such as neuroblastoma, melanoma, small-cell lung carcenoma, glioma and certain sarcomas (Cheresh et al., 1986; Schulz et al., 1984). Liposomes containing anti-disialoganglioside GD2 monoclonal antibodies have been used to aid the targeting of the liposomes to cells expressing the tumor antigen (Montaldo et al., 1999; Pagnan et al., 1999). In another non-limiting example, breast and gynecological cancer antigen specific antibodies are described in U.S. Pat. No. 5,939,277, incorporated herein by reference. In a further non-limiting example, prostate cancer specific antibodies are disclosed in U.S. Pat. No. 6,107,090, incorporated herein by reference. Thus, it is contemplated that the antibodies described herein or as would be known to one of ordinary skill in the art may be used to target specific tissues and cell types in combination with the compositions and methods of the present invention. In certain embodiments of the invention, contemplated targeting ligands interact with integrins, proteoglycans, glycoproteins, receptors or transporters. Suitable ligands include any that are specific for cells of the target organ, or for structures of the target organ exposed to the circulation as a result of local pathology, such as tumors.

In certain embodiments of the present invention, in order to enhance the transduction of cells, to increase transduction of target cells, or to limit transduction of undesired cells, antibody or cyclic peptide targeting moieties (ligands) are associated with the lipid complex. Such methods are known in the art. For example, liposomes have been described further that specifically target cells of the mammalian central nervous system (U.S. Pat. No. 5,786,214, incorporated herein by reference). The liposomes are composed essentially of N-glutarylphosphatidylethanolamine, cholesterol and oleic acid, wherein a monoclonal antibody specific for neuroglia is conjugated to the liposomes. It is contemplated that a monoclonal antibody or antibody fragment may be used to target delivery to specific cells, tissues, or organs in the animal, such as for example, brain, heart, lung, liver, etc.

Still further, a compound may be delivered to a target cell via receptor-mediated delivery and/or targeting vehicles comprising a lipid or liposome. These take advantage of the selective uptake of macromolecules by receptor-mediated endocytosis that will be occurring in a target cell. In view of the cell type-specific distribution of various receptors, this delivery method adds another degree of specificity to the present invention.

Thus, in certain aspects of the present invention, a ligand will be chosen to correspond to a receptor specifically expressed on the target cell population. A cell-specific delivery of compounds of the invention and/or targeting vehicle may comprise a specific binding ligand in combination with a liposome. The compounds to be delivered are housed within a liposome and the specific binding ligand is functionally incorporated into a liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In certain embodiments, a receptor-mediated delivery and/or targeting vehicles comprise a cell receptor-specific ligand and a binding agent. Others comprise a cell receptor-specific ligand to which modfied protein or a polynucleotide encoding a modified protein to be delivered has been operatively attached. For example, several ligands have been used for receptor-mediated gene transfer (Wu and Wu, 1987; Wagner et al., 1990; Perales et al., 1994; Myers, EPO 0273085), which establishes the operability of the technique. In another example, specific delivery in the context of another mammalian cell type has been described (Wu and Wu, 1993; incorporated herein by reference).

In still further embodiments, the specific binding ligand may comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). The asialoglycoprotein, asialofetuin, which contains terminal galactosyl residues, also has been demonstrated to target liposomes to the liver (Spanjer and Scherphof, 1983; Hara et al., 1996). The sugars mannosyl, fucosyl or N-acetyl glucosamine, when coupled to the backbone of a polypeptide, bind the high affinity manose receptor (U.S. Pat. No. 5,432,260, specifically incorporated herein by reference in its entirety). It is contemplated that the cell or tissue-specific transforming constructs of the present invention can be specifically delivered into a target cell or tissue in a similar manner.

In another example, lactosyl ceramide, and peptides that target the LDL receptor related proteins, such as apolipoprotein E3 (“Apo E”) have been useful in targeting liposomes to the liver (Spanjer and Scherphof, 1983; WO 98/0748).

Folate and the folate receptor have also been described as useful for cellular targeting (U.S. Pat. No. 5,871,727). In this example, the vitamin folate is coupled to the complex. The folate receptor has high affinity for its ligand and is overexpressed on the surface of several malignant cell lines, including lung, breast and brain tumors. Anti-folate such as methotrexate may also be used as targeting ligands. Transferrin mediated delivery systems target a wide range of replicating cells that express the transferrin receptor (Gilliland et al., 1980).

C. Liposome/Nucleic Acid Combinations

It is contemplated that when the liposome composition comprises a cell or tissue specific nucleic acid, this technique may have applicability in the present invention. In certain embodiments, lipid-based non-viral formulations provide an alternative to viral gene therapies. Although many cell culture studies have documented lipid-based non-viral gene transfer, systemic gene delivery via lipid-based formulations has been limited. A major limitation of non-viral lipid-based gene delivery is the toxicity of the cationic lipids that comprise the non-viral delivery vehicle. The in vivo toxicity of liposomes partially explains the discrepancy between in vitro and in vivo gene transfer results. Another factor contributing to this contradictory data is the difference in liposome stability in the presence and absence of serum proteins. The interaction between liposomes and serum proteins has a dramatic impact on the stability characteristics of liposomes (Yang and Huang, 1997). Cationic liposomes attract and bind negatively charged serum proteins. Liposomes coated by serum proteins are either dissolved or taken up by macrophages leading to their removal from circulation. Current in vivo liposomal delivery methods use aerosolization, subcutaneous, intradermal, intratumoral, or intracranial injection to avoid the toxicity and stability problems associated with cationic lipids in the circulation. The interaction of liposomes and plasma proteins is largely responsible for the disparity between the efficiency of in vitro (Felgner et al., 1987) and in vivo gene transfer (Zhu et al., 1993; Philip et al., 1993; Solodin et al., 1995; Liu et al., 1995; Thierry et al., 1995; Tsukamoto et al., 1995; Aksentijevich et al., 1996).

An exemplary method for targeting viral particles to cells that lack a single cell-specific marker has been described (U.S. Pat. No. 5,849,718). In this method, for example, antibody A may have specificity for tumor, but also for normal heart and lung tissue, while antibody B has specificity for tumor but also normal liver cells. The use of antibody A or antibody B alone to deliver an anti-proliferative nucleic acid to the tumor would possibly result in unwanted damage to heart and lung or liver cells. However, antibody A and antibody B can be used together for improved cell targeting. Thus, antibody A is coupled to a gene encoding an anti-proliferative nucleic acid and is delivered, via a receptor mediated uptake system, to tumor as well as heart and lung tissue. However, the gene is not transcribed in these cells as they lack a necessary transcription factor. Antibody B is coupled to a universally active gene encoding the transcription factor necessary for the transcription of the anti-proliferative nucleic acid and is delivered to tumor and liver cells. Therefore, in heart and lung cells only the inactive anti-proliferative nucleic acid is delivered, where it is not transcribed, leading to no adverse effects. In liver cells, the gene encoding the transcription factor is delivered and transcribed, but has no effect because no an anti-proliferative nucleic acid gene is present. In tumor cells, however, both genes are delivered and the transcription factor can activate transcription of the anti-proliferative nucleic acid, leading to tumor-specific toxic effects.

The addition of targeting ligands for gene delivery for the treatment of hyperproliferative diseases permits the delivery of genes whose gene products are more toxic than do non-targeted systems. Examples of the more toxic genes that can be delivered includes pro-apoptotic genes such as Bax and Bak plus genes derived from viruses and other pathogens such as the adenoviral E4orf4 and the E.coli purine nucleoside phosphorylase, a so-called “suicide gene” which converts the prodrug 6-methylpurine deoxyriboside to toxic purine 6-methylpurine. Other examples of suicide genes used with prodrug therapy are the E. coli cytosine deaminase gene and the HSV thymidine kinase gene.

It is also possible to utilize untargeted or targeted lipid complexes to generate recombinant or modified viruses in vivo. For example, two or more plasmids could be used to introduce retroviral sequences plus a therapeutic gene into a hyperproliferative cell. Retroviral proteins provided in trans from one of the plasmids would permit packaging of the second, therapeutic gene-carrying plasmid. Transduced cells, therefore, would become a site for production of non-replicative retroviruses carrying the therapeutic gene. These retroviruses would then be capable of infecting nearby cells. The promoter for the therapeutic gene may or may not be inducible or tissue specific.

Similarly, the transferred nucleic acid may represent the DNA for a replication competent or conditionally replicating viral genome, such as an adenoviral genome that lacks all or part of the adenoviral E1a or E2b region or that has one or more tissue-specific or inducible promoters driving transcription from the E1a and/or E1b regions. This replicating or conditional replicating nucleic acid may or may not contain an additional therapeutic gene such as a tumor suppressor gene or anti-oncogene.

d. Lipid Administration

The actual dosage amount of a lipid composition (e.g., a liposome-modified protein or polynucleotide encoding a modified protein) administered to a patient can be determined by physical and physiological factors such as body weight, severity of condition, idiopathy of the patient and on the route of administration. With these considerations in mind, the dosage of a lipid composition for a particular subject and/or course of treatment can readily be determined.

### VI. EXAMPLES

The following examples are included to demonstrate preferred embodiments of the present invention. It should be appreciated by those of skill in the art that that techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar results without departing from the spirit and scope of the invention.

### Example 1

Materials And Methods

Cells and Viruses. Vero and BHK cells were originally obtained from ATCC. HSV-1(F) is the prototype HSV-1 strain used in this laboratory (Ejercito et al., 1967). Recombinant R325 derived from HSV-1(F), includes approximately 800 bp comprising the carboxyl-terminal half of the α4 gene and grows only in a Vero cell line expressing the α4 gene (DeLuca et al., 1985). Both the virus and the cell line were kind gifts of Neal DeLuca (University of Pittsburgh). The U_{S}3 gene was deleted from the recombinant R7041 (Longnecker et al., 1987) and then repaired to yield R7306.

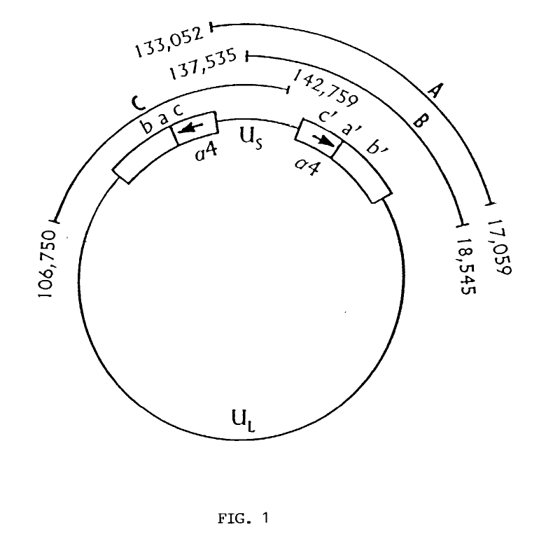

Plasmids and Cosmids. The plasmid pRB5166 was constructed by cloning into the SaII/BamHI sites of the pACYC184 vector a HSV-1 DNA fragment that extends from the Sal I site at +177 with respect to the α4 gene transcription start site and includes the entire BamHI P and BamHI S fragments. The cosmid cloning vector pRB78 was constructed as follows. The multiple cloning site of the Stratagene Supercos1 (La Jolla Calif., cat#251301) was cleaved with EcoRI and replaced with an oligonucleotide(s) containing EcoRI/PacI/Sse8387I/SpeI/BamHI/NdeI/EcoRV/PacI/EcoRI cloning sites. The PacI restriction site, absent in HSV-1, serves to liberates the cloned HSV-1(F) DNA fragments from the vector. HSV-1(F) viral DNA was prepared from virions as previously described. To construct cosmid pBC 1004, which contains the HSV-1(F) sequence n133052 through n17059, viral DNA was partially digested with Sau3A I, dephosphorylated and ligated into the BamHI site of the pRB78 cosmid vector previously linearized with Xba I. The DNA was then packaged using Stratagene Gigapack XLII (La Jolla, Calif.), following the manufacturer's instructions. E. coli L-1 Blue MR was then infected and ampicillin-resistant colonies were screened by restriction enzyme analysis. Insert termini were sequenced to verify mapping. Cosmid pBC1008 was constructed by cloning the BglII F-H fragment (HSV-1(F) n106750 through n142759) into the BamHI site of pRB78 vector. Cosmid pBC1009 was constructed as follows. An NsiI/ScaI DNA fragment was isolated from a double digest of viral DNA. The fragment (HSV-1(F) n137538 through n18545) was ligated into the Sse8387I/EcoRV sites of pBR78. Cosmids PBC1008 and PBC1009 were mapped by restriction enzyme analysis and insert termini were sequenced (FIG. 1).

Electron photomicroscopy. Vero cells infected with HSV-1(F) or d120 were incubated for 20 hrs at 37° C. Cells were fixed in 2% glutaraldehyde in PBS for 60 min at 4° C., post-fixed with 1% osmium tetroxide, en bloc stained with uranyl acetate (5 mg/ml), dehydrated in acetone, and embedded in Epon 812. Thin sections were examined either unstained or poststained with uranyl acetate and lead hydroxide. The cells were photographed at 6000× in a Siemens 101 electron microscope.

Light photomicroscopy. Cells were labeled with biotinylated dUTP. At indicated times the cells were fixed in ice-cold methanol at −20° C. and air-dried, then rinsed in phosphate buffered saline, reacted for 15 min at room temperature with 40 ml of a solution containing 1× terminal transferase buffer (Promega), 1 mM CoCl₂, 0.05 mg/ml bovine serum albumin (BSA), 0.5 nmoles biotin-16-dUTP (Boehringer Mannheim Biochemicals) and 3 units of terminal transferase (TdT, Promega), rinsed extensively in PBS, reacted for 30 min at room temperature with 40 ml of a solution containing Texas red-conjugated avidin in 4×SSC (1×SSC is 0.15 M NaCl, 0.015 M Na-citrate), 0.1% Triton X-100, 5% (w/v) nonfat dry milk, extensively rinsed with PBS, mounted in 10% PBS in glycerol and examined under a Zeiss confocal fluorescence microscope. The images were captured under identical settings with the software provided by Zeiss and printed in a Tektronix 440 phaser printer. Cells were mock infected, 37° C., 20 hrs; infected with d120 virus, 37° C. 20 hrs; infected with HSV-1(F), 37° C., 20 hrs; infected with d120 virus, 37° C. 30 hrs; mock-infected, 39.5° C., 30 hrs.; infected with d120 virus, 39.5° C, 30 hrs.; and infected with HSV-1(F), 39.5° C., 30 hrs.

DNA Fragmentation Assay. Vero cells or E5 cells were infected with HSV-1(F), d120 mutant, or HSV-1 tsHA1 mutant and maintained at 37° C. or 39.5° C. in the absence or in the presence of phosphonoacetic acid. At 30 hrs after infection, 2×106 cells per sample were collected, washed in PBS, lysed in a solution containing 10 mM Tris-HCl, pH 8.0, 10 mM EDTA, and 0.5% Triton X-100, and centrifuged at 12,000 rpm for 25 min in an Eppendorf microcentrifuge to pellet chromosomal DNA. Supernatant fluids were digested with 0.1 mg RNase A per ml at 37° C. for 1 hr, for 2 hrs with 1 mg proteinase K per ml at 50° C. in the presence of 1% sodium dodecylsulphate (SDS), extracted with phenol and chloroform, and precipitated in cold ethanol and subjected to electrophoresis on horizontal 1.5% agarose gels containing 5 mg of ethidium bromide per ml. DNA was visualized by WV light transillumination. Photographs were taken with the aid of a computer-assisted image processor (Eagle Eye II, Stratagene).

Construction of recombinant viruses by marker rescue. Recombinant viruses were constructed using a modification of the technique originally described by Post and Roizman (1981). Vero cells were transfected with plasmid or cosmid DNA using Lipofectamine (Gibco-BRL), according to the manufacturer's instructions. At 6 hr after transfection, the cells were exposed to 0.1 to 1 PFU of HSV-1(Kos)d120 per cell. Recombinant viruses were isolated from single plaques and grown in Vero cells. To obtain HSV-1 120KR, Vero cells were exposed to 10 PFU of HSV-1(KOS)d120 per cell. The infected cells were harvested at 48 hr post infection, frozen-thawn and sonicated, and then serially diluted and titered on Vero cells. Recombinant viruses were isolated from single plaques and grown in Vero cells.

### Example 2

An HSV-1 Mutant Deleted in ICP4 Induces Apoptosis.

In this series of studies, Vero cells infected with wild-type or the d120 mutant were examined for morphologic evidence of apoptosis. Vero cells were fixed and harvested at 20 to 24 hrs after infection with wild-type or d120, embedded, sectioned, and examined in a Siemens 101 electron microscope. The cells infected with wild-type virus showed typical infected cell morphology, i.e., marginated chromatin, separation of inner and outer nuclear membranes, and accumulation of virus particles in some but not all cells. Cells infected with the d120 deletion mutant exhibited extensive condensation of chromatin, obliteration of the nuclear membrane, and extensive vacuolization and blebbing of the cytoplasm. It was estimated that approximately 40 to 50% of the infected cells exhibited some or all of the morphologic changes described above.

In another series of studies, Vero cells were mock-infected or infected with 10 PFU of either the wild-type or the d120 mutant virus per cell. After 20 hrs of incubation at 37° C. the cells were fixed, labeled with biotinylated dUTP in the presence of terminal transferase, and then reacted with fluorescent avidin. Mock-infected cells or cells infected with wild-type virus showed no sign of labeling with biotinylated dUTP by terminal transferase, whereas cells infected with d120 and maintained at the same temperature showed extensive fluorescence due to the reaction of fluorescent avidin to biotinylated dUTP incorporated at the DNA termini created by the cleavage of DNA.

In the third series of studies, replicate Vero cell cultures were infected with 10 PFU of either HSV-1(F) or d120 per cell and incubated at 37° C. The study also included a Vero cell culture infected with HSV-1(F), overlaid with medium containing 300 μg of phosphonoacetate per ml and incubated at 37° C., and a set of Vero cell cultures infected with 10 PFU of HSV-1 tsHA1 and incubated at either 37° C. or 39.5° C. This concentration of phosphonoacetate completely inhibits viral DNA synthesis and blocks the expression of γ₂ genes dependent on viral synthesis for their expression. The cells were harvested at 30 hrs after infection, lysed, and centrifuged to pellet the chromosomal DNA. The supernatant fluids were processed as described above and subjected to electrophoresis in agarose gels to test for the presence of soluble, fragmented DNA.

The results were as follows. Cells infected with d120 deletion mutant yielded high amounts of fragmented DNA which were readily visible on agarose gels stained with ethidium bromide. These ladders were not seen in agarose gels containing electrophoretically separated extracts of wild-type infected cells or E5 cells infected with d120. When Vero cells were incubated in medium containing phosphonoacetate, fragmented DNA was detected from cells infected with d120 mutant but not with the wild-type. Fragmented DNA was visible in extracts of mock-infected cells incubated at 39.5° C., but not in cells infected with HSV-1tsHA1 and incubated at the same temperature.

From this series of studies, it is concluded that (i) HSV-1 is capable of inducing the morphologic and biochemical changes characteristic of apoptosis and these changes are prominent in cells infected with a mutant lacking ICP4; (ii) wild-type virus does not induce apoptosis indicating that ICP4 or a protein expressed subsequently is able to protect cells from apoptosis; (iii) the protective, anti-apoptotic effect is a viral function which does not depend on the onset of viral DNA synthesis; (iv) DNA degradation typical of apoptosis was observed upon incubation at 39.5° C. in mock-infected but not HSV-1tsHA1 infected cells, which suggests that prolonged incubation at the elevated temperature can induce apoptosis that is blocked by a viral function expressed early.

### Example 3

ICP4 Expresses an Anti-Apoptotic Function.

In a fourth series of studies, Vero or E5 cells were mock infected or infected with 10 PFU per cell with either HSV-1(F) or d120. The cells were incubated at 39.5° C. for 30 hrs. The rationale of these studies was as follows. As noted in Example 1, HSV-1(F) carries a ts lesion in the α4 gene and at the nonpermissive temperature (39.5° C.) expresses only α genes. The α4 gene resident in the E5 cell line and the d120 mutant virus lacking the α4 gene were derived from the HSV-1(KOS) strain which does not exhibit the ts phenotype. In addition, the α4 gene resident in the E5 cell line is induced after infection and is not expressed in uninfected cells. In the first series of studies, the cells were harvested, lysed, centrifuged to sediment chromosomal DNA and the supernatant fluids were processed as described in Example 1 and subjected to electrophoresis in agarose gels.

The results were as follows. Fragmented DNA was present in lanes containing electrophoretically separated extracts of mock-infected Vero cells, Vero cells infected with d120 mutant, and the mock infected E5 cells. Fragmented DNA was not detected in Vero cells infected with wild-type virus, or in E5 cells infected with either d120 mutant virus or HSV-1(F) virus.

In the second series of studies Vero cells were mock infected, or infected with either d120 or with wild-type virus. After 30 hrs of incubation at 39.5° C., the cells were fixed and labeled with biotinylated dUTP by terminal transferase, and reacted with fluorescent avidin. Fluorescence was detected in mock-infected or infected with d120 mutant, but not in cells infected with wild-type virus.

These studies permit the conclusion that HSV-1(F) α4 gene encodes a function which blocks apoptosis reflected in the degradation of DNA, and that this function is separable from the repressor and transactivator functions of ICP4 which are affected by the temperature sensitive lesion of the α4 gene of HSV-1(F).

A summary of the results is provided in Table 6. Induction of (+), or protection from (−) apoptosis is indicated upon conditions (infecting virus and incubation temperature) which induce apoptosis in Vero and E5 cell lines. “nt” indicates not tested.

TABLE 6

| | VERO | VERO | E5 | |
|---|---|---|---|---|
| | (37° C.) | (39° C.) | (37° C.) | E5 (39.5° C.) |
| MOCK | − | + | − | + |
| HSV-1(F) | − | − | − | − |
| HSV-1 d120 | + | + | − | − |
| HSV-1 tsHA1 | − | − | nt | − |

### Example 4

HSV-1(KOS)d120 Carries an Additional Mutation.

The results described in this section emerged from studies designed to repair the deletion in both copies of the α4 gene of HSV-1(KOS)d120. The inventors isolated recombinants in which this gene was repaired by two different procedures. In the first, the inventors cloned an HSV-1(F) DNA fragment that contains an α4 sequence plus enough flanking sequence to allow homologous recombination. In this series of studies Vero cells were transfected with plasmid DNA and infected with 0.1 to 1 PFU of HSV-1(KOS)d120 per cell. Under these conditions, plaques formed only in cultures of cells transfected with the plasmid DNA. Recombinant virus was recovered from individual plaques, and designated 120FR (for HSV-1(F) repair).

The second procedure was based on the observation that a small amount of virus recombines with the resident α4 gene in the E5 cell line to yield rescued virus capable of replicating efficiently in the absence of exogenous source of ICP4. The observed recombination frequency is 10⁻⁶ to 10⁻⁷ and therefore such rescued virus would be expected to be present in HSV-1(KOS)d120 stock. To isolate these recombinants, Vero cells were infected at a high multiplicity with HSV-1 d120. At 24 hr after infection cells were harvested, frozen-thawed and serial dilutions were used to infect Vero cells. The recombinant virus obtained in this fashion was designated 120KR (for HSV-1(KOS) repair).

Analyses of DNA extracted from replicate Vero cell cultures infected with HSV-1(KOS)d120, 120FR or 120KR showed ladders typical of apopotic cells. These ladders were absent in extracts of mock-infected cells or cells infected with HSV-1(F). The inventors conclude from these studies that HSV-1(KOS)d120 genome contain an additional mutation other than in the α4 gene.

### Example 5

The U_{S}3 Protein Kinase is Required to Block Apoptosis Induced by HSV-1 Infection.

In the following series of studies, the inventors defined the region that contains the additional mutation in HSV-1(KOS)d120 by rescue of the HSV-1(KOS)d120 with cosmids containing large HSV-1 DNA fragments. The three cosmids used in these studies were cosmid pBC8008, which contains all of the HSV-1 terminal repeat sequence, almost all of the U_{S} sequence, and part of the U_{L} sequence (FIG. 1) and cosmids pBC8004 and pBC8009 which contain fragments spanning over the entire repeat sequence but differ in the extent of coverage of the U_{S} region. Individual plaques purified from each transfection were tested for their ability to protect infected cells from apoptosis induced by infection. The results shown in Table 7 suggested that the second mutation in HSV-1(KOS)d120 may map in the U_{S} domain containing the genes U_{S}1 through U_{S}3.

TABLE 7

| Plasmid/Cosmid | Recombinant | Protection From Apoptosis |
|---|---|---|
| pRB5166 | HSV-1 120FR | 0/9 |
| None | HSV-1 120KR | 0/5 |
| pBC1004 | HSV-1 120AR | 1/4 |
| pBC1009 | HSV-1 120BR | 0/9 |
| pBC1008 | HSV-1 120CR | 8/9 |

To map the function required to block apoptosis more precisely, the inventors took advantage of the availability in this laboratory of several deletion mutants which span the sequence thought to encode the gene required to block apoptosis in infected cells. Recombinant virus R325 lacks the carboxyl terminal half of the α22 gene, and the 3′ domain of the U_{S}2 gene. Recombinant virus R7041, lacks most of the U_{S}3 gene whereas in recombinant R7306 the U_{S}3 gene had been repaired. The results indicate that apoptosis was induced in cells infected with R7041 (ΔU_{S}3) but not in cells infected with the other mutants.

Two series of studies verified the conclusion that a functional U_{S}3 gene is required for prevention of apoptosis and that the second mutation in the HSV-1(KOS)d120 resides in the U_{S}3 gene. In the first series, the inventors carried out simple complementation tests to determine whether the second mutation in HSV-1(KOS)d120 was the same as that in the rescued virus or in R7041 (ΔU_{S}3) recombinant. Specifically Vero cells were infected with artificial mixtures of 120FR and HSV-1(KOS)d120, 120FR and R7041, or 120FR and R7306 (U_{S}3 repaired). The results indicate the following. As could be expected, 120FR did not complement HSV-1(KOS)d120, the parent virus from which it was derived. The second, mixture of 120FR and R7041 also did not complement each other suggesting that 120FR and its parent virus, HSV-1(KOS)d120 contained a nonfunctional U_{S}3. Verification of this hypothesis emerged from the observation that R7306 containing a repaired U_{S}3 gene complemented 120FR and blocked apoptosis in cells infected with these two viruses.

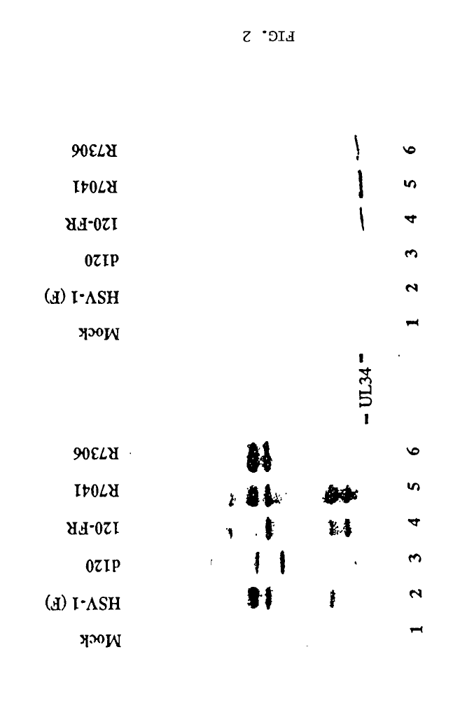

The second series of studies served to verify the HSV-1(KOS)d120 and its derivatives lacked a functional U_{S}3 gene. As described in this introduction, the production of U_{L}34 gene has been shown in earlier studies from this laboratory to be a substrate for the U_{S}3 protein kinase. In wild-type infected cells, U_{L}34 has an apparent Mᵣ of 30,000 whereas in cells infected with R7041 (ΔU_{S}3), U_{L}34 has an apparent migrates of Mᵣ 33,000. Inasmuch as the original studies were done in BHK cells, in this series of studies replicate cultures of BHK cells were infected 10 PFU of HSV-1(F), 120FR, R7041, and R7306 per cell. The results, shown in FIG. 2 indicate that the fully processed U_{L}34 protein was present in cells infected with HSV-1(F) but not cells infected with 120FR or R7041. As could be expected, the cells infected with HSV-1(KOS)d120 did not make detectable quantities of U_{L}34 protein inasmuch as they lack the α4 gene. In the absense of α4 gene expression there is no β or γ gene expression and therefore, no U_{S}3 gene expression. Taken together, the inventors' results indicate the U_{S}3 kinase is functionally absent in HSV-1(KOS)d120 and 120FR. The inventors conclude that U_{S}3 is required for protection from apoptosis induced by HSV-1.

### Example 6

Materials and Methods

Cells and viruses. Rabbit skin cells were originally obtained by J. McLaren and grown in Dulbecco's modified Eagle medium (DMEM) supplemented with 5% newborn calf serum.

Plasmids. An EcoRI/NotI fragment from pEBG-mBAD (Cell Signalling, Beverly, Mass.) containing the mouse BAD open reading fused to glutathione S transferase (GST) was cloned into the baculovirus transfer vector MTS-1. MTS-1 contains a CMV promoter inserted into the XhoI/EcoRI sites of pAcSG2 (Pharmingen, San Diego, Calif.). An EcoRI/BamHI fragment encoding GST was removed creating BAD-MTS-1 that contains the BAD open reading frame under the control of the CMV promoter.

Baculoviruses. A BAD expressing baculovirus was constructed by cotransfecting the BAD-MTS-1 transfer plasmid, with Baculogold DNA (Pharmingen, San Diego, Calif.) as per manufacturer's instructions. Efficient baculovirus gene expression in mammalian cells requires treatment with sodium butyrate, a histone deacetylase inhibitor (Meigner et al., 1988). In all experiments in which rabbit skin cells were infected with baculoviruses, all infected or treated cultures were exposed to medium containing 5 mM sodium butyrate after 2 hrs of viral infection at 37° C.

Immunoblot assays. Cells were harvested by scrapping cells into their medium, pelleted by low speed centrifugation, rinsed twice with PBS A (0.14M NaCl, 3 mM KCL, 10 mM Na₂HPO₄, 1.5 mM KH₂PO₄), lysed in RIPA buffer (1% NP-40, 0.5% sodium deoxycholate, and 0.1% SDS in PBS A), stored on wet ice for 10 min before centrifugation at 14,000×g for 10 min. The protein concentration of the supernatant fluids was determined with the aid of the Bio-Rad protein assay (Bio-Rad Laboratories, Hercules, Calif.) according to directions provided by the manufacturer. Protein samples, denatured in disruption buffer (50 mM tris pH 7.0, 2.75% sucrose, 5% β-ME, 2% SDS) were electrophoretically separated in a 12% denaturing polyacrylamide gel (100 μg of protein per lane), electrically transferred to a nitrocellulose sheet, blocked, and reacted with a rabbit polyclonal antibody specific for either poly(ADP-ribose) polymerase (PARP, Santa Cruz Biotechnologies, Santa Cruz, Calif.), BAD, BAD-ser136P, BAD-ser155P (Cell Signalling Technology, Beverly, Mass.), or mouse monoclonal specific for BAD-ser112P. Protein bands were visualized with either alkaline phosphatase or through enhanced chemiluminescent detection (ECL) according to the instructions of the manufacturer (Amersham, Buckinghamshire, England).

Measurement of DEVDase activity. Cellular extracts were assayed for caspase-3 activity with a tetrapeptide (Asp-Glu-Val-Asp) conjugated to phenylnitraniline (DEVD-pNA). Rabbit skin cells grown in 25 cm² flask cultures were either mock infected or infected with recombinant baculoviruses as indicated. At 18 hrs after infection, the cells were scraped in their medium, rinsed twice with PBS A, resuspended in 150 μl lysis buffer (50 mM HEPES, pH 7.4, 0.1% CHAPS, 1 mM DTT, 0.1 mM EDTA) and left on ice for 5 min. The lysates were then centrifuged at 14,000×g for 10 min at 4° C. and the supernatant was collected and tested for DEVDase activity as recommended by the manufacturer (BIOMOL). The released chromophore was measured in a spectrophotometer at 405 nm after 2 hrs.

DNA fragmentation assay. Infected or uninfected cells were collected, washed in PBS, lysed in a solution containing 10 mM Tris-HCl (pH 8.0), 10 mM EDTA, and 0.5% Triton X-100, centrifuged at 14,000 rpm for 25 min in an Eppendorf microcentrifuge to pellet chromosomal DNA. The supernatant was digested with RNase A (0.1 mg/ml) at 37° C. for 1 hr, and digested with proteinase K (1 mg/ml) at 50° C. for 2 hrs in the presence of 1% sodium dodecyl sulfate, precipitated in cold ethanol, and subjected to electrophoresis in 1.5% agarose gels containing 0.5 mg of ethidium bromide per ml. Oligonucleosomal DNA fragments were visualized by UV light transillumination. Photographs were taken with the aid of Eagle Eye II (Stratagene).

### Example 7

Induction of Apoptosis by a Baculovirus Expressing the BAD Protein.

To facilitate the studies on the role of the U_{S}3 protein kinase in blocking apoptosis, it became critical to express the BAD protein to all cells in a dose dependent manner. BAD was delivered to cells by exposing cells to a recombinant baculovirus designated Bac-BAD, which expresses the BAD open reading frame driven by the hCMV immediate early promoter as described in Example 6. Rabbit skin cells were either mock infected, or exposed to 5 PFU of Bac-WT or Bac-BAD per cell. The cells were harvested at 18 hrs after addition of Bac-BAD, solubilized, electrophoretically separated in a denaturing polyacrylamide gel, transferred to a nitrocellulose sheet and reacted with the anti-BAD antibody. The BAD protein was detected in cells infected with Bac-BAD but not in mock or Bac-WT infected cells. None of the experiments described in this study were able to detect eodogenous BAD. In addition, a potential BAD cleavage product of M_{R} 15,000 was also detected.

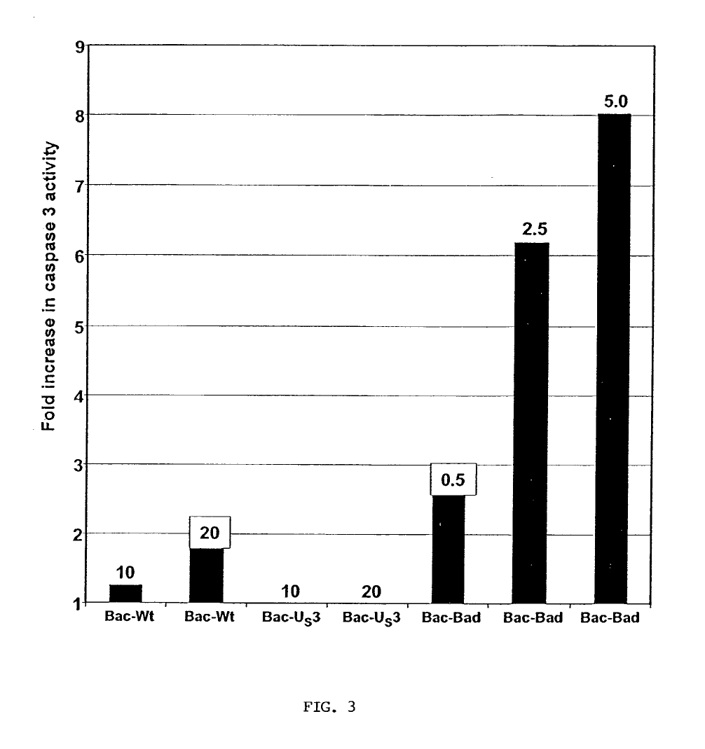

To address whether baculovirus mediated overexpression of BAD is sufficient to induce apoptosis, rabbit skin cells were mock-infected or exposed to 10 or 20 PFU of Bac-WT or Bac-U_{S}3, or to 0.5, 2.5 or 5.0 PFU of Bac-BAD per cell. Cells were harvested at 18 hrs after infection, lysed and tested for DEVDase activity. The results, normalized with respect to the level of caspase activity in mock-infected cells are shown in FIG. 3 and were as follows: Cells infected with either 10 or 20 PFU of Bac-WT per cell exhibited less than a two-fold increase in DEVDase activity. In cells infected with either 10 or 20 PFU of Bac-U_{S}3 per cell, the DEVDase activity was identical to that of mock infected cells. The DEVDase activity of cells infected with 0.5, 2.5 or 5.0 PFU of Bac-BAD per cell exhibited a 2.5-, 6- or 8-fold increase in DEVDase activity, respectively, relative to that of mock-infected cells. These results indicated that while Bac-WT and Bac-U_{S}3 did not induce significant amounts of caspase activity, Bac-BAD induced significant caspase activity in a dose dependent manner.

### Example 8

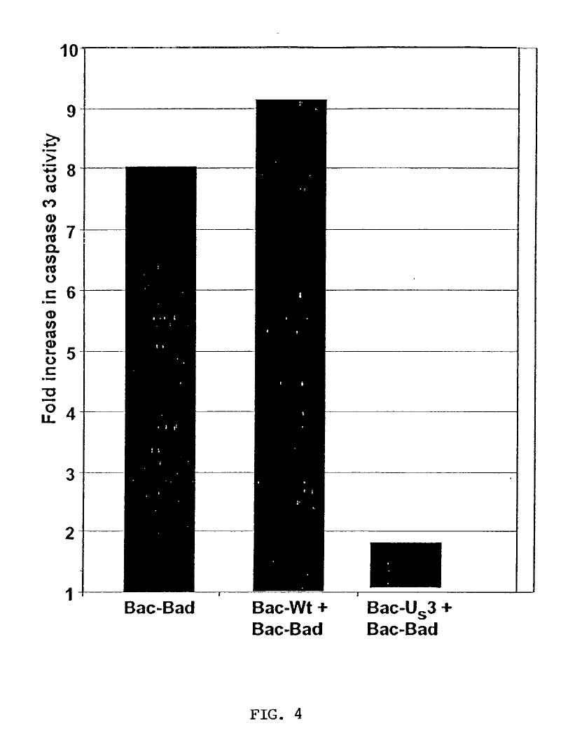

U_{S}3 Protein Kinase Blocks DEVDase Activity Induced by BAD Expression The objective of this series of experiments was to test whether the U_{S}3 protein kinase is sufficient to prevent BAD-induced apoptosis. Rabbit skin cells were either mock infected, or infected with 20 PFU of Bac-WT or Bac-U_{S}3 per cell 6.5 hrs prior to exposure of the cells to 5.0 PFU of Bad-BAD per cell. The cells were harvested at 18 hrs after Bac-BAD infection and tested for DEVDase activity. As shown in FIG. 4, rabbit skin cells either mock infected or infected with Bac-WT prior prior to exposure to Bac-BAD, exhibited a 8- and 9-fold increase in DEVDase activity, respectively, relative to that of mock-infected cells. In contrast, cells infected with Bac-U_{S}3 prior to Bac-BAD addition exhibited significantly less DEVDase activity (less than 2 fold increase). These results indicate that U_{S}3 is sufficient to prevent BAD-induced caspase activation.

### Example 9

U_{S}3 Protein Kinase Blocks the Cleavage of poly(ADP-ribose)polymerase (PARP)-Induced by BAD Expression

To further verify that Bac-BAD induced caspase activity and that Bac-U_{S}3 blocked Bac-BAD-induced caspase activity, the status of PARP, a substrate of caspase 3, was examined. Active caspase 3 cleaves the Mᵣ 110,000 PARP to yield a truncated polypeptide with an Mᵣ of 85,000. To address whether U_{S}3 could prevent BAD induced PARP cleavage, rabbit skin cells were either mock infected, or exposed to 20 PFU of Bac-WT or Bac-U_{S}3 per cell 6.5 hrs prior to exposure to 5 PFU of Bac-Bad per cell. The cells were harvested at 18 hrs after addition of Bac-Bad, solubilized, electrophoretically separated in a denaturing polyacrylamide gel, transferred to a nitrocellulose sheet and reacted with the anti PARP antibody. The results were as follows: The Mᵣ 85,000 PARP cleavage product was not detected in mock, Bac-WT, or Bac-U_{S}3 infected cells that had not been treated with Bac-Bad. The Mᵣ 85,000 PARP cleavage product was readily detected in mock- or in Bac-WT-infected cells that had been exposed to Bac-Bad, but not in Bac-U_{S}3 infected cells that had been exposed to Bac-Bad. Thus, the U_{S}3 protein kinase prevented the cleavage of PARP by caspases induced by BAD.

### Example 10

U_{S}3 Protein Kinase Blocks DNA Fragmentation Induced by BAD Expression

The objective of this series of experiments was to further verify that U_{S}3 blocks BAD induced apoptosis by testing whether U_{S}3 prevents the fragmentation of cellular DNA induced by BAD. Rabbit skin cells were either mock infected, or infected with 20 PFU of Bac-WT or Bac-U_{S}3 per cell 6.5 hrs prior to exposure to 5 PFU Bac-Bad per cell. The cells were harvested 18 hrs after exposure to Bac-Bad and processed as described in Example 6. Cellular DNA was fragmented in cells mock-infected or infected with Bac-WT prior to exposure to Bac-Bad, but not in mock-infected cells or cells infected with Bac-WT or Bac-U_{S}3. In contrast, DNA fragmentation was not observed in extracts of Bac-Bad treated cells that had been previously exposed to Bac-U_{S}3. U_{S}3 protein kinase is sufficient to block BAD induced apoptosis.

### Example 11

Bac-U_{S}3 Mediates Posttranslational Modification of BAD

BAD phosphorylated at serines 112 and 136 is sequestered by the 14-3-3 proteins. In turn this step blocks the translocation of BAD to the mitochondria and its pro-apoptotic activity (Harada et al., 1999; Pastorino et al., 1998; Zha et al., 1996; Hsu et al., 1997). In addition, phosphorylation of BAD at serine 155 appears to be necessary in order to release the protein from its association with Bc1-2 family member Bc1-X_{L} (30-32). It was of interest to determine whether U_{S}3 prevents BAD-induced apoptosis by posttranslational modification of BAD. To resolve this question, rabbit skin cells were mock infected or infected with 20 PFU of Bac-WT or Bac-U_{S}3 per cell 6.5 hrs prior to the exposure of the cells to Bac-Bad (5 PFU/cell). The cells were harvested 18 hrs after exposure to Bac-BAD, solubilized, electrophoretically separated in denaturing polyacrylamide gels, transferred to a nitrocellulose sheet and reacted with antibody specific for total BAD protein or to either BAD-ser136P, BAD-155P, or BAD-ser112P. The results were as follows:

(i) The total amount of BAD protein detected by anti-BAD antibody in lysates of cells infected with Bac-U_{S}3 prior to exposure to Bac-BAD migrated more slowly and was significantly reduced in amount relative to those detected in lysates of cells exposed to BAC-WT or mock-infected prior to exposure to Bac-BAD. In this series of experiments we detected BAD only in cells exposed to Bac-BAD. It is of note that Bac-U_{S}3 did not affect the accumulation of an irrelevant viral protein expressed by a recombinant baculovirus. These results indicate that U_{S}3 mediates both a posttranslational processing of BAD and a reduction in the amount of total BAD protein.

(ii) The levels of BAD phosphorylated at ser136, ser155, and ser112 present in cells mock infected were similar to those present in cells infected with Bac-WT or Bac-U_{S}3. Inasmuch as the total amount of BAD was significantly reduced in cells exposed to Bac-U_{S}3, this observation suggests that in cells treated with Bac-U_{S}3 the total amount of Bad protein decreased, the amount of BAD protein phosphorylated at specific serines remained the same as in cells exposed to Bac-Bad in the absence of Bac-U_{S}3. The results suggest therefore that U_{S}3 down regulated primarily, if not exclusively, active, pro-apoptotic, nonphosphorylated BAD and concomitantly mediated posttranslational modification of all phosphorylated forms of the protein.

(iii) BAD cleavage products were present in cells that had been mock infected or Bac-WT infected prior to Bac-BAD addition, but were absent in cells infected with Bac-U_{S}3 prior to Bac-Bad addition. Recently it has been reported that caspase mediated cleavage of BAD results in a M_{R} 15,000 truncated product that stimulates BAD's apoptotic activity (35). That Bac-U_{S}3 prevents the cleavage of BAD and is consistent with the evidence that U_{S}3 protein kinase prevents BAD from inducing apoptosis.

These results indicate that U_{S}3: (i) mediated a posttranslational modification of full length Bad, (ii) mediated a decrease in the total amount of BAD, and (iii) prevented the accumulation of a Mᵣ15,000 cleavage product of BAD, all of which could serve to block the pro-apoptotic activity of BAD.

All of the compositions and methods disclosed and claimed herein can be made and executed without undue experimentation in light of the present disclosure. While the compositions and methods of this invention have been described in terms of embodiments, it will be apparent to those of skill in the art that variations may be applied to the compositions and methods, and in the steps or in the sequence of steps of the methods, described herein without departing from the concept, spirit, and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VI. REFERENCES

The following references, to the extent that they provide exemplary procedural details or other information supplementary to that set forth herein, are incorporated by reference:

Pending application Ser. No. 07/931,811

U.S. Pat. No. 4,162,282

U.S. Pat. No. 4,196,265

U.S. Pat. No. 4,310,505

U.S. Pat. No. 4,533,254

U.S. Pat. No. 4,554,101

U.S. Pat. No. 4,659,774

U.S. Pat. No. 4,676,980

U.S. Pat. No. 4,682,195

U.S. Pat. No. 4,683,202

U.S. Pat. No. 4,684,611

U.S. Pat. No. 4,728,575

U.S. Pat. No. 4,728,578

U.S. Pat. No. 4,737,323

U.S. Pat. No. 4,797,368

U.S. Pat. No. 4,816,571

U.S. Pat. No. 4,879,236

U.S. Pat. No. 4,921,706

U.S. Pat. No. 4,952,500

U.S. Pat. No. 4,959,463

U.S. Pat. No. 5,028,592

U.S. Pat. No. 5,139,941

U.S. Pat. No. 5,141,813

U.S. Pat. No. 5,214,136

U.S. Pat. No. 5,223,618

U.S. Pat. No. 5,264,566

U.S. Pat. No. 5,302,523

U.S. Pat. No. 5,322,783

U.S. Pat. No. 5,354,855

U.S. Pat. No. 5,359,046

U.S. Pat. No. 5,378,825

U.S. Pat. No. 5,384,253

U.S. Pat. No. 5,401,511

U.S. Pat. No. 5,428,148

U.S. Pat. No. 5,432,260

U.S. Pat. No. 5,446,137

U.S. Pat. No. 5,464,765

U.S. Pat. No. 5,466,786

U.S. Pat. No. 5,470,967

U.S. Pat. No. 5,538,877

U.S. Pat. No. 5,538,880

U.S. Pat. No. 5,539,082

U.S. Pat. No. 5,550,318

U.S. Pat. No. 5,554,744

U.S. Pat. No. 5,563,055

U.S. Pat. No. 5,563,055

U.S. Pat. No. 5,574,146

U.S. Pat. No. 5,580,859

U.S. Pat. No. 5,589,221

U.S. Pat. No. 5,589,466

U.S. Pat. No. 5,591,616

U.S. Pat. No. 5,602,240

U.S. Pat. No. 5,602,244

U.S. Pat. No. 5,603,872

U.S. Pat. No. 5,610,042

U.S. Pat. No. 5,610,289

U.S. Pat. No. 5,614,617

U.S. Pat. No. 5,623,070

U.S. Pat. No. 5,645,897

U.S. Pat. No. 5,652,099

U.S. Pat. No. 5,656,610

U.S. Pat. No. 5,670,663

U.S. Pat. No. 5,672,697

U.S. Pat. No. 5,681,947

U.S. Pat. No. 5,693,762

U.S. Pat. No. 5,700,922

U.S. Pat. No. 5,702,932

U.S. Pat. No. 5,705,629

U.S. Pat. No. 5,708,154

U.S. Pat. No. 5,714,331

U.S. Pat. No. 5,714,331

U.S. Pat. No. 5,714,606

U.S. Pat. No. 5,719,262

U.S. Pat. No. 5,719,262

U.S. Pat. No. 5,736,336

U.S. Pat. No. 5,736,524

U.S. Pat. No. 5,763,167

U.S. Pat. No. 5,766,855

U.S. Pat. No. 5,766,855

U.S. Pat. No. 5,773,571

U.S. Pat. No. 5,777,092

U.S. Pat. No. 5,780,448

U.S. Pat. No. 5,786,214

U.S. Pat. No. 5,786,461

U.S. Pat. No. 5,789,215

U.S. Pat. No. 5,792,847

U.S. Pat. No. 5,849,718

U.S. Pat. No. 5,858,988

U.S. Pat. No. 5,861,155

U.S. Pat. No. 5,871,727

U.S. Pat. No. 5,871,986

U.S. Pat. No. 5,872,232

U.S. Pat. No. 5,879,703

U.S. Pat. No. 5,886,165

U.S. Pat. No. 5,891,625

U.S. Pat. No. 5,891,625

U.S. Pat. No. 5,908,845

U.S. Pat. No. 5,939,277

U.S. Pat. No. 5,945,100

U.S. Pat. No. 5,965,703

U.S. Pat. No. 5,981,274

U.S. Pat. No. 5,994,136

U.S. Pat. No. 5,994,624

U.S. Pat. No. 6,013,516

U.S. Pat. No. 6,020,192

U.S. Pat. No. 6,054,297

U.S. Pat. No. 6,099,842

U.S. Pat. No. 6,107,090

UK Patent Application GB 2193095

EP 01219

EP 03089

EP 266032

EP 0273085

PCT/US85/01161

PCT/US89/05040

WO 91/00360

WO 92/20702

WO 92/200373

WO 93/08829

WO 94/09699

WO 95/06128

WO 98/0748

WO 99/18933

Aksentijevich et al., Hum. Gene Ther., 7(9):1111-22, 1996.

Asano et al., J. Gen. Virol. 80:51-56, 1999.

Asano et al., Neurosci. Lett. 294:105-8, 2000.

Auber et al., J. Virology, 73:2803-2813, 1999.

Aubert et al., J. Virology, 73:10359-70, 1999.

Ausubel, In: Current protocols in molecular biology, NY, John Wiley and Sons, 1996.

Baichwal and Sugden, In: Gene Transfer, Kucherlapati, (ed.), NY, Plenum Press, 117-148, 1986.

Bangham, et al., J. Mol. Biol., 13:238-252, 1965.

Batterson et al., J. Virology, 45:397-407, 1983.

Batterson et al., J. Virology, 46:371-377, 1983.

Bedzyk et al., J. Biol. Chem., 265:18615-20, 1990.

Benvenisty et al., Proc. Natl. Acad. Sci. USA, 83:9551-9555, 1986.

Berzal-Herranz, et al., Genes and Devel., 6:129-134, 1992.

Bittner et al., Methods in Enzymol., 153:516-544, 1987.

Blomer et al., J. Virology, 71(9):6641-6649, 1997.

Brutlag et al., CABIOS, 6:237-245, 1990.

Campbell et al., J. Mol. Biol., 180:1-19, 1984.

Canfield et al., Methods in Enzymology, 189, 418-422, 1990.

Cech et al., Cell, 27:487-496, 1981.

Chaudhary et al., Proc. Natl. Acad. Sci. USA, 87:9491-9494, 1990.

Chen et al., Mol. Cell Biol., 7:2745-2752, 1987.

Cheng, et al., Invest. Radiol, 22:47-55, 1987.

Cheresh et al., Cancer Res., 46:5112-5118, 1986.

Chittenden et al., EMBO J., 14:5589-5596, 1995.

Chou et al., Adv. Enzymol. Relat. Areas Mol. Biol., 47:45-148, 1978a.

Chou et al., Ann. Rev. Biochem. 47:251-276, 1978b.

Chou et al., Biochemistry, 13(2):222-245, 1974a.

Chou et al., Biochemistry, 13(2):211-222, 1974b.

Chou et al., Biophys. J., 26:367-384, 1979.

Chou et al., Proc. Natl. Acad. Sci. USA, 89:3266-3270, 1992.

Chowrira, et al., Biochem., 32:1088-1095, 1993.

Chowrira, et al., J. Biol. Chem., 269:25856-25864, 1994.

Clem et al., In: Apoptosis II. The Molecular Basis of Apoptosis in Disease, Tomei and Cope (eds.), Cold Spring Harbor Laboratory Press, 89-110, 1994.

Condorelli et al., Mol. Cell Biol., 21:3025-36, 2001.

Condreay et al., Proc. Natl. Acad. Sci. USA, 96:127-132, 1999.

Conley et al., J. Virology, 37:191-206, 1981.

Cotten et al., Proc. Natl. Acad. Sci. USA, 89(13):6094-6098, 1992.

Coupar et al., Gene, 68:1-10, 1988.

Curiel et al., Nat. Immun., 13(2-3):141-164, 1994.

Datta et al., Mol. Cell., 6:41-51, 2000.

Deamer et al., In: Liposomes, Ostro (ed.), Marcel Dekker, Inc., NY, 27-52, 1983.

De Jager, et al., Semin. Nucl. Med., 23(2):165-79, 1993.

del Peso et al., Science, 278:687-689, 1997.

DeLuca et al., J. Virology, 56:558-570, 1985.

Dixon et al., J. Virology, 36:189-203, 1980.

Doolittle, et al., Methods Mol. Biol., 109:215-237, 1999.

Dubensky et al., Proc. Natl. Acad. Sci. USA, 81:7529-7533, 1984.

Egholm et al., Nature, 365:566, 1993

Ejercito et al., J. Gen. Virol., 2:357-364, 1968.

el Gorab, et al., Biochim. Biophys. Acta, 306(1):58-66, 1973.

Faber et al., Nucleic Acids Res., 14:6067-6083, 1986.

Fechheimer et al., Proc. Natl. Acad. Sci. USA, 84:8463-8467, 1987.

Felgner et al., Proc Natl Acad Sci USA, 84(21):7413-7417, 1987.

Fendler et al., In: Catalysis in Micellar and Macromolecular Systems, Academic Press, NY, 1975.

Ferkol et al., FASEB J., 7:1081-1091, 1993.

Fetrow et al., Biotechnology, 11:479-483, 1993.

Forster and Symons, Cell, 49:211-220, 1987.

Fraley et al., Proc. Natl. Acad. Sci. USA, 76:3348-3352, 1979.

Franke et al., Cell, 91:231-241, 1997.

Freshner, In: Animal Cell Culture: A Practical Approach, Second Edition, Oxford/NY, IRL Press, Oxford University Press, 1992.

Friedmann, Science, 244:1275-1281, 1989.

Froehler et al., Nucl. Acids Res., 14:5399-5407, 1986.

Gabizon et al., Cancer Res., 50(19):6371-8, 1990.

Galvan et al., J. Virology, 73:3219-3226, 1999.

Galvan et al., J. Virology, 74:1931-1938, 2000.

Galvan et al., Proc. Natl. Acad. Sci. USA, 95:3931-3936, 1998.

Gerlach et al., Nature (London), 328:802-805, 1987.

Ghosh et al., In: Liver diseases, targeted diagnosis and therapy using specific receptors and ligands, NY, Marcel Dekker, 87-104, 1991.

Gilliland et al., Cancer Res., 40(10):3564-9, 1980.

Glorioso et al., Ann. Rev. Microbiol., 49:675-710, 1995.

Gopal, Mol. Cell. Biol., 5:1.188-1190, 1985.

Gorczyca et al., Cancer Res., 53:1945-1951, 1993.

Graham et al., J. Virology, 52:456-467, 1973.

Gregoriadis, et al., Biochem. Biophys. Res. Commun., 89(4):1287-1293, 1979.

Grunhaus, et al., Seminar in Virology, 3:237-252, 1992.

Gulbis, et al., Hum. Pathol., 24(12):1271-1285, 1993.

Hara et al., Biochim. Biophys. Acta, 1278:51-58, 1996.

Harada et al., Mol. Cell., 3:413-422, 1999.

Harland et al., J. Cell. Biol., 101:1094-1099, 1985.

Harlow and Lane, In: Antibodies: A Laboratory Manual, Cold Spring Harbor Laboratory, 1988.

Haseloff et al. Nature, 334(6183):585-591, 1988.

Hata et al., Microbes. Infect., 1:601-607, 1999.

Heath, et al., Chem. Phys. Lipids, 40:347, 1986.

Henderson et al., Proc. Natl. Acad. Sci. USA, 90:8479-8483, 1993.

Herz et al., Cell, 33:145-151, 1983.

Holland et al., J. Virology, 101: 10-18, 1980.

Honess et al., J. Virology, 14:8-19, 1974.

Honess et al., J. Virology, 16:1308-1326, 1975.

Hope et al., Biochimica. et Biophysica. Acta, 812: 55-65, 1985.

Horwich et al. J. Virology, 64:642-650, 1990.

Hsu et al., Mol. Endocrinol., 12:1858-1867, 1997.

Jameson et al., Comput. Appl. Biosci., 4(1):181-186, 1988.

Jerome et al., J. Virology, 73:8950-8957, 1999.

Johnson et al., In: Biotechnology and Pharmacy, Pezzuto et al (eds.), Chapman and Hall, NY, 1993.

Joyce, Nature, 338:217-244, 1989.

Kaeppler et al., Plant Cell Reports, 9:415-418, 1990.

Kaneda et al., Science, 243:375-378, 1989.

Kato et al., J. Biol. Chem., 266:3361-3364, 1991.

Kelleher et al., Biotechniques, 17(6):1110-1117, 1994.

Kim and Cech, Proc. Natl. Acad. Sci. USA, 84:8788-8792, 1987.

Klein et al., Nature, 327:70-73, 1987.

Komberg and Baker, In: DNA Replication, 2nd Ed., Freeman, San Francisco, 1992.

Koyama et al., J. Virology, 71:2567-2571, 1997.

Kristie et al., Proc. Natl. Acad. Sci. USA, 83:3218-3222, 1986a.

Kristie et al., Proc. Natl. Acad. Sci. USA, 83:4700-4704, 1986b.

Kuddus et al., J. Virology, 69:5568-5575, 1995.

Kyte et al., J. Mol. Biol., 157(1):105-132, 1982.

Laughlin et al, J. Virology, 60:515-524, 1986.

Leader et al., Biochim. Biophys. Acta, 1091:426-431, 1991.

Lebkowski, et al., Mol. Cell. Biol., 8:3988-3996, 1988.

Lemaster et al., J. Virology, 35:798-811, 1980.

Leopardi et al., J. Virology, 71(2):1133-9, 1997.

Leopardi et al., J. Virology, 69:3042-3048, 1995.

Leopardi et al., Proc. Natl. Acad. Sci. USA, 93:9583-9587, 1996.

Leopardi et al., Proc. Natl. Acad. Sci. USA,94:7891-7896, 1997.

Leopardi et al., Proc. Natl. Acad. Sci. USA, 93:9583-9587.

Lieber and Strauss, Mol. Cell. Biol., 15: 540-551, 1995.

Liu et al., Biochim. Biophys. Acta, 1240(2):277-284, 1995.

Longnecker et al., Science, 236:573-576, 1987.

Mann et al., Cell, 33:153-159, 1983.

Martin et al., Nature, 345(6277):739-743, 1990.

Mayer et al., Biochimica. et Biophysica. Acta, 858:161-168, 1986.

Mayhew et al., Biochimica. et Biophysica. Acta, 775:169-174, 1984.

Mayhew et al., Methods Enzymol., 149:64-77, 1987.

McCarthy et al., Proc. Natl. Acad. Sci. USA, 91:3979-3983, 1994.

McLaughlin et al., J. J. Virology, 62:1963-1973, 1988.

Meignier et al., J. Virology, 162:251-254, 1988.

Michael et al., Proc. Natl. Acad. Sci. USA, 86:9808-9812, 1989.

Michael et al., Proc. Natl. Acad. Sci. USA, 90:2286-2290, 1993.

Michael et al., Science, 239:1531-1534, 1988.

Michel and Westhof, J. Mol. Biol., 216:585-610, 1990.

Miller et al., Am. J. Clin. Oncol., 15(3):216-21, 1992.

Millstein and Cuello, Nature, 305:537-539, 1983.

Mizrahi, Process Biochem., 9-12, 1983.

Montaldo, et al., Int. J. Cancer, 81:262-267, 1999.

Muller, J. J. Virology, 61:858-865, 1987.

Munger et al., J. Virology, 75:5491-5497, 2001.

Muzyczka, Curr. Top. Microbiol. Immunol., 158:97-129, 1992.

Nabel et al., Science, 244(4910):1342-4, 1989.

Naldini et al., Proc. Natl. Acad. Sci. USA, 93(21):11382-11388, 1996.

Nicolas et al., In: Vectors: A survey of molecular cloning vectors and their uses, Rodriguez and Denhardt (eds.), Stoneham: Butterworth, 494-513, 1988.

Nicolau et al., Biochem. Biophys. Acta, 721:185-190, 1982.

Nicolau et al., Methods Enzymol., 149:157-176, 1987.

Oltvai et al., Cell, 74:609-619, 1993.

Omirulleh et al., Plant Mol. Biol., 21:415-28, 1993.

Oz et al., J. Vasc. Surg., 11(5):718-725, 1990.

Pagnan et al., Int. J. Cancer, 81:268-74, 1999.

Palukaitis et al., J. Virology, 99:145-151, 1979.

Pan et al., Genes Dev., 8:1285-1299, 1994.

Paskind et al., J. Virology, 67:242-248, 1975.

Pastorino et al., J. Biol. Chem., 274:19411-19416, 1998.

Perales et al., Proc. Natl. Acad. Sci. USA, 91:4086-4090, 1994.

Perriman. et al., Gene, 113:157-163, 1992.

Perrotta and Been, Biochemistry, 31:16, 1992.

Philip et al., J. Biol. Chem., 268:16087-90, 1993.

Phillips et al., In: Large Scale Mammalian Cell Culture, Feder and Tolbert (eds.), Academic Press, Orlando, Fla., 1985.

Post et al., Cell, 24:555-565, 1981.

Post et al., Cell, 25:227-232, 1981.

Potrykus et al., Mol. Gen. Genet., 199:183-188, 1985.

Potter et al., Proc. Natl. Acad. Sci. USA, 81:7161-7165, 1984.

Prody, et al., Science, 231, 1577-1580, 1986.

Purves et al., Biochim. Biophys. Acta, 889:208-215, 1986.

Purves et al., J. Virology, 61:2896-2901, 1987.

Purves et al., J. Virology, 4295-4303, 1992.

Purves et al., J. Virology, 61:2896-2901, 1987.

Purves et al., J. Virology, 65:5757-5764, 1991.

Rao et al. Proc. Natl. Acad. Sci. USA, 89:7742-7746, 1992.

Ray et al., Cell, 69:597-604, 1992.

Reinhold-Hurek and Shub, Nature, 357:173-176, 1992.

Ridgeway et al., In: Vectors: A survey of molecular cloning vectors and their uses, Stoneham: Butterworth, 467-492, 1988.

Rippe et al., Mol. Cell Biol., 10:689-695, 1990.

Roizman et al., In Fields' Virology, 3rd Edition, Fields, et al. (eds.), Raven Press, NY, 2231-2295, 1996.

Roux et al., Proc. Natl. Acad. Sci. USA, 86:9079-9083, 1989.

Salomoni et al., Blood, 96:676-684., 2000.

Sambrook et al., In: Molecular Cloning: A Laboratory Manual, Cold Spring Harbor Laboratory Press, Cold Spring Harbor, N.Y., 1(7):19-17.29, 1989.

Sambrook et al., Molecular Cloning: A Laboratory Manual, 3rd Ed., Cold Spring Harbor Press, Cold Spring Harbor, N.Y., 2001.

Sarre, Byosistems, 22:311-325, 1989.

Sarver, et al, Science, 247:1222-1225, 1990.

Sattler et al., Science 275:983-986, 1997.

Scanlon et al., Proc. Nat'l Acad. Sci. USA, 88:10591-10595, 1991.

Scheid et al., Proc. Natl. Acad. Sci. USA, 95:7439-7444, 1998.

Schulz et al., Cancer Res., 44:5914-5920, 1984.

Sheldrick et al., Cold Spring Harbor Symp. Quant. Biol., 34:667-678, 1974.

Shen et al., Curr. Opin. Genet. Dev., 5:105-111, 1995.

Shepard et al., J. Virology, 65:787-795, 1991.

Shinoda et al., In: Colloidal Surfactant, Academic Press, 1: 1-96, 1963.

Sieg et al., J. Virology, 8747-8751, 1996.

Sioud et al., J. Mol. Biol., 223:831-835, 1992.

Solodin et al., Biochemistry, 34(41):13537-44, 1995.

Spanjer et al., Biochim. Biophys. Acta, 734(1):40-47, 1983.

Symons, Nucl. Acids Res., 9:6527-6537, 1981.

Symons, Ann. Rev. Biochem., 61:641-671, 1992.

Szoka, et al., Proc. Natl. Acad. Sci. USA, 75:4194-4198, 1978.

Tan et al., J. Biol. Chem., 274:34859-67, 1999.

Tan et al., J. Biol. Chem., 275:25865-25869, 2000.

Temin, In: Gene Transfer, Kucherlapati (ed.), NY, Plenum Press, 149-188, 1986.

Templeton et al., Nat. Biotechnol., 15(7):647-52, 1997.

Thierry et al., Proc. Natl. Acad. Sci. USA, 92(21):9742-6, 1995.

Thompson et al., Nat. Med., 1:277-278, 1995.

Tratschin et al., Mol. Cell. Biol., 4:2072-2081, 1984.

Traunecker et al., EMBO, 10:3655-3659, 1991.

Tsukamoto et al., Nat. Genet., 9:243-248, 1995.

Tur-Kaspa et al., Mol. Cell. Biol., 6:716-718, 1986.

Wadsworth, et al., J. Virology, 15:1487-1497, 1975.

Wagner et al., Science, 260:1510-1513, 1990.

Wang et al., Science, 284:339-343, 1999.

Weinberger et al., Science, 228:740-742, 1985.

White et al., In: The Molecular Basis of Apoptosis in Disease, Tomei and Cope (eds.), Cold Spring Harbor Laboratory Press, 111-141, 1994.

White, Genes and Dev., 7:2277-2284, 1993.

Wilson et al., Science, 244:1344-1346, 1989.

Wolf et al., Comput. Appl. Biosci., 4(1):187-191, 1988.

Wong et al., Gene, 10:87-94, 1980.

Wu et al., Adv. Drug Delivery Rev., 12:159-167, 1993.

Wu et al., Biochemistry, 27:887-892, 1988.

Wu et al., J. Biol. Chem., 262:4429-4432, 1987.

Wyllie et al., Int. Rev. Cytol., 68:251-306, 1980.

Yang et al., Proc. Natl. Acad. Sci. USA, 87:9568-9572, 1990.

Yang et al., Gene Therapy, 4 (9):950-960, 1997.

Yin et al., Nature, 369:321-323, 1994.

Zelenin et al., FEBS Lett., 280:94-96, 1991.

Zha et al., Cell, 87:619-628,1996.

Zhou et al., J. Biol. Chem., 275:25046-25051, 2000.

Zhou et al., J. Virology, 74:11782-11791,2000.

Zhu et al., Chin. J. Biotechno., 9(4):257-261, 1993.

Zhu et al., J. Virology, 69:7960-7970, 1995.

Zufferey et al., Nat. Biotechnol., 15(9):871-875, 1997.

What is claimed is:

1. A method for inducing apoptosis in a cell infected with herpes simplex virus (HSV) comprising administering to the cell a composition comprising an agent that inhibits phosphorylation of BAD by HSV U_{S}3.

2. The method of claim 1, wherein the agent comprises a peptide comprising a sequence of between 4 to 100 contiguous amino acids from SEQ ID NO: 2 or SEQ ID NO: 4, wherein the peptide comprises an amino acid that is a substrate for phosphorylation.

3. The method of claim 2, wherein the amino acid is ser136, ser155, or ser112, or a combination thereof.

4. The method of claim 2, wherein the peptide comprises a sequence of at least 15 contiguous amino acids from SEQ ID NO: 2 or SEQ ID NO: 4.

5. The method of claim 4, wherein the peptide comprises a sequence of at least 20 contiguous amino acids from SEQ ID NO: 2 or SEQ ID NO: 4.

6. The method of claim 5, wherein the peptide comprises a sequence of at least 25 contiguous amino acids from SEQ ID NO: 2 or SEQ ID NO: 4.

7. The method of claim 6, wherein the peptide comprises a sequence of at least 40 contiguous amino acids from SEQ ID NO: 2 or SEQ ID NO: 4.

8. The method of claim 2, wherein the peptide comprises more than one amino acid that is a substrate for phosphorylation.

9. The method of claim 2, wherein the composition further comprises a second peptide comprising a sequence of between 4 to 100 contiguous amino acids from SEQ ID NO: 2 or SEQ ID NO: 4, wherein the second peptide comprises an amino acid that is a substrate for phosphorylation.

10. The method of claim 9, wherein the amino acid that is a substrate for phosphorylation by HSV U_{S}3.

11. The method of claim 1, wherein the agent is a peptide mimetic of a peptide comprising between 4 and 100 contiguous amino acids of SEQ ID NO: 2 or SEQ ID NO: 4, including an amino acid that is a substrate for phosphorylation.

12. The method of claim 1, wherein the agent is a peptide mimetic of a peptide comprising between 4 and 100 contiguous amino acids of SEQ ID NO: 2 or SEQ ID NO: 4, including an amino acid analogue of a phosphorylated amino acid, wherein the amino acid analogue corresponds to one or more of ser112, ser136, or ser152.

13. The method of claim 1, wherein the composition further comprises a lipid.

14. The method of claim 1, wherein the agent is a polypeptide that binds to an amino acid residue of BAD that is a substrate for phosphorylation, wherein the polypeptide inhibits phosphorylation of the amino acid residue, but does not inhibit the pro-apoptotic activity of BAD.

15. The method of claim 14, wherein BAD has an amino acid sequence comprising SEQ ID NO: 2 or SEQ ID NO: 4.

16. The method of claim 14, wherein the amino acid residue that is a substrate for phosphorylation is ser136, ser155, or ser112.

17. The method of claim 14, wherein the polypeptide is an antibody.

18. The method of claim 17, wherein the antibody is a single chain antibody.

19. The method of claim 17, wherein the antibody is a humanized antibody.

20. The method of claim 1, further comprising administering to the cell a BAD polypeptide, wherein the BAD polypeptide comprises at least 100 contiguous amino acids of SEQ ID NO: 2 or SEQ ID NO: 4.

21. The method of claim 20, wherein the polypeptide is administered to the cell by contacting the cell with an expression construct comprising a nucleic acid sequence encoding the BAD polypeptide.

22. The method of claim 21, wherein the expression construct is a viral vector.

23. The method of claim 22, wherein the viral vector is an adenovirus, adeno-associated virus, herpesvirus, lentivirus, or retrovirus.

24. The method of claim 1, further comprising administering to the cell an antiviral agent.

25. The method of claim 24, wherein the antiviral agent is famcyclovir, valacyclovir, or acyclovir.

26. The method of claim 24, wherein the antiviral agent is administered to the patient after the composition is administered.

27. The method of claim 24, wherein the antiviral agent and the composition are administered at the same time to the patient.

28. The method of claim 1, wherein the cell is in a human.

29. A method for treating a patient infected with herpes simplex virus comprising administering to the patient an effective amount of a composition comprising a peptide comprising a sequence comprising between 4 to 100 continuous amino acids of SEQ ID NO: 2 or SEQ ID NO: 4, wherein the peptide comprises ser112, ser135, or ser155, or a combination thereof.

30. A method for treating a patient infected with herpes simplex virus comprising administering to the patient an effective amount of a composition comprising at least two peptides, each peptide comprising a different sequence comprising between 4 to 100 continuous amino acids of SEQ ID NO: 2 or SEQ ID NO: 4, wherein the peptides comprise one, two, or three of ser112, ser135, and ser155.

31. The method of claim 30, wherein the peptides each comprise a sequence of at least 15 contiguous amino acids from SEQ ID NO: 2 or SEQ ID NO: 4.

32. The method of claim 30, wherein the peptides each comprise a sequence of at least 20 contiguous amino acids from SEQ ID NO: 2 or SEQ ID NO: 4.

33. The method of claim 30, wherein the peptides each comprise a sequence of at least 25 contiguous amino acids from SEQ ID NO: 2 or SEQ ID NO: 4.

34. The method of claim 30, wherein the peptides each comprise a sequence of at least 40 contiguous amino acids from SEQ ID NO: 2 or SEQ ID NO: 4.

35. The method of claim 30, wherein at least one peptide comprises more than one amino acid that is a substrate for phosphorylation.

36. The method of claim 35, wherein the amino acid is a substrate for phosphorylation by a HSV U_{S}3.

37. The method of claim 30, further comprising administering to the patient an antiviral agent.

38. The method of claim 37, wherein the antiviral agent is famcyclovir, valacyclovir, or acyclovir.

39. The method of claim 37, wherein the antiviral agent is administered to the patient after the composition is administered.

40. The method of claim 37, wherein the antiviral agent and the composition are administered at the same time to the patient.

41. The method of claim 30, wherein the composition further comprises a lipid.

42. The method of claim 29, further comprising administering to the patient an agent that inhibits the activity of a herpesvirus U_{S}3 protein.

43. A method for blocking BAD-induced apoptosis of a cell comprising providing to a cell suspected of undergoing BAD-induced apoptosis a U_{S}3 polypeptide.

44. The method of claim 43, wherein the U_{S}3 polypeptide is from a herpes simplex virus (HSV) type 1, HSV-2, varicella zoster virus, bovine herpesvirus 1, equine herpesvirus 1, equine herpesvirus 4, galid herpesvirus 1, galid herpesvirus 2, galid herpesvirus 3, cercopithecine herpesvirus 7, cercopithecine herpesvirus 9, simian herpesvirus B, infectious laryngotracheitix virus, canine herpesvirus, or suid virus 1.

45. The method of claim 43, wherein providing comprises introducing into the cell a nucleic acid comprising a nucleic acid sequence encoding a U_{S}3 protein.

46. The method of claim 45, wherein the nucleic acid is an expression construct.

47. The method of claim 46, wherein the nucleic sequence is under the transcriptional control of a promoter active in eukaryotic cells.

48. The method of claim 47, wherein the promoter is a tetracycline controlled promoter.

49. The method of claim 46, wherein said expression vector further comprises a selectable marker.

50. A method of identifying an inhibitor of apoptosis comprising:
a) contacting a BAD peptide or polypeptide with a candidate compound; and
b) assaying the compound for the ability to inhibit the apoptotic activity of the BAD peptide or polypeptide,
wherein the inhibition of the apoptotic activity of the BAD peptide or polypeptide identifies the compound as an inhibitor of apoptotic activity.

51. The method of claim 50, further comprising:
c) assaying the ability of the BAD peptide or polypeptide to promote apoptosis after contacting the BAD peptide or polypeptide with the candidate compound.

52. The method of claim 50, wherein the BAD peptide or polypeptide comprises at 10 contiguous amino acids of SEQ ID NO: 2 or SEQ ID NO: 4.

53. The method of claim 50, wherein the BAD peptide or polypeptide is a BAD polypeptide.

54. The method of claim 53, wherein the BAD polypeptide comprises SEQ ID NO: 2 or SEQ ID NO: 4.

55. The method of claim 51, wherein the BAD peptide or polypeptide is in a cell.

56. The method of claim 50, wherein the BAD peptide or polypeptide comprises ser112, ser135, or ser155.

57. The method of claim 50, wherein a BAD peptide is obtained and contacted with a candidate compound.

58. The method of claim 50, wherein the compound inhibits the activity of the BAD peptide or polypeptide by binding to the BAD peptide or polypeptide.

59. The method of claim 58, wherein the compound inhibits the activity of the BAD peptide or polypeptide by reducing its activity.

60. The method of claim 55, wherein the compound reduces the activity of the BAD peptide or polypeptide activity by reducing the amount of the BAD peptide or polypeptide in the cell.

61. The method of claim 50, wherein the candidate compound is a peptide.

62. The method of claim 50, wherein the candidate compound is a polypeptide.

63. The method of claim 62, wherein the polypeptide is an antibody.

64. The method of claim 50, wherein the candidate compound is a small molecule.

65. The method of claim 50, wherein the candidate compound is a peptide mimetic.

66. The method of claim 50, further comprising:
c) comparing the BAD peptide or polypeptide activity after being contacted with the candidate compound to a second BAD peptide or polypeptide not contacted with the candidate compound.

67. The method of claim 51, further comprising:
d) incubating the BAD peptide or polypeptide with a U_{S}3 polypeptide prior to step c).

68. The method of claim 50, wherein the BAD peptide or polypeptide is in a cell.

69. The method of claim 68, wherein assaying for inhibition of activity of the BAD peptide or polypeptide comprises assaying for an amount of the BAD peptide or polypeptide.

70. The method of claim 50, wherein assaying for inhibition of activity of the BAD peptide or polypeptide comprises assaying the BAD peptide or polypeptide for phosphorylation.

71. The method of claim 70, wherein the BAD peptide or polypeptide is assayed for phosphorylation at ser112, ser135, or ser155.

72. A method of screening for a promoter of apoptosis comprising:
a) incubating a BAD peptide or polypeptide with a U_{S}3 polypeptide and a candidate compound;
b) assaying the compound for an ability to relieve inhibition of apoptosis by the U_{S}3 polypeptide on the BAD peptide or polypeptide,
wherein the relief of inhibition of apoptosis by the compound identifies it as a promoter of apoptosis.

73. The method of claim 72, further comprising:
c) comparing the inhibition of apoptosis by U_{S}3 on the BAD peptide or polypeptide in the absence of the candidate compound.

74. The method of claim 72, wherein the U_{S}3 polypeptide comprises SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, or SEQ ID NO: 20.

75. An apoptosis modulator identified by a process comprising:
a) contacting a BAD peptide or polypeptide with a candidate compound;
b) assaying the compound for an ability to modulate the BAD peptide or polypeptide, wherein the compound modulates the BAD peptide or polypeptide.

76. The apoptosis modulator of claim 75, wherein the process further comprises:
c) incubating the BAD peptide or polypeptide with a U_{S}3 polypeptide; and
d) comparing the BAD peptide or polypeptide after being contacted with the candidate compound to a second BAD peptide or polypeptide not contacted with the candidate compound.

77. The apoptosis modulator of claim 75, wherein the apoptosis modulator inhibits apoptosis.

78. A method for blocking apoptosis of a cell comprising providing to the cell a herpesvirus U_{S}3 polypeptide.

79. The method of claim 78, wherein the U_{S}3 polypeptide is from a herpes simplex virus (HSV) type 1, HSV-2, varicella zoster virus, bovine herpesvirus 1, equine herpesvirus 1, equine herpesvirus 4, galid herpesvirus 1, galid herpesvirus 2, galid herpesvirus 3, cercopithecine herpesvirus 7, cercopithecine herpesvirus 9, simian herpesvirus B, infectious laryngotracheitix virus, canine herpesvirus, or suid virus 1.

80. The method of claim 78, wherein providing comprising introducing into the cell nucleic acid comprising a nucleic acid sequence encoding the U_{S}3 polypeptide.

81. The method of claim 80, wherein the nucleic acid is an expression vector.

82. The method of claim 81, wherein the expression vector is a viral vector.

83. The method of claim 76, wherein the U_{S}3 polypeptide comprises an amino acid sequence of SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, or SEQ ID NO: 20.


SEQUENCE LISTING (25 sequences)

SEQ ID NO: 1 - DNA, 945 bp, Human BAD
  CDS (52)..(555)
gggcctagggcgccgggtcaggggcctcgagatcgggcttgggcccagagcatgttccagatcccagagtttgagccgagtgagcaggaagactccagctctgcagagaggggcctgggccccagccccgcaggggacgggccctcaggctccggcaagcatcatcgccaggccccaggcctcctgtgggacgccagtcaccagcaggagcagccaaccagcagcagccatcatggaggcgctggggctgtggagatccggagtcgccacagctcctaccccgcggggacggaggacgacgaagggatgggggaggagcccagcccctttcggggccgctcgcgctcggcgccccccaacctctgggcagcacagcgctatggccgcgagctccggaggatgagtgacgagtttgtggactcctttaagaagggacttcctcgcccgaagagcgcgggcacagcaacgcagatgcggcaaagctccagctggacgcgagtcttccagtcctggtgggatcggaacttgggcaggggaagctccgccccctcccagtgaccttcggtccacatcccgaaatccacccgttcccattgccctgggcagccattttgaatatgggaggaagtaagttccctcaggcctatgcaaaaagaggatccgtgctgtatcctttggagggagggttgacccagattcccttccggtgtgtgtgaagccacggaaggttggtcccatcggaagttttgggttttccgcccacagccgccggaagtggctccgtggccccgccctcaggttccggggtttcccccaggcgcctgcgctaagtagcgagccaggtttaaccgttgtgtcaccgggacccgagcccccgcgatgccctgggggccgtgatcagtaccaaatgttaataaagcccgcgtgtgtgccaaaaaaaaaaaa

SEQ ID NO: 2 - protein, 168 aa, Human BAD
MFQIPEFEPSEQEDSSSAERGLGPSPAGDGPSGSGKHHRQAPGLLWDASHQQEQPTSSSHHGGAGAVEIRSRHSSYPAGTEDDEGMGEEPSPFRGRSRSAPPNLWAAQRYGRELRRMSDEFVDSFKKGLPRPKSAGTATQMRQSSSWTRVFQSWWDRNLGRGSSAPSQ

SEQ ID NO: 3 - DNA, 1454 bp, Mus musculus
ggcacgagcggaccccgccccctagcttgtgtctgcaggccccgcgtccggcccggggcagcgtacgcacacctatcctggcagcagaggcccctggagccccacggctcgccttcctgggcgccccgtcccttcttccgcacccgggcggccatcctgccgtaaaggagctcccgaaatggcgcggggggttgtccccaagacgggcagtgcaaggccctccacgatcgggaagaaggagctggtcttcccatcccggtcactcggtccagggggagcaataaccatcgcaacgaccattgcatccgacggccgagcttcagtgaacggctctataagtaatcactaagctgtttacagagttttcaccagctccccagggaggtgtcattaaccccattttacaggagggaattcgggcccagaagggctggaggacttatcagccgaagcaggcctccaggatccaaatgggaaccccaaagcagccctcgctggctcctgcacacgccctaggcttgaggaagtccgatcccggaatccggagcctggggagcgacgcgggaggaaggcggtggagaccagcagcccagagtatgttccagatcccagagtttgagccgagtgagcaggaagacgctagtgctacagataggggcctgggccctagcctcactgaggaccagccaggtccctacctggccccaggtctcctggggagcaacattcatcagcagggacgggcagccaccaacagtcatcatggaggcgcaggggctatggagactcggagtcgccacagttcgtacccagcggggaccgaggaggatgaagggatggaggaggagcttagcccttttcgaggacgctcgcgttcggctccccccaatctctgggcagcgcagcgctacggccgtgagctccgaaggatgagcgatgagtttgagggttccttcaagggacttcctcgcccaaagagcgcaggcactgcaacacagatgcgacaaagcgccggctggacgcgcattatccagtcctggtgggatcgaaacttgggcaaaggaggctccaccccctcccagtgatcttctgctccacatcccggaactctacccgctcccgtcgcccgccatattgggtgtgggcggaagtctttcgaggccttaggaaaaaaaaagaggatcgctgtgtccctttaacagggagaagagctgacgtacagcttgagtcccttccggtgcgtgcaatagccacggaggggtggctcctgtttggagtttcaaagttttccacgcaccccaccccctaagcctccggaagtggctgttttccctctcctgttctggactgccctcgggtgcctgtgctaagttgggggtctgggtgctgtcctgtcataactggggacccgaggtcgcgagaaacgtgctttataataaagcctgcgcatgtgc

SEQ ID NO: 4 - protein, 204 aa, Mus musculus
MGTPKQPSLAPAHALGLRKSDPGIRSLGSDAGGRRWRPAAQSMFQIPEFEPSEQEDASATDRGLGPSLTEDQPGPYLAPGLLGSNIHQQGRAATNSHHGGAGAMETRSRHSSYPAGTEEDEGMEEELSPFRGRSRSAPPNLWAAQRYGRELRRMSDEFEGSFKGLPRPKSAGTATQMRQSAGWTRIIQSWWDRNLGKGGSTPSQ

SEQ ID NO: 5 - DNA, 605 bp, Herpes Simplex Virus
gatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatcctgcatataagcagctgctttttgcctgtactgggtctctctggttagaccagatttgagcctgggagctctctggctaactagggaacccactgcttaagcctcaataaagcttgccttgagtgcttcaacgatcgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgc

SEQ ID NO: 6 - protein, 481 aa, Herpes Simplex Virus
MACRKFCRVYGGQGRRKEEAVPPETKPSRVFPHGPFYTPAEDACLDSPPPETPKPSHTTPPSEAERLCHLQEILAQMYGNQDYPIEDDPSADAADDVDEDAPDDVAYPEEYAEELFLPGDATGPLIGANDHIPPPCGASPPGIRRRSRDEIGATGFTAEELDAMDREAARAISRGGKPPSTMAKLVTGMGFTIHGALTPGSEGCVFDSSHPDYPQRVIVKAGWYTSTSHEARLLRRLDHPAILPLLDLHVVSGVTCLVLPKYQADLYTYLSRRLNPLGRPQIAAVSRQLLSAVDYIHRQGIIHRDIKTENIFINTPEDICLGDFGAACFVQGSRSSPFPYGIAGTIDTNAPEVLAGDPYTTTVDIWSAGLVIFETAVHNASLFSAPRGPKRGPCDSQITRIIRQAQVHVDEFSPHPESRLTSRYRSRAAGNNRPPYTRPAWTRYYKMDIDVEYLVCKALTFDGALRPSAAELLCLPLFQQK

SEQ ID NO: 7 - protein, 481 aa, Herpes Simplex Virus
MACRKFCGVYRRPDKRQEASVPPETNTAPAFPASTFYTPAEDAYLAPGPPETIHPSRPPSPGEAARLCQLQEILAQMHSDEDYPIVDAAGAEEEDEADDDAPDDVAYPEDYAEGRFLSMVSAAPLPGASGHPPVPGRAAPPDVRTCDTGKVGATGFTPEELDTMDREALRAISRGCKPPSTLAKLVTGLGFAIHGALIPGSEGCVFDSSHPNYPHRVIVKAGWYASTSHEARLLRRLNHPAILPLLDLHVVSGVTCLVLPKYHCDLYTYLSKRPSPLGHLQITAVSRQLLSAIDYVHCKGIIHRDIKTENIFINTPENICLGDFGAACFVRGCRSSPFHYGIAGTIDTNAPEVLAGDPYTQVIDIWSAGLVIFETAVHTASLFSAPRDPERRPCDNQIARIIRQAQVHVDEFPTHAESRLTAHYRSRAAGNNRPAWTRPAWTRYYKIHTDVEYLICKALTFDAALRPSAAELLRLPLFHPK

SEQ ID NO: 8 - protein, 393 aa, Herpes Simplex Virus
MNDVDATDTFVGQGKFRGAISTSPSHIMQTCGFIQQMFPVEMSPGIESEDDPNYDVNMDIQSFNIFDGVHETEAEASVALCAEARVGINKAGFVILKTFTPGAEGFAFACMDSKTCEHVVIKAGQRQGTATEATVLRALTHPSVVQLKGTFTYNKMTCLILPRYRTDLYCYLAAKRNLPICDILAIQRSVLRALQYLHNNSIIHRDIKSENIFINHPGDVCVGDFGAACFPVDINANRYYGWAGTIATNSPELLARDPYGPAVDIWSAGIVLFEMATGQNSLFERDGLDGNCDSERQIKLIIRRSGTHPNEFPINPTSNLRRQYIGLAKRSSRKPGSRPLWTNLYELPIDLEYLICKMLSFDARHRPSAEVLLNHSVFQTLPDPYPNPMEVGD

SEQ ID NO: 9 - protein, 468 aa, Bovine herpesvirus 1
MERAAERLARQRARGLWRSRFACCVAAEPSGEPARPERSRSRCGSARCAAAGSADLYLAVNNEGPEVAPPARTGPPDADGIEGGAAVGNEQGGVAAGNERRAAIGDKKKSASGGENESESESESESESGSESGADDGDWDDDDDAGPAGGVTREEAEGAARALNFRIIRRLTPGSEGRVFEATGPAPAQEHVVLKIGASASTLAEAMLLRTLDHANVVKLKAVLFHGELVCVVLARYREDLHTHLWRINRPLALPAALAVTRAVLRGLAYLHSRRIAHRDVKTENVFLNGPGDVCLGDFGAAHGPVTEPRYYGLAGTLETNSPELLARARYDCRTDVWSAGVVAYEMLAYPRALFDSPAGPQGEDAEASGPPTILGDRDCARQLLRVIRRLAVHAEEFPPSPTDRLTRNFKRHASTRREPHSPYRCLAVLRLPCDADRLLHQMLTFDFRARPTAAELLEHPVFGAASG

SEQ ID NO: 10 - protein, 382 aa, Equine herpesvirus 1
MENKQCDHLTDWFSTTSDASESMDTTPPLPPPTPSVDPSYSGAAADEDLYSDISEGDLEYSDCDSASESDEDDDDCLIPSKEKAREVAASFGYTVIKTLTPGSEGRVMVATKDGQPEPVVLKIGQKGTTLIEAMMLRNVNHPSVIQMKDTLVSGAITCMVLPHYSSDLYTFLTKESRRIPIDQALIIEKQILEGLRYLHAQRIIHRDVKTENIFINSVDQVCIADFGAAQFPVVEPADLGLAGTVETNAPEVLARAKYNSKADIWSAGIVLFEMLAYPSTLFEDPPSTPEEYVKSCHSQLLKIISTLKINPEEFPRDPGSRLVRGYIEYSRLERKPYTRYPCFQRVNLHIDGEFLVHKMLAFNAAMRPSAEELLSYPMFAQL

SEQ ID NO: 11 - protein, 384 aa, Equine herpesvirus 4
MENKQYDHLLSDWLSGNISEASESMDTTPPLQLSVHPQNPSCGGAAANEDLYSDISDGDLECSDCDSASESDEDDDDGLMPPKEKAKEVAASFGFKVIKTLTPGSEGRVMVATKEGQPDQVVLKIGQKGTTLIEAMMLRNVNHPCVIKMKDTLVSGGITCMVLPHYNSDLYTFLTRRSTRIPIDQALIIERQILEGLRYLHAQRIIHRDVKTENIFINSVDQVCIADFGAAQFPVVDPMDLGLAGTVETNAPEVLARAKYNSKVDIWSAGIVLFEMLAYPSTLFEDPPSTPQEYVKSCHSQLLRIISKLKINPEEFPREPESRLVRGYIEYASLERKPHTRYPCFQRVNLHIDGEFLIHKMLAFNAAMRPSAEELLSYPMFMNL

SEQ ID NO: 12 - protein, 402 aa, Gallid herpesvirus 1
MSSSPEAETMECGISSSKVHDSKTNTTYGIIHNSINGTDTTLFDTFPDSTDNAEVTGDVDDVKTESSPESQSEDLSPFGNDGNESPETVTDIDAVSAVRMQYNIVSSLSPGSEGYIYVCTKRGDNTKRKVIVKAVTGDKTLGSEIDILKKMSHRSIIRLVHAYRWKSTVCMVMPKYKCDLFTYIDIMGPLPLNQIITIERGLLGALAYIHEKGIIHRDVKTENIFLDKPENVVLGDFGAACKLDEHTDKPKCYGWSGTLETNSPELLALDPYCTKTDIWSAGLVLFEMSVKNITFFGKQVNGSGSQLRSIIRCLQVHPLEFPQNNSTNLCKHFKQYAIQLRHPYAIPQIIRKSGMTMDLEYAIAKMLTFDQEFRPSAQDILMLPLFTKEPADALYTITAAHM

SEQ ID NO: 13 - protein, 402 aa, Gallid herpesvirus 2
MSSSPEAETMECGISSSKVHDSKTNTTYGIIHNSINGTDTTLFDTFPDSTDNAEVTGDVDDVKTESSPESQSEDLSPFGNDGNESPETVTDIDAVSAVRMQYNIVSSLSPGSEGYIYVCTKRGDNTKRKVIVKAVTGGKTLGSEIDILKKMSHRSIIRLVHAYRWKSTVCMVMPKYKCDLFTYIDIMGPLPLNQIITIERGLLGALAYIHEKGIIHRDVKTENIFLDKPENVVLGDFGAACKLDEHTDKPKCYGWSGTLETNSPELLALDPYCTKTDIWSAGLVLFEMSVKNITFFGKQVNGSGSQLRSIIRCLQVHPLEFPQNNSTNLCKHFKQYAIQLRHPYAIPQIIRKSGMTMDLEYAIAKMLTFDQEFRPSAQDILMLPLFTKEPADALYTITAAHM

SEQ ID NO: 14 - protein, 391 aa, Gallid herpesvirus 3
METNELSSKVSDYNANRPYETIRSDTSDTDPSVSCGTLSDKDGDDEESIDLSKVPNATNVGAGEDCTSPNDGRTELCRTTSVTGPASVVRMQYNIISPLPPSSEGRVFVCTRWDDVSNKKVIVKVVTGGRDPGREIEIVKTLSHCAIIQLIHAYSWKSTVCMVMRKYKCDLFTYVDRKESIPLKDVIVIERRLLEALVYLHGKGVIHRDVKTENIFLDYPGNAVLGDFGAACKLDMHDNSPKCYGWAGTMETNSPELLALDPYCAKTDIWSAGLVLFEMSAKKRTLFGKQVKTSSSQLRALIRCLQIHALEFPQDESTTLCKQFKQYAIPLRPPFSIPEVVRRNIPSMDVEYTIAKMLTFDQEFRPSAQDILAFPLFVKEAPQNLQALFVP

SEQ ID NO: 15 - protein, 345 aa, Cercopithecine herpesvirus 7
MNYDDDCLYEDKHMDTDIYDMLADEDTSDVDNTLAVCATARAGIEKAGFSVLETFTPGAEGFTFACIENKTRENVVIKAGQRGGTVTEAHILRNINHPVIIRLMGTFTYNSFTCLVLPRYKTDLYCYLSDRRRIAICDMLSIERSVLRAIQYLHENRIIHRDVKAENIFINHPGDVCLGDFGAACYPVDITQNKYYGWAGTIATNAPELLARDPYGPAVDIWSAGIVLFEMATCHDSLFEKDGLDGDCDSDRQIKLIIRRTGVHPSEFPIDAQATLDEIYRTCQKTSRKPGTRPTWTNLYELPLELEYLICKMLAFDAHKRPSAKALLDFAAFYDIPDPYPNPTN

SEQ ID NO: 16 - protein, 345 aa, Cercopithecine herpesvirus 9; identical to SEQ ID NO: 15

SEQ ID NO: 17 - protein, 147 aa, Simian Herpes B
VDPAHYGIAGTVDTNAPEVLAGDPYTPSVDIWSAGLVIFEAAVHTASLFSVSRTDEQRPYDAQILRIIQQAQVHVDEFPQRAGSRLVSQYRHRAARNRRPPHTRPAWTRYYKLDLDVEYLVCRALTFDGARRPSAAELLRLPLFQSS

SEQ ID NO: 18 - protein, 402 aa, Infectious Laryngotracheitis Virus
MSSTPEAETMECGISSSKVHDSKTNTTYGIIHNSINGTDTTLFDTFPDSTDNAEVTGDVDDVKTESSPESQSEDLSPFGNDGNESPETVTDIDAVSAVRMQYNIVSSLPPGSEGYIYVCTKRGDNTKRKVIVKAVTGGKTLGSEIDILKKMSHRSIIRLVHAYRWKSTVCMVMPKYKCDLFTYIDIMGPLPLNQIITIERGLLGALAYIHEKGIIHRDVKTENIFLDKPENVVLGDFGAACKLDEHTDKPKCYGWSGTLETNSPELLALDPYCTKTDIWSAGLVLFEMSVKNITFFGKQVNGSGSQLRSIIRCLQVHPLEFPQNNSTNLCKHFKQYAIQLRHPYAIPQIIRKSGMTMDLEYAIAKMLTFDQEFRPSAQDILMLPLFTKEPADALYTITAAHM

SEQ ID NO: 19 - protein, 340 aa, Canine Herpes Simplex Virus
MAKCTTEKFCCISVNRESSVDPEDFYKPVPLTSDLIEEDNLHQDKIMDEDLYSDFSDDDFMDYTKNPTESENERESDEEVEESYESDEDKKSLSPTKSEGIEAAEALKFSVVKSLTPGSEGRVFIALKKDKDTSYKVILKIGQRGNTLVESLILRNISHQSIIKLQDTLFYKELTCLVLPYYKYDLYNFLMDHGKSLSFESVIKIEKQILTGLQYIHGKKIIHRDIKTENIFLDNDSNVCIGDFGASQFPVSSPDYLGIAGTIETNAPEVLSKDAYNCKADIWSAGIILFEMLAYPNVLFEEEERDSSDLINNCNLHLIKIISTLKINPNEFPSDLESNL

SEQ ID NO: 20 - protein, 334 aa, SUID HSV 20 (Straind KA)
MADAGIPDEILYSDISDDEIIIDGDGDSSGDEDDDDGGLTRQAAARIVTDLGFEVLQPLQSGSEGRVFVARRPGEADTVVLKVGQKPSTLMEGMLLQRLSHDNVMRMKQMLARGPATCLVLPHFRCDLYSYLTMRDGPLDMRDAGCVIRAVLRGLAYLHGMRIMHRDVKAENIFLEDVDTVCLGDLGAARCNVAAPNFYGLAGTIETNAPEVLARDRYDTKVDVWGAGVVLFETLAYPKTITGGDEPAINGEMHLIDLIRALGVHPEEFPPDTRLRSEFVRYAGTHRQPYTQYARVARLGLPETGAFLIYKMLTFDPVRRPSADEILNFGMWTV

SEQ ID NO: 21 - protein, 10 aa, Artificial Sequence
  Description of Artificial Sequence Herpes Simplex Virus
RRRRTRRSRE

SEQ ID NO: 22 - protein, 6 aa, Herpes Virus
RRRRSR

SEQ ID NO: 23 - protein, 6 aa, Artificial Sequence
  Description of Artificial Sequence Kinase substrate
RRRRTR

SEQ ID NO: 24 - protein, 6 aa, Artificial Sequence
  MOD_RES (4): Xaa = Anything
RRRXTY

SEQ ID NO: 25 - protein, 6 aa, Artificial Sequence
  MOD_RES (4): Xaa = anything
RRRXSY